(19) United States
(12) Patent Application Publication
(10) Pub. No.: US 2026/0053724 A1
(43) Pub. Date: Feb. 26, 2026

(54) ORAL CARE COMPOSITIONS

(71) Applicant: Soluta Consult GmbH, Düsseldorf (DE)

(72) Inventor: Dana ADYANI-FARD, Meerbusch (DE)

(21) Appl. No.: 19/104,827

(22) PCT Filed: Aug. 24, 2023
(86) PCT No.: PCT/EP2023/073288

§ 371 Date: Feb. 19, 2025

(30) Foreign Application Priority Data
Aug. 26, 2022 (DE) 10 2022 003 105.1

(51) Int. Cl. A61K 8/73; A61K 8/31; A61K 8/34; A61K 8/35; A61K 8/41; A61K 8/43; A61K 8/44; A61K 8/49; A61K 8/81; A61K 8/9789; A61Q 11/00

(52) U.S. Cl. CPC A61K 8/735; A61K 8/31; A61K 8/342; A61K 8/345; A61K 8/35; A61K 8/41; A61K 8/43; A61K 8/44; A61K 8/4926; A61K 8/4973; A61K 8/498; A61K 8/731; A61K 8/8147; A61K 8/9789; A61Q 11/00

(57) ABSTRACT

The invention refers to novel oral care compositions and their uses.

### BACKGROUND

The oral epithelial barrier (including the gingival epithelial barrier) separates the host from the environment and provides the first line of defense against pathogens, exogenous substances, and mechanical stress. Disruption of the gingival epithelial barrier and subsequent invasion by exogenous pathogens into the host tissue triggers an inflammatory response and establishes chronic infection.

The aligner splint therapy is a therapy concept for the orthodontic harmonization of the dental arch by wearing removable dental splints for several hours. Such aligner splints are individually adapted to the dentition of a user and cover both the teeth and part of the gingival of a user. Wearing the aligner splint for a long time poses a risk of irritation of the gums, gingival recession as well as an excessive proliferation of bacteria between the aligner splint and the covered tooth and gingival surface. Aligner splint users often complain of bad breath and a dry mouth in the morning after wearing an aligner overnight.

Cosmetic oral hygiene products such as toothpastes, mouthwashes or mouthwashes are usually “rinse-off products”, i.e., they only work for a short period of time and are therefore not suitable for the problems occurring while wearing aligner splints.

EP 3 220 875 describes a toothpaste with high levels of natural calcium carbonate, sodium chloride and an antimicrobial effective amount of a zinc salt. However, this care composition is not suitable for eliminating the problems of wearing aligner splints. EP 1 888 014 describes a dental care composition for improving the mineralization of teeth. This category includes the commercially available MI Paste Plus. WO 2021 168684 describes an oral care composition free of jasmonic acid, gibberellic acid and/or zeatin compounds. These oral care compositions are dissimilar to the oral care compositions of the present invention and are not suitable for alleviating the problems of aligner wearers. US 2007/0003502 is focused on the use of compositions with at least 5% maltose or trehalose. Although hair tonic, bath salt and cosmetic creams as well as mouse wash and toothpaste (Examples 48 to 50) are disclosed, the examples either contain no hyaluronic acid, at all, or only 0.1% hyaluronic acid. US 2020/0390676 is focused on oral care compositions with hyaluronic acid having specific viscosities based on the amount of hyaluronic acid and optionally a further polymer. Although the document discloses various oral care compositions, none of them has a content of xylitol of more than 2%. Moreover, there is no teaching to use a further anti-inflammatory substance or catechin. KR1020000031162 refers to oral compositions with triclosan and hyaluronic acid, however, the application does neither disclose nor suggest a composition according to the invention. Furthermore, triclosan can result in contact dermatitis, or skin irritation and an increase in allergic reactions and is a potential endocrine disruptor, for example, triclosan will be banned in mouthwash in 2023 in the European Union (see coslaw.eu). Moreover, the examples in KR1020000031162 are not suitable as compositions according to the invention, i.e., the application does not provide the skilled person with the knowledge to arrive at the compositions according to the present invention.

There is a need for oral care compositions that can counteract these dangers and result in improved mouthfeel. The present invention refers to oral care compositions which, compared to conventional oral care compositions, have improved care potential due to the specific combination of different care components and a longer exposure time (inhibition of biofilm formation (usually bacteria and their metabolization products), preventing and cure of inflammatory and support of regeneration of damaged tissue). It is even possible to obtain the individual components easily from renewable (biological) raw materials. The care or preventative substances in such an oral care composition as well as the care composition should preferably be easy to handle and can be used outside dental offices. Through the oral application of an oral care composition according to the invention, care and supportive main tasks are achieved.

### Definitions

The term “a” as used herein generally refers to the indefinite article and means “one or more than one” e.g., 1, 2, 3 etc. If the term “a” refers to only a single item, this is either clear from the context or explicitly stated in the form of “a (1)”.

The term “abrasive” refers to solid particles which are usually present in an oral care composition such as toothpastes. Well-known abrasives for oral compositions are calcium-containing, silica-, carbonate-, phosphate-, alumina-abrasives, and other suitable abrasives. Non limiting examples are calcium carbonate, dicalcium phosphate, tricalcium phosphate, calcium orthophosphate, calcium metaphosphate, calcium polyphosphate, calcium hydroxyapatite, calcium carbonate, strontium carbonate, calcium phosphate, sodium hexametaphosphate, dicalcium phosphate, tricalcium phosphate, calcium orthophosphates, calcium metaphosphates, fused silica, fumed silica, precipitated silica, hydrated silica, diatomaceous earth, barium sulfate, wollastonite, perlite, alumina (Al₂O₃), polymethylmethacrylate particles, tospearl, and combinations of any of the foregoing. Abrasives such as silica abrasives may have an average particle size ranging from 0.05 to 30 μm. Abrasives are commercially available, e.g., “Syloid”, or Zeodent®, particularly the silicas carrying the designation Zeodent® 119, Zeodent® 118, Zeodent® 109 and Zeodent® 129.

“Additional components” are any compounds known at the filing date of the present application, which can be used in oral care compositions. The most interesting ones are listed in group (e) and can be categorized or described according to their cosmetic and/or therapeutic benefits or their postulated mode of action or function. It is to be understood, however, that the further compounds may, in some instances, provide more than one cosmetic and/or therapeutic benefit or effect through more than one mechanism of action or function. Therefore, the classifications in (e) herein are made for convenience and are not intended to limit any ingredient to the functions or activities specifically listed. The term “further components” also includes those compounds that occur in extracts and oils in addition to components from groups (a) to (d). A precise characterization of these components is not necessary, but the total amount of these components is required. This can be calculated by simply subtracting the amount of components from groups (a) to (d) from the amount of extract or oil used. In other words, compounds of group (a) to (d) or (e) may have also other beneficial effects other than those of their respective class. For example, glycerol or ethylene glycol are primary polar solvents but also have a humectant effect. Nevertheless, they will not be mentioned as humectants in group (e) but are already counted in group (a) for estimating the amount of the various compounds in the present compositions. Catechins from tea can have an antioxidative effect as well as a vasodilatation effect but will of course only counted once when determining the amount of further components (e) in a formulation.

“Allantoin” (5-ureidohydantoin) is an oxidation product of uric acid. Allantoin helps with wounds and skin irritations and stimulates the growth of healthy tissue.

“Carbomer” as used herein refers to synthetically produced polyacrylic acids (PAAs), typically cross-linked with polyalkene ethers of sugars or polyalcohols. These can be homopolymers of acrylic acid cross-linked with an allyl ether of pentaerythritol, allyl ether of sucrose or allyl ether of propylene. Polyacrylic acid occurs in different degrees of polymerization. Depending on the degree of polymerization (molecular weight), the carbomer gels differ in their viscosity and thus also in their cosmetic, pharmaceutical, and technical application. In an aqueous solution at neutral pH, PAA is an anionic polymer, i.e. many of the side chains of PA lose their protons and are negatively charged. This gives PAA-polyelectrolytes the ability to absorb and hold water, allowing them to swell to many times their original volume. High molecular weight and cross-linked polyacrylic acid is also known under the trade name Carbomer. Well-known trade names are Carbopol®, Pemulen® and Noveon®. They are mostly polymers of acrylic acid cross-linked with polyalkenyl ethers or divinyl glycol. The numbers behind the name define a mixture of molar volume and viscosity of a 2% solution at 20° C. in mPa*s. According to the USP (US Pharmacopoeia), three types of carbomers are generally classified, which can be distinguished by pharmaceutically relevant viscosities:
Type A (4 to 11 Pa*s) (e.g., Carbopol 981, Carbopol 971 or Carbopol 71G),
Type B (25-45 Pa*s (e.g., Carbopol 974P, Carbopol 984, Carbopol 5984), and
Type C (40 to 60 Pa*s) (e.g., Carbopol 980),
   Commonly used carbomers are Carbomer 50,000 (used in e.g., Carbopol 940), and Carbomer 35000 (used in e.g. Carbopol 974P and Carbomergel pH 6.5 NRF p. 43)). The presence of cations (e.g., Ca²⁺, Al³⁺) can lead to liquefaction or coagulation (flocculation) of the gel. In such cases, the complexing of the polyvalent cations with, for example, sodium edetate (Na₂EDTA) has a stabilizing effect.

“Catechins” as used herein are polyphenolic plant metabolites from the group of flavonoids and are therefore generally classified as secondary plant substances. They are derived from flavan-3-ol.

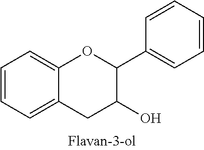 [SMILES: OC1Cc2ccccc2OC1c1ccccc1]

The four most important catechins are catechin, epicatechin (each with molecular formula C₁₅H₁₄O₆), gallocatechin and epigallocatechin (each with molecular formula C₁₅H₁₄O₇).

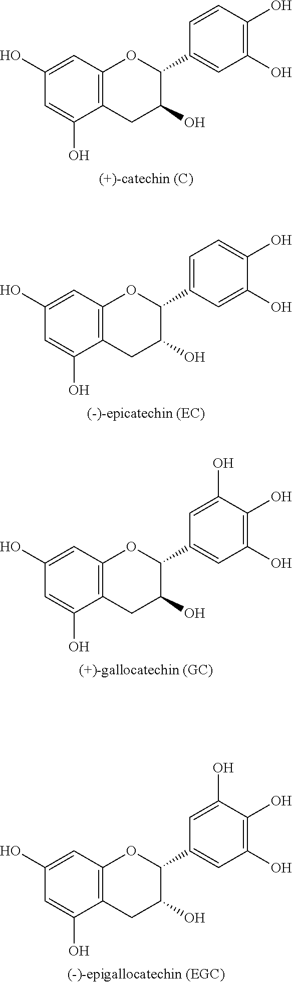 [SMILES: Oc1cc(O)c2c(c1)O[C@H](c1ccc(O)c(O)c1)[C@@H](O)C2 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@@H](O)C2 ; Oc1cc(O)c2c(c1)O[C@H](c1cc(O)c(O)c(O)c1)[C@H](O)C2]

Each of these substances occurs in two isomers. (+)-catechin (C) (CAS 154-23-4) e.g., has a 2R,3S configuration while (−)-catechin (CAS 18829-70-4) has a 2S,3R configuration.

The term catechin also encompasses esters (e.g., with gallic acid or 4-fatty acid ethers of gallic acid, such as 4-palmitoyl-gallic acid) of C, EC, GC, and EGC. E.g., epigallocatechin gallate (EGCG)—epigallocatechin-3-gallate) is the ester of epigallocatechin and gallic acid.

Catechins originating from green tea are, e.g., EC, EC gallate (ECG), EGC, and EGC gallate (EGCG). The amounts may slightly vary depending on the tea variety but the total amount of said four catechins in green tea leaves is generally between 30% and 40% based on the dry weight of green tea leaves. The ratio between the four catechins EC, ECG, EGC and EGCG in green tea is around 5:10:15:30.

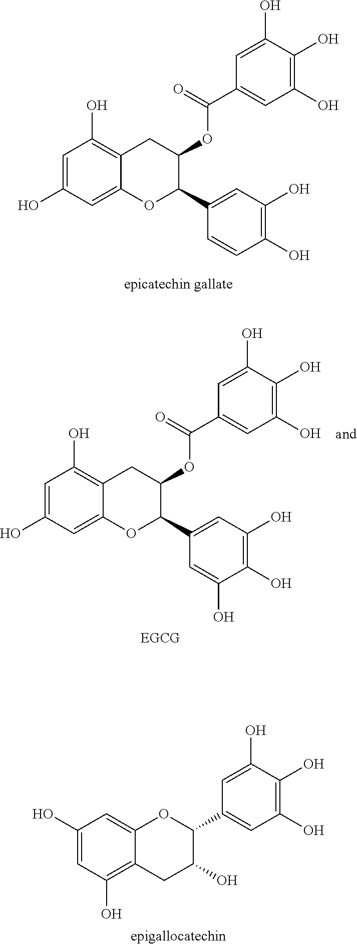 [SMILES: O=C(O[C@@H]1Cc2c(O)cc(O)cc2O[C@@H]1c1ccc(O)c(O)c1)c1cc(O)c(O)c(O)c1 ; O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1cc(O)c(O)c(O)c1)[C@H](O)C2]

As used herein, the term “compatible” means that the components of the composition can be mixed without interfering in a manner that would significantly reduce the stability and/or effectiveness of the composition.

The term “flavonoids” describes polyphenolic secondary metabolites found in plants. They have antioxidant properties and are important for nutrition. They are generally derived from 2-phenyl-1,4-benzopyrone. The term “flavonoids” also encompasses isoflavonoids (derived from 3-phenyl-1,4-benzopyrone) and neoflavonoids, derived from 4-phenyl-1,2-benzopyrone. The three flavonoid classes above are all ketone-containing compounds and as such, anthoxanthins (flavones and flavonols). The skilled person is aware which compounds fall under the term flavonoids at the filing date of this application.

“Gibberellic acid” includes a class of compounds also known as gibberellins. Gibberellins are plant hormones and tetracyclic diterpenoid acids.

The term “gum care” refers to benefits aimed at relieving one or more symptoms of the earlier stage of gum disease (i.e., gingivitis), which includes: relief of red, swollen, tender gum and/or gingivitis.

The term “gum health” as used herein refers to inherently promoted benefits of an oral care composition to provide “gum health” benefits which include at least improvement in gingival and periodontal wound healing, as well as improved resilience and strengthening of bacterial resorption, including early damage to periodontitis.

“Humectants” serve to keep an oral care composition from hardening upon exposure to air, to provide a moist mouthfeel and, with certain humectants, to impart a desirable sweet taste. Suitable humectants for the present invention include edible polyhydric alcohols such as glycerin, sorbitol, erythritol, butylene glycol, polyethylene glycol, and combinations thereof.

“Hyaluronic acid” (HA) as used herein is a glycosaminoglycan. The term “HA” as used herein also includes the salt of HA, such as the sodium (Na), lithium (Li) or potassium (K) salt of HA, and the magnesium (Mg) or calcium (Ca) salt of HA. The term “hyaluronic acid” as used herein also includes thiolated HAs (see e.g., Kafedjiiski et al., Int. J. Pharm., 343, (2007) 48-58), such as HA-cysteine ethyl ester conjugates, and their salts, e.g., their alkali and alkaline earth metal salts. HA occurs as a macromolecular chain of disaccharides composed of D-glucuronic acid and N-acetyl-D-glucosamine. The disaccharides are linked by a β(1→4) glycosidic bond. The term HA as used herein encompasses chains typically composed of about 250 (about 95 kDa) to about 50,000 (about 18,966 kDa) disaccharide repeat units (n=about 250 to about 50,000)

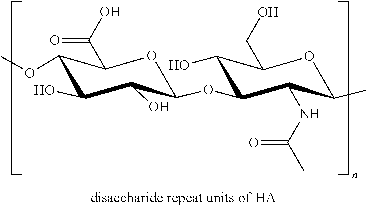

as well as degradation fragments of typically occurring hyaluron chains up to a D-glucuronic acid N-acetyl-D-glucosamine disaccharide in which the glucuronic acid is glycosidically β(1→3) linked to the N-acetyl-D-glucosamine. Such degradation fragments usually have a molecular weight of 5 kDa to 150 kDa (degradation fragments with a molecular weight of 5 kDa to 130 kDa are preferably used in cosmetics). Also, the term hyaluronic acid includes (cross)linked hyaluronic acids. There are widespread examples of modified HA polymers that can form either covalently or physically crosslinked biomaterials and which are frequently used in cosmetics. Common methods for crosslinking hyaluronic acids are known to those skilled in the art, e.g. the most established method is the use of BDDE (butanediol diglycidyl ether) as a cross-linking agent (de Boulle et al., Dermatol Surg 2013; 39:1758-1766) but also other crosslinking agents are used to provide crosslinked HA, e.g., 1,4-butanediol or di-(propan-2,3-diolyl) ether crosslinks. HA-hydrogels based on dynamic covalent coupling (DCC) chemistry are also used.

A “hydrogel” as used herein is a gel of water-insoluble polymer that can bind/contain water (film former) and water. The molecules that make up the gel are chemically, e.g. by covalent or ionic bonds, or physically, e.g. linked by intertwining the polymer chains, linked to form a network, i.e. a hydrogel refers to compositions having a substantially dilute cross-linked system, which exhibits no flow when in the steady-state, although the liquid phase may still diffuse through this system. By weight, gels are mostly liquid, yet they behave like solids because of a three-dimensional cross-linked network within the liquid. It is the crosslinking within the fluid that gives a gel its structure (hardness) and contributes to the adhesive stick. The built-in hydrophilic polymer components swell in water with a considerable increase in volume but without losing their material cohesion. A hydrogel can comprise other protic solvents in addition to water.

When a range (e.g., between a value A and a value B) is given, the value A and value B are within the scope of said range.

“Jasmonic acid compounds” as used herein include jasmonic acid and jasmonic acid derivatives available to one skilled in the art. Such compounds include jasmonic acid, dihydrojasmonic acid, hydroxy jasmonic acid and dihydrohydroxy jasmonic acid, methyl jasmonate and their isomers.

The terms “lotus” as used herein are interchangeable and refer to the lotus flower (nelumbo). There are two species of nelumbo: Nelumbo nucifera Gaertn. (Asian Lotus) and Nelumbo lutea Pear (American Lotus). The roots, fruits, seeds, and stems of all known lotus species are edible. Lotus extracts contain many medicinally useful compounds such as flavonoids, phenolic acids, alkaloids, antioxidants, and vitamin C. Extraction methods for various tissues of lotus are well known to those skilled in the art (see, e.g., Lin et al, “The Latest Studies on Lotus (Nelumbo nucifera)—an Emerging Horticultural Model Plant”, Int J Mol Sci 2019, 20, 3680, 1-13; Chen et al. “Simultaneous Analysis of Anthocyanin and Non-Anthocyanin Flavonoid in Various Tissues of Different Lotus (Nelumbo) Cultivars by HPLC-DAD-ESI-MSⁿ” PLOSone 2013, Vol. 8, Issue 4, 1-13).

“Lotus (root) extracts” as used herein are mostly aqueous extracts or extracts with other protic solvents, preferably lower alcohols (such as ethanol), or mixtures of aqueous and further protic solvents extracts from tissue of lotus plants, such as roots, petals, stems, seeds and/or leaves. The content of a further protic solvent can be from 0% (aqueous extraction) up to 100% of the further protic solvent. Usually, an extract is (freeze) dried after extraction.

“Mannans” are polysaccharides. Vegetable mannans are mostly linear polymers of mannose. The mannose units are predominantly to exclusively β(1-4)-linked. Galactomannans occasionally exhibit α(1-6)-branches with galactose. Glucomannans have a mixed mannose/glucose β(1-4)-skeleton. Galactoglucomannans have a mixed mannose/glucose β(1-4)-skeleton comprising occasional α(1-6)-branches with galactose. Yeast mannans usually display an α(1-6)-skeleton with α(1-2)- and α(1-3)-branches with glucose. There are various sources of galactomannans. A plant source of (1-4)-β-D-manno-pyranose in linear chains branched by (1-6)-linkages with α-D-galactopyranose units in an approximately 3:1 ratio (around 75% mannose units and around 25% Galactose units) is Caesalpinia spinosa gum. Other plant sources for galactomannan, possibly with different mannose to galactose ratios, are Cyamopsis tetragonoloba (guar) or Ceratonia siliqua (carob tree).

The term “oral fortifying polyol” can be a sugar alcohol, disaccharides, a polysaccharide, and preferably a non-reducing sugar. Sugar alcohols belong to a class of polyols that can be obtained by the hydrogenation of sugar compounds having the formula (CHOH)ₙH₂, where higher values are present such as isomalt, maltitol, lactitol, maltitol, maltotetratol, polyglycitol, or combinations thereof. Preferably n is in the range from 7 (inclusive) to 12 (inclusive). Non-reducing sugars belong to a class of saccharides that do not form compounds with an aldehyde or ketone functional group. Non-reducing sugars are stable in water and do not react with weak oxidants to produce sugar alcohols. Non-reducing sugars cannot donate electrons to other molecules, and are typically di-, tri-, tetra-, pentasaccharides such as sucrose, trehalose, raffinose, stachyose, verbascose, or combinations thereof.

“Oral care composition” as used herein means a product that is retained in the oral cavity for a period sufficient to contact tooth surfaces or oral tissues.

The word “or” when used in conjunction of two or more elements means the elements can occur individually and in combination with each other, e.g., “gum or teeth” does not exclude the combination of “gum and teeth”, i.e. the term comprises “only gum”, “only teeth” or “gum and teeth”.

The term “orally acceptable carrier material” includes one or more acceptable solid or liquid excipients or diluents suitable for use in the oral cavity.

As used herein, the term “promoting” means to promote and/or enhance the gum health benefits which are associated with the application of the oral care composition of the present invention to the oral cavity.

The term “protic solvent” is known to the person skilled in the art. In regard of the present invention, it refers to solvents in their liquid phase under standard ambient temperature and pressure (T=298, 15 K and p=1013 hPa) having a hydrogen atom bound to an oxygen, a nitrogen or fluoride. Non-limiting examples are (besides water) short-chained mono or polyhydric alcohols (e.g. methanol, ethanol, 1-propanol, 2-propanol, 1,2-propanediol (propylene glycol), (propane-1,2,3-triol) (glycerol), 1-butanol, etc.), short-chained primary and secondary amines, short-chained primary and secondary amides (e.g. formamide) and furthermore pyridine 3-carboxamide, and polysorbates (e.g. TWEEN 20 (Polyethylenglycol-sorbitan-monolaurate), TWEEN 40 polyoxyethylen-sorbitan-monopalmitate, and TWEEN 80 (Polyethylenglycol-sorbitan-monooleate).

“Short chained” as used herein refers to compounds having a chain of 1, 2, 3, 4, 5, or 6 carbon atoms. Such a chain can be branched and can form a circle and it can be substituted with one or more functional groups (e.g. —OH for alcohols, COOH for organic acids, —NH₂ for amines, C(O)NH₂ for amides.

As used herein, the term “strip” includes a material 1) whose longest dimensional length is generally greater than its width, and 2) whose width is generally greater than its thickness. Strips can be rectangular, curved, curved, semi-circular, have rounded corners, cut slits, cut notches, bent into three-dimensional shapes, or combinations thereof. Strips can be solid, semi-rigid, textured, malleable, flexible, deformable, permanently deformable, or combinations thereof. Strips can be made from sheets of plastic, including polyethylene, or sheets of wax.

As used herein, the term “substantially free” refers to the presence of less than 0.001% (w/w) of a specified material in a composition. Also included by this term are compositions wherein the specified material is present only as an impurity of one of the other materials which were intentionally added.

The term “essentially free “free” refers to the presence of less than 0.0001% (w/w) of a specified material in a composition.

The term “free” means that the indicated material is not present at analytically detectable levels in a composition.

The term “tooth” as used herein refers to both a natural tooth and an artificial tooth and includes one tooth or more teeth.

The term “tooth surface” as used herein refers to both natural tooth surface(s) and artificial tooth surface(s) or denture surface(s).

The term “total water” or “total solvent” as used herein means both free water and water bound by other ingredients in the oral care composition, or free solvent and solvent bound by other ingredients in the oral care composition, e.g., by mucoadhesive polymers in a hydrogel.

A “water-insoluble polymer” as used herein can form a (hydro)gel with water and/or other solvents. It is hardly or not at all soluble in water at 25° C. In this context, “barely” means that a maximum of 10% of a polymer can be dissolved in water at 25° C. without heating the water and/or the polymer. Non-limiting examples are gelatin (a mixture of animal proteins and collagen), (cross-linked) poly(meth)acrylates such as (cross-linked) polyacrylic acids, (cross-linked) polyurethane polymers, (trimethylated) hyaluronic acid, povidone iodine, and polysaccharides and their derivatives, e.g., mannans, glycogen, starch, alginates, pectin, chitin, chitosans (deacetylated chitin), and cellulose and its derivatives. The cellulose derivatives with mucoadhesive properties are mainly (cross-linked) Na carboxymethyl cellulose, hydroxyethyl cellulose, ethyl cellulose, methyl cellulose and hydroxypropylmethyl cellulose. The crosslinking of poly (meth) acrylates e.g., during the polymerization by adding compounds that have at least two vinyl substructures such as divinyl glycol or pentaerythritol allyl ether.

A “vasodilatation component” results in a physiological reaction that causes the vascular cross-section to increase and thus the blood flow behind the affected vascular section to increase.

“Viscosity” as used herein is the property of a liquid to resist the mutual displacement of two adjacent layers. Viscosity is given in pascal seconds. The viscosity of a composition according to the invention can be determined by a rotational rheometer with a cone-plate with a std smooth surface (usually a stainless-steel surface) 4°/40 mm diameter.

The “Yield point” in viscosity refers to the resistance of initial flow of the fluid or in other words, the stress required to start the movement of the fluid.

“Zeatin compounds” as used herein include cis and trans isomers of zeatin and the cis and trans isomers of zeatin derivatives available to one skilled in the art.

Unless otherwise stated, percentages are given as percentages by weight (% (w/w)). Unless otherwise indicated, weight percentages (% (w/w)) and ratios are based on the weight of the total composition. All ingredient percentages, ratios, and amounts stated herein are based on the actual amount of the compounds of interest and do not include solvents, fillers, or other materials with which the ingredient may be combined as a commercial product, unless otherwise noted.

The term “comprising” as used herein in combination with a subject matter means also other subject matters can be encompassed/present. The term “comprising” also includes the embodiment “consisting of” as one preferred choice. For example, a composition comprising components (a) to (d) can also have further components. However, this term also encompasses the preferred embodiment that said composition consists of components (a) to (d), i.e., no further components are present. Thus, if the term “comprising” is used herein, it is always possible to limit a subject matter comprising essential subject matters and further subject matter into the embodiment said subject matter consists of said essential subject matter (and no further subject matter can be added).

The skilled person will understand that any two or more embodiments of the invention, irrespective of the fact if they are embodiments, preferred embodiments, more preferred embodiments etc., can be combined with each other unless such a combination would contradict a law of nature or is explicitly excluded.

### DETAILED DESCRIPTION OF THE INVENTION

The oral care composition described in the present invention is designed for application to the gum tissue as well as other soft tissues (e.g., buccal mucosa) and teeth in a user's oral cavity, as well as for application to a user's aligner splint.

Surprisingly, it has been found that an oral care composition of the present invention preferably with optimal viscosity provides better sensory benefits to users and is particularly useful for promoting gum health. It was surprisingly found that an oral care composition according to the invention with an optimum viscosity, resulting from HA and preferably a second water insoluble polymer, in combination with anti-inflammatory components and preferably further protective components such as an antioxidant, preferably from the group of catechin, is excellently suited to greatly improving the health of the gum and the mouthfeel (taste in the mouth, hypersensitivity, bad breath and dry mouth) of wearers of aligner splints during the period of tooth position correction.

Aspect one of the present invention relates to an oral care composition comprising:
(a) at least one protic solvent, wherein at least one protic solvent is water, and wherein the amount of the sum of all protic solvents is between 45% (w/w) and 85% (w/w), and the amount of water is at least 45% (w/w);
(b) at least one water-insoluble polymer, wherein at least one water-insoluble polymer is hyaluronic acid, and wherein the amount of the sum of all water-insoluble polymers is between 0.4% (w/w) and 5% (w/w), the amount of the sum of all water-insoluble polymers is preferably between 0.8% (w/w) and 5% (w/w), and wherein the amount of hyaluronic acid is at least 0.4% (w/w), preferably at least 0.8% (w/w), more preferably, the amount of the sum of all water-insoluble polymers is between 0.9% (w/w) and 5% (w/w), and wherein the amount of hyaluronic acid is at least at least 0.8% (w/w);
(c) one or more sugar alcohol (polyol) selected from the group consisting of sorbitol, xylitol, erythritol, wherein the amount of the sum of the polyol(s) (selected from the group consisting of sorbitol, xylitol, erythritol, and a combination of two or three of the said three polyols) is between 10% (w/w) and 50% (w/w); in one preferred embodiment, the polyol is xylitol, wherein the amount of xylitol is between 10% (w/w) and 50% (w/w); in another preferred embodiment, the polyol is erythritol, wherein the amount of erythritol is between 10% (w/w) and 50% (w/w);
(d) at least one other anti-inflammatory component, in addition to b) and c), selected from a group consisting of alpha-bisabolol (6-methyl-2-(4-methylcyclohex-3-en-1-yl)hept-5-en-2-ol), chamazulene (7-ethyl-1,4-dimethylazulene), matricin ([9-hydroxy-3,6,9-trimethyl-2-oxo-3,3a,4,5,9a,9b-hexahydroazuleno[4,5-b]furan-4-yl]acetate), curcumin (1,7-Bis(4-hydroxy-3-methoxyphenyl)hepta-1,6-diene-3,5-dione), beta-caryophyllene (4,11,11-trimethyl-8-methylidenebicyclo[7.2.0]undec-4-ene), eugenol (2-methoxy-4-prop-2-enylphenol), camphor (1,7,7-trimethylbicyclo[2.2.1]heptan-2-one), cineole (1,3,3-trimethyl-2-oxabicyclo[2.2.2]octane), cuminaldehyde (4-isopropylbenzaldehyde), thujone (4-methyl-1-propan-2-ylbicyclo[3.1.0]hexan-3-one), chlorhexidine (1,6-bis(4-chloro-phenylbiguanido)hexane), povidone iodine (1-ethenylpyrrolidin-2-one), cetylpyridinium chloride or bromide (1-hexadecylpyridinium chloride or bromide), and ursolic acid, wherein the amount of the sum of all others anti-inflammatory components (d) is between 0.001% (w/w) and 2% (w/w);
wherein the sum of (a), (b), (c), and (d) must be between 90% (w/w) and 100% (w/w).

In other words, a composition according to the invention can comprise up to 10% (w/w) further components which may be added intentionally (e.g., for enhancing the fluidity or adjusting the pH of a composition) or which are added together with any of the components (a) to (d), e.g. auxiliaries in a carbomer product or components in a plant oil or extract except of a component (a), (b), (c) or (d).

In one preferred embodiment, a composition comprises groups (a), (b), (c), and (d) as defined in aspect one and any of the embodiments disclosed herein, and at least one or more antioxidants selected from the group consisting of L-ascorbic acid or derivatives thereof, dibutyl hydroxy toluene, butyl hydroxy anisole, superoxide dismutase, carotenoids, astaxanthin, rutin or derivatives thereof, hesperidin, quercetin, catechin; gallic acid or derivatives thereof, allantoin (N-(2,5-Dioxoimidazolidin-4-yl)urea) and derivatives thereof, glutathione or derivatives thereof, glutathione, β-carotenes or derivatives thereof, ubiquinol, polyphenols, sweet hydrangea leaf, turmeric, rose fruit, echinacea, Scutellaria root, Hypericum erectum, Chinese gall nut, Geranium thunbergii, rice, rice bran, comfrey, Xanthoxylum piperitum, labiate, peony root, soybean, “Natto” (soybeans fermented in their own bacteria), tea leaf, clove, loquat, peony, horse chestnut, saxifrage, rooibos, rosemary, spirulina, chlorella and dunaliella, bilirubin, cholesterol, tryptophan, histidine, thiotaurine and hypotaurine, wherein the amount of the sum of the said one or more antioxidant(s) is between 0.001% (w/w) and 5% (w/w); and wherein the sum of (a), (b), (c), (d) and the one or more antioxidant is between 90,001% (w/w) and 100% (w/w).

Another preferred embodiment refers to a composition according to aspect one, consisting of group (a), (b), (c), and (d) as defined in aspect one and any of its embodiments and one or more further component(s) (e), wherein the amount of the sum of all further components (e) is 10% (w/w) or less, preferably, wherein the further components (e) are selected from the group consisting of (e1) extracts and oils, (e2) buffers, (e3) chelating agents, (e4) antioxidants, (e5) preservatives, (e6) humectants, (e7) flavorings, (e8) vitamins, (e9) retinoids, (e11) amino acid, (e12) oral cavity fortifying polyols, (e13) hydroxy acids, (e14) fluoride, (e15) sweeteners, (e16) colorants, (e17) peptides, (e18) cooling agents, (e19) warming agents, (e20) tingling agents, (e21) emulsifier, and (e22) further additives; also preferably, wherein further components (e) comprise at least one component (e) selected from a group consisting of propolis (e1), lotus root extract (e1) and catechins (e4), wherein the amount of lotus root extract is between 0.1% (w/w) and 5% (w/w) and the amount of catechins is between 0.001% (w/w) and 5% (w/w), more preferably between 0.2% (w/w) and 5% (w/w) and the amount of propolis is between 0.01% (w/w) and 2% (w/w) (in addition to the supply of vitamins and other important substances, such as antioxidants, necessary for gum health, the presence of lotus root extract has the advantage that the acceptance of the composition by the user is increased due to the cocoa-like taste and, thus, the effect of the composition is improved by a more regular use of the composition by the user. Catechins are vasodilation components that leads to an improvement in blood circulation in the gum; propolis is a mass produced by bees with antibiotic, viral and mycotic effects and is rich in essential oils, flavonoids, phenols and polysaccharides);

wherein the sum of (a), (b), (c), (d), and (e) must always add up to 100%.

In one preferred embodiment, the present invention is directed to an oral care composition consisting of (a), (b), (c), (d) as defined in aspect one and the embodiments disclosed herein and further components (preferably components (e)); and wherein the further components (preferably components (e)) comprises at least one or more antioxidant(s) (e4), preferably, wherein the amount of the sum of the said one or more antioxidant(s) is between 0.001% (w/w) and 5% (w/w); and optionally comprises further components, preferably selected from groups (e1) to (e3) and (e5) to (e22);

wherein the sum of (a), (b), (c), (d) is between 90% (w/w) and 99,999% (w/w), the sum of (e4) is between 0.001% (w/w) and 5% (w/w) and the sum of all further components (preferably components (e)) is between 0% (w/w) and 9,999% (w/w), and the sum of (a), (b), (c), (d), (e4) and all further components, preferably selected from groups (e1) to (e3) and (e5) to (e22), must always add up to 100%.

In one preferred embodiment, a composition comprises one or more antioxidants selected from the group consisting of ascorbic acid, Na ascorbyl phosphate, catechin, allantoin or its allantoinate form, and limonene, more preferably selected from the group consisting of Na ascorbyl phosphate, C, EC, ECG, EGC and EGCG, allantoin, allantoinate and limonene, even more preferably selected from the group consisting of C, EC, ECG, EGC and EGCG.

In another preferred embodiment, the oral care composition is essentially free of triclosan (5-chloro-2-(2,4-dichlorophenoxy)phenol) (i.e. the amount of triclosan is 0.0001% (w/w) or less, more preferably 0.000001% (w/w) or less), most preferably free of triclosan (i.e. triclosan is not present at analytically detectable levels in a composition, a composition does not comprise triclosan).

Preferably, the oral care composition is in a hydrogel form. In one preferred embodiment, the viscosity of the composition is between 10 Pas and 800 Pas at a shear rate of 1 [1/s] and a temperature of 22° C. (preferably measured with a rotational rheometer with a cone-plate with std smooth surface (stainless steel) with 4° and 40 mm diameter). Unless otherwise stated, were measured using a rotary rheometer with a 4°, 40 mm diameter std smooth surface cone-plate at a temperature of 22° C. (±2° C.). A viscosity of less than 10 [Pas] at a shear rate of 1 [1/s] is an indication that the composition between the splint and the gums/teeth of a user, like a mouthwash, is removed from the gum or teeth surface too quickly to be able to have a long-lasting effect. At viscosities above 800 [Pas] at a shear rate of 1 [1/s], a constant quantity of a composition no longer emerges from a dispenser, and it is difficult to evenly distribute such a composition to the gum, teeth or a splint surface. The viscosity is particularly preferably in the range between 20 [Pas] and 500 [Pas] at a shear rate of 1 [1/s], more preferably between 20 [Pas] and 200 [Pas], even more preferably between 40 [Pas] and 200 [Pas], such as (60±20 [Pas]). It is also preferred if the yield point of a composition according to the invention is 2 Pa or more at a shear rate of 0 [1/s], more preferably in the range between 2 Pa and 100 Pa, even more preferably in the range from 10 Pa to 40 Pa. It was observed the presence of 0.1% HA is not sufficient to form a complete gel form. While 0.3% HA already show a better gel behavior, a composition with this concentration of HA with no further polymer still acts partly more like a Newtonian fluid and does not show the same good adhesive effect compared to a formulation with at least 0.4% (w/w) water-insoluble HA and, preferably, a further water-insoluble polymer. Thus, a composition comprises preferably at least 0.4% (w/w) HA, more preferably at least 0.8% (w/w) water-insoluble polymer (wherein at least 0.4% (w/w) are HA). The viscosity profile range, due to the use of HA and preferably a second water-insoluble polymer, offers the user a better sensory experience of spreadability. If a product is too viscous, it would be difficult to spread evenly onto the gum tissue. If the product has a too low viscosity, the exposure time is reduced, e.g., by enhanced salivation reduction of the composition in the oral cavity. A hydrogel comprising water and at least one water-insoluble polymer can and may need to comprise other substances, such as buffers (e.g., tris(hydroxymethyl)aminomethane (TRIS)), salts, complexing agents (e.g. EDTA disodium salt), and other solvents (e.g. propylene glycol).

In a preferred embodiment, the pH of an oral care composition is between pH 5.5 and pH 8, more preferably between pH 6 and pH 8, even more preferably between pH 6 and pH 7.2 or between pH 6.5 and pH 7.8, such as between pH 6.8 and pH 7.8, or even between pH 7.0 and pH 7.8.

The compositions according to the present invention should not comprise solid particles (abrasives) which would scrub against and, thus, irritate the gum, especially, when a composition is used together with a splint. The skilled person understands that abrasives used in oral care products (such as toothpastes) are either not soluble in a oral care composition or present in a concentration which is higher than the solubility concentration of said abrasive. For example, sodium bicarbonate (NaHCO₃) is a well-known abrasive in toothpastes. The solubility of NaHCO₃ in water at 20° C., 1013 mbar and pH 7 (in the following SO) is 93.4 g/l (93.4 g/1000 g H₂O=9.34% (w/w)), i.e., a composition according to the invention may comprise dissolved (solubilized) NaHCO₃ but only in a concentration less than 9.34% (w/w). Tricalcium phosphate (Ca₃(PO₄)₂) has a SO of 0.02 g/l (0.002% (w/w)), i.e., a composition according to the invention could comprise dissolved (solubilized) (Ca₃(PO₄)₂) but only in a concentration less than 0.002% (w/w), while calcium hydroxyapatite (Ca₁₀(PO₄)₆(OH)₂) is not soluble in water. Not soluble (not SO) as used herein means a solution comprises less than 0.0001%/w/w) of a component. In one preferred embodiment, a composition does not comprise solid particles, preferably no solid particles selected from the group consisting of calcium phosphates such as calcium pyrophosphate (Ca₂O₇P₂/not SO), dicalcium phosphate (or calcium hydrogen phosphate) (CaHPO₄/SO: 0.1 g/l), tricalcium phosphate (or calcium phosphate or tricalcium orthophosphate) (Ca₃(PO₄)₂/SO: 0.02 g/l), calcium metaphosphate (cyclic condensation products ortho-phosphoric acid (H₃PO₄), e.g., calcium trimetaphosphate has a 6 membered ring system consisting of 3 oxygen and 3 phosphorous atoms (SO: less than 0.02 g/l)), calcium polyphosphate (non-cyclic condensation products of ortho-phosphoric acid (SO: less than 0.02 g/l)), or calcium hydroxyapatite (Ca₁₀(PO₄)₆(OH)₂/no SO); calcium or strontium carbonate (CaCO₃/SO: 0.014 g/l; or SrCO₃ (SO: 0.01 g/l)); sodium bicarbonate (also hydrogen carbonate—NaHCO₃/SO: 93.4 g/l): silicas such as fused silica (SIO₂/SO: 0.01 g/l), fumed silica (SO: less than 0.12 g/l), precipitated silica (SO: less than 0.12 g/l), hydrated silica (silicon dioxide which has a variable amount of water in the formula, i.e. oxygen acids of silicium H₂ₙ₊₂SiₙO₃ₙ₊₁, wherein n is an integer of 1 or more e.g., Si(OH)₄ wherein Si atoms are bridged by an oxygen/(SO: less than 0.12 g/l)); diatomaceous earth (not SO); barium sulfate (SO: 0.022 g/l); wollastonite (Ca₃[Si₃O₉]/not SO); perlite (not SO), polymethylmethacrylate particles (PMMA/no SO), silicon beads (e.g., cross-linked siloxane particles such as tospearl/SO less than 0.012 g/l), and combinations of any of the foregoing. Abrasives are commercially available, e.g., “Syloid,” or Zeodent®-119, -118, -109, and -129.

In one preferred embodiment, the amount of each component selected from the group consisting of calcium pyrophosphate, calcium hydroxyapatite, diatomaceous earth, wollastonite, perlite, PMMA, silicon beads is, if present, 0.00001% (w/w) or less, most preferably, these components are not present in a composition according to the invention.

In another preferred embodiment, if present, the amount of dicalcium phosphate in a composition is 0.008% (w/w) or less. In another preferred embodiment, if present, the amount of tricalcium phosphate in a composition is 0.0016% (w/w) or less. In another preferred embodiment, if present, the amount of calcium metaphosphate in a composition is 0.0016% (w/w) or less. In another preferred embodiment, if present, the amount of calcium polyphosphate in a composition is 0.0016% (w/w) or less. In another preferred embodiment, if present, the amount of calcium carbonate in a composition is 0.001% (w/w) or less. In another preferred embodiment, if present, the amount of strontium carbonate in a composition is 0.0008% (w/w) or less. In another preferred embodiment, if present, the amount of sodium bicarbonate is 7.4% (w/w) or less. In another preferred embodiment, if present, the amount of fused silica is 0.0008% (w/w) or less. In another preferred embodiment, if present, the amount of fumed silica is 0.0009% (w/w) or less. In another preferred embodiment, if present, the amount of precipitated silica is 0.0009% (w/w) or less. In another preferred embodiment, if present, the amount of hydrated silica is 0.0009% (w/w) or less. In another preferred embodiment, if present, the amount of barium sulfate is 0.0017% (w/w) or less. In another preferred embodiment, if present, the amount of silicon beads is 0.0009% (w/w) or less.

In another preferred embodiment, an oral care composition is substantially free of calcium pyrophosphate, calcium hydroxyapatite, diatomaceous earth, wollastonite, perlite, PMMA, silicon beads, dicalcium phosphate tricalcium phosphate, calcium metaphosphate, calcium polyphosphate, calcium carbonate, strontium carbonate, fused silica, fumed silica, precipitated silica, hydrated silica, silicon beads, and barium sulfate, i.e. the amount of any of these components in a composition is 0.001% (w/w) or less. This is irrespective of the form (solubilized or solid) of any of the components.

In another preferred embodiment, an oral care composition is substantially free of calcium pyrophosphate, calcium hydroxyapatite, diatomaceous earth, wollastonite, perlite, PMMA, silicon beads, dicalcium phosphate tricalcium phosphate, calcium metaphosphate, calcium polyphosphate, calcium carbonate, strontium carbonate, fused silica, fumed silica, precipitated silica, hydrated silica, silicon beads, and barium sulfate, i.e. the amount of any of these components in a composition is 0.001% (w/w) or less. This is irrespective of the form (solubilized or solid) of any of the components.

More preferably, a composition is a composition, wherein the amount of each component selected from the group consisting of calcium pyrophosphate, calcium hydroxyapatite, diatomaceous earth, wollastonite, perlite, PMMA, silicon beads is, if present, 0.00001% (w/w) or less; the amount of dicalcium phosphate, if present, is 0.008% (w/w) or less; the amount of each component selected from the group consisting of tricalcium phosphate, calcium metaphosphate, and calcium polyphosphate is 0.0016% (w/w) or less; the amount of calcium carbonate is 0.001% (w/w) or less; the amount of strontium carbonate in a composition is 0.0008% (w/w) or less; the amount of fused silica is 0.0008% (w/w) or less; the amount of each component selected from the group consisting of fumed silica, precipitated silica, hydrated silica, and silicon beads is 0.0009% (w/w) or less; the amount of barium sulfate is 0.0017% (w/w) or less, even more preferably, a composition is free of any of these components, i.e. does not comprise any of these components. In another preferred embodiment, a composition is a composition, wherein the amount of sodium bicarbonate is 7.4% (w/w) or less, preferably 1% (w/w) or less.

### Protic Solvent (a)

A protic solvent (a) in regard of the present invention is a solvent selected from the group consisting of water, short-chained mono or polyhydric alcohols (e.g. methanol, ethanol, 1-propanol, 2-propanol, 1,2-propanediol (propylene glycol), (propane-1,2,3-triol) (glycerol), 1-butanol, etc.), short-chained primary and secondary amines, short-chained primary and secondary amides (e.g. formamide) and furthermore pyridine 3-carboxamide, and polysorbates (e.g. TWEEN 20 (Polyethylenglycol-sorbitan-monolaurate), TWEEN 40 polyoxyethylen-sorbitan-monopalmitate, and TWEEN 80 (Polyethylenglycol-sorbitan-monooleate). It is particularly preferably an orally acceptable protic solvent. The skilled person is aware that the sum of all protic solvents in a composition cannot be less or more than the possible minimal or maximal amount, respectively, of all protic solvents in a composition according to the invention (e.g., the sum of water and 1,2-propylene glycol (propane-1,2-diol), 1,3-butylene glycol (butane-1,3-diol) or 1,2-pentylene glycol (pentane-1,2-diol) if those are the two solvents in a composition in accordance with the invention, cannot be less than 45% or more than 85% (w/w)).

In one preferred embodiment, (a) consists of water. In another preferred embodiment, (a) comprises water and one or more further protic solvent(s). In another preferred embodiment, (a) consists of water and one or more protic solvent(s) (which are preferably solvent(s) in their liquid phase under standard ambient temperature and pressure (T=298, 15 K and p=1013 hPa) and have a hydrogen atom bound to an oxygen, a nitrogen or fluoride, more preferably to an oxygen or nitrogen). In yet another preferred embodiment, (a) consists of water and at least one further protic solvent and the amount of water is at least 50% (w/w), more preferably between 55% (w/w) and 85% (w/w), such as between 55% (w/w) and 75% (w/w), more preferably between 55% (w/w) and 70% (w/w), even more preferably between 55% (w/w) and 68% (w/w). In another preferred embodiment, (a) comprises water and propylene glycol. In yet another preferred embodiment, (a) consists of water and propylene glycol. In another preferred embodiment, (a) comprises water and pentane-1,2-diol. In yet another preferred embodiment, (a) consists of water and pentane-1,2-diol. In another preferred embodiment, (a) comprises water and ethanol. In another preferred embodiment, (a) consists of a mixture of water and ethanol. In another preferred embodiment, (a) comprises water and glycerol. In another preferred embodiment, (a) consists of water and glycerol. In another preferred embodiment, (a) comprises water, 1,2-propylene glycol and ethanol (more preferably consists of these three components) and the amount of water is between 50% (w/w) and 76% (w/w), the amount of 1,2-propylene glycol is between 2% (w/w) and 6% (w/w) and the amount of ethanol is between 0.5% (w/w) and 3% (w/w), wherein the sum of all protic solvents (a) in the composition is at most 85% (w/w). In another preferred embodiment, (a) comprises, more preferably consists of, water and one or more one or more C₁-C₅ alcohols (e.g., ethanol, 1- or 2-propanol, 1,2-propylene glycol, 1,2-pentylene glycol, and glycerol), more preferably C₁-C₄ alcohols (e.g., mono- or diols such as ethanol, 1- or 2-propanol, 1,2-propandiol, 1- or 2-butanol, or glycerol), even more preferably C₁-C₃ alcohols (e.g., ethanol, 1- or 2-propanol, 1,2-propandiol, or glycerol). In a further preferred embodiment, (a) comprises water and a further protic solvent, preferably propylene glycol. The amount of the further protic solvent, preferably 1,2-propylene glycol, is preferably between 1.5% (w/w) and 20% (w/w), more preferably between 3% (w/w) and 20% (w/w), even more preferably in a range between 3% (w/w) and 10% (w/w). In another preferred embodiment, (a) comprises water and another protic solvent, preferably propylene glycol, and the amount of water is between 50% (w/w) and 70% (w/w) (e.g. 57% (w/w) to 67% (w/w)) and another protic solvent, preferably 1,2-propylene glycol, and the amount of the further protic solvent, preferably 1,2-propylene glycol, is between 3% (w/w) and 8% (w/w) (e.g. around 5.9% (w/w) such as between 5.3% (w/w) and 6.4% (w/w)). In a particularly preferred embodiment, (a) consists of water and 1,2-propylene glycol. In another preferred embodiment, (a) comprises water and 1,2-pentylene glycol and ethanol (more preferably consists of these three components) and the amount of water is between 50% (w/w) and 76% (w/w), the amount of 1,2-pentylene glycol is between 2% (w/w) and 6% (w/w) and the amount of ethanol is between 0.5% (w/w) and 3% (w/w)), wherein the sum of all protic solvents in the composition is at most 85% (w/w). In yet another preferred embodiment, (a) comprises, more preferably consists of, water and at least one further protic solvent selected from the group consisting of 1,2-propylene glycol, 1,3-butylene glycol, 1,2-pentanediol, glycerol and ethanol and the amount of water is between 50% (w/w) and 66% (w/w), the amount of 1,2-propylene glycol is between 1% (w/w) and 5% (w/w), the amount of 1,3-butylene glycol is between 1% (w/w) and 5% (w/w), the amount of 1,2-pentylene glycol is between 1% (w/w) and 6% (w/w), the amount of glycerol is between 1.5% (w/w) and 15% (w/w), and the amount of ethanol is between 0.5% (w/w) and 5% (w/w), more preferably between 0.5% (w/w) and 3% (w/w), wherein the sum of all protic solvents in the composition is at most 85% (w/w).

In another preferred embodiment, (a) consists of a mixture of water and two or three other protic solvents, preferably selected from the group consisting of ethanol, 1,2-propylene glycol, 1,2-pentalediol and glycerol, e.g., ethanol and 1,2-propylene glycol and/or 1,2-pentanediol. In another preferred embodiment, (a) consists of a mixture of water and three other protic solvents, preferably selected from the group consisting of glycerol, ethanol 1,2-pentylene glycol and 1,2-propylene glycol. This can be the case, e.g., by using carbomer gels, which often contain 1,2-propylene glycol in addition to water and the use of an ethanolic plant extract (e.g. chamomile) or an ethanolic propolis product in the production of a composition in accordance with the invention. Glycerol and 1,2-pentanediol can be added to improve the haptic properties and stability of a composition. Moreover, alcohols such as ethanol support the formation of a gel when combining water and hyaluronic acid, e.g., a Na salt of hyaluronic acid.

In another preferred embodiment, (a) comprises, more preferably consists of, water and at least one solvent selected from the group consisting of mono- or polyhydric C1, C2, C3, C4, C5, or C6 alcohols (preferably methanol, ethanol, 1-propanol, 2-propanol, 1,2-ethylene glycol, 1,2-propylene glycol, 1,3-butylene glycol, 1,2-pentylene glycol, 1,2-hexylene glycol, 1,2,3-propanetriol (glycerol), 1-butanol, 2-butanol, 2-methyl-1-propanol, 2-methyl-2-propanol), C1, C2 or C3 carboxylic acids (formic acid, acetic acid, propionic acid, preferably formic acid or acetic acid, more preferably acetic acid), and a combination of any of the foregoing; more preferably, a protic solvent in addition to water is at least one solvent selected from the group consisting of ethanol, 1-propanol, 2-propanol, 1,2-propylene glycol, 1,3-butylene glycol, glycerol, 1,2-pentylene glycol, acetic acid, or a combination of any of the foregoing. In yet another embodiment, the protic solvent (a) consists of water. In another preferred embodiment, the protic solvent (a) consists of a combination of water with one or more of solvents selected from the group consisting of ethanol, 1-propanol, 2-propanol, 1,2-propylene glycol, glycerol, 1,3-butylene glycol, and 1,2-pentylene glycol.

In a more preferred embodiment, (a) comprises at least 60%, more preferably at least 98%, even more preferably at least 99%, such as at least 99.9% (based on the weight of the sum of all protic solvents in a composition) water or a combination of water and one or more protic solvent selected from the group consisting of C2- and C3-chained mono alcohols (e.g. ethanol, 1-propanol, 2-propanol), short-chained polyhydric alcohols (e.g., 1,2-propylene glycol, 1,3-butylene glycol, 1,2-pentylene glycol, and glycerol), polysorbates (e.g. TWEEN 20, TWEEN 40, and TWEEN 80), formic acid, acetic acid and primary amides (e.g. pyridine-3-carboxamide), even more preferably selected from the group consisting of ethanol, 1-propanol, 2-propanol, 1,2-propylene glycol, 1,3-butylene glycol, 1,2-pentylene glycol, glycerol, TWEEN 20, TWEEN 40, TWEEN 80, acetic acid and pyridine-3-carboxamide; or any of the other preferred protic solvent combinations disclosed herein (the skilled person will understand that traces of other protic solvents, due to being auxiliaries or residues of, e.g., extraction products (see, e.g., turmeric extraction media below) or water-insoluble polymer products, may be present in a composition according to the invention). In another preferred embodiment, the amount of water is at least 70% (based on the weight of the sum of all protic solvents in a composition). In a further preferred embodiment, the amount of water is at least 80% (based on the weight of the sum of all protic solvents in a composition) and the amount of any one of the further protic solvents ethanol, 1-propanol, 2-propanol, 1,3-butylene glycol, 1,2-propylene glycol, 1,2-pentylene glycol, acetic acid, and pyridine-3-carboxamide (if present) is each independently from each other 5% or less (based on the weight of the sum of all protic solvents in a composition), the amount of glycerol (if present) is 15% or less (based on the weight of the sum of all protic solvents in a composition), and the amount of TWEEN 20, TWEEN 40 and TWEEN 80 (if present) is each independently from each other 0.01% or less (based on the weight of the sum of all protic solvents in a composition).

More preferably, the amount of 1-propanol, 2-propanol, acetic acid and pyridine-3-carboxamide is independently from each other 1% or less based on the total sum of all protic solvents in a composition. The skilled person will understand that the sum of the weight of all protic solvents (a) in a composition will always have to sum up to 100% if based on the sum of all protic solvents in a composition while the total amount of (a) will always be in the range between 45% (w/w) and 85% (w/w) based on the sum of all components (a), (b), (c), (d), and (optionally) (e) of a composition according to the invention.

### Water-Insoluble Polymer (b)

At least one water-insoluble polymer (b) is comprised in an oral care composition. Group (b) comprises at least hyaluronic acid (HA) (including any one of its sodium (Na), lithium (Li), potassium (K), magnesium (Mg) and calcium (Ca) salts, thiolated forms of any of the foregoing (e.g., HA-cysteine ethyl ester) or crosslinked forms (e.g. urea-crosslinked HA or any of the crosslinked HAs resulting from the reactions mentioned under definition. Crosslinked HA or salts thereof is commercially available).

In addition to the ability to bind large amounts of water (up to 6 liters/g HA), HA also has mucoadhesive properties. A water-insoluble, mucoadhesive polymer, in particular in the form of a hydrogel, can be used for a prolonged retention time of further components of an oral care composition according to the invention on the surface of the oral cavity, such as the gum. HA is known to be an indispensable component of intact, healthy gum and oral mucosal tissue and it induces early formation of granulation tissue, inhibits inflammation, promotes epithelial turnover and also promotes angiogenesis of connective tissue.

In one preferred embodiment, HA, such as HA or its Na salt or an alkaline earth salt (preferably selected from a Mg salt or Ca salt, more preferably a Mg salt), has a molar mass between 5 kDa and 2000 kDa, preferably between 5 kDa and 200 kDa, e.g., between 8 kDa and 12 kDa, between 10 kDa and 50 kDa; or between 201 kDa and 2000 kDa, e.g., between 1200 kDa and 1800 kDa); or a combination of HA polymer with molar masses between 5 kDa and 2000 kDa such as combinations of at least two HA polymers selected from the group consisting of polymers with molar masses between 5 kDa to 200 kDa, and between 160 kDa and 2000 kDa.

Preferably, a HA (such as a salt thereof preferably Na salt thereof or Mg salt thereof) used in the present invention has a molecular weight (MW) between 1 kDa and 5000 kDa, preferably between 5 kDa and 2000 kDa, such as between 2 kDa and 500 kDa, more preferably a low molecular weight HA (or a salt thereof such as Na salt thereof) between 5 kDa and 200 kDa, such as between 5 kDa and 150 kDa, or between 5 kDa and 60 kDa, or 7 kDa and 55 kDa. The HA can be linear polymers, crosslinked polymers or mixtures of linear and crosslinked polymers, HA or a salt of HA, preferably a Na salt of HA, e.g., Hyamax (Na hyaluronate with a molar mass of 8 kDa to 12 kDa). Low-molecular HAs (such as their salts) have the advantage to easily penetrate the (mucous) membrane. This applies in particular to HAs having a molecular weight below 200 kDa, such as e.g. below 150 kDa. In a preferred embodiment, HA is present as a salt, preferably its Na salt. In another embodiment, HA is a thiolated HA or a salt, preferably a Na salt, thereof, e.g., HA-cysteine ethyl ester conjugates (or their salts). In a further preferred embodiment, HA is a HA-cysteine ethyl ester conjugate or a salt, preferably a Na salt, thereof. In a further preferred embodiment, at least one HA in a composition according to the invention is an alkali salt (preferably a Na, Li or K salt, more preferably a Na salt; or Mg salt) of HA (preferably between 5 kDa and 2000 kDa, more preferably between 5 kDa and 200 kDa, e.g., between 8 kDa and 12 kDa, between 10 kDa and 50 kDa; or between 201 kDa and 2000 kDa, preferably between 1200 kDa and 1800 kDa.

In another preferred embodiment, at least one HA in a composition according to the invention is a cross linked HA. In yet another preferred embodiment, the HA are cross linked at a hydroxy group, the NHCOCH₃ group or the carboxylic group. The skilled person is aware of crosslinking agents for the carboxylic group. Preferred examples for the modification of a in a HA molecule are dicyclohexyl carbodiimide, N-hydroxysuccinimide (NHS), Benzotriazol-1-yl-oxy-tris-(dimethylamino)-phosphonium hexafluorophosphate, 1 hydroxy·7·azobenzotriazole (HOAt), carbonyl diimidazole, 2-(1H-benzotriazol-1-yl)-1,1,3,3-tetramethyluronium hexafluorophosphate (HBTU) and 1-ethyl-3-(3-dimethylaminopropyl)carbodiimide hydrochloride (EDC). Preferred examples for the modification of a hydroxy group (—OH) within a HA molecule are 1,4-butanediol-diglycidyl-ether (BDDE), divinyl sulfone (DVS), glutaraldehyde, cyanogen bromide, octyl succinic anhydride, acid chloride, methacrylic anhydride and Na periodate. Further crosslinked HAs can be achieved by using, e.g., aldehyde-functionalized HA (e.g. based on the introduction of a double bond in the glucuronic acid of the hyaluronic acid (HA), by the biodegradation of HA with hyaluronate lyase, followed by the generation of a free aldehyde group at the nonreducing end of hyaluronic acid via ozonolysis and the subsequent reduction of the generated ozonide, see, e.g., Ruhela et al in Bioconjugate Chem. 2006, 17, 5, 1360-1363). The resulting aldehyde-functionalized HA is then coupled with a reaction partner selected from the group consisting of methacrylate chitosan, cystamine dihydrochloride, carboxyethyl-modified chitosan, hydrazide-functionalized poly (γ-glutamic acid), collagen, N-succinyl-chitosan, carbohydrazide-modified gelatin, amino-modified HA (each time DCC based on Schiff base), hydrazine-modified elastin-like protein, hydrazide-modified HA (each time DCC based on hydrazone), adipic dihydrazide-modified HA and dialdehyde modified-pectin, hydrazide modified HA and benzaldehyde terminated F127 triblock polymers, hydrazine-modified HA and aldehyde-modified HA and catalysts, carbohydrazide-modified carboxymethyl cellulose and aldehyde-modified HA, gallol and hydrazide-modified HA, and aldehyde-modified HA, norbornene-modified HA and aldehyde or hydrazide-modified HA (each time DCC based on hydrazone), S-protected thiolated-HA, thiolated-HA and PEGDA (polyethylene glycol diacrylate), Thiolated-HA and gold nanoparticles, thiolated-HA, thiolated-HA and acrylated-HA, thiol- and hydrazide-modified HA and oxidized alginate, thiolated-HA and thiolated-carboxymethyl cellulose, Pyridyl disulfide-modified HA and macro-crosslinker PEG-dithiol, aldehyde modified-HA and 3,3′-dithiobis (propionic hydrazide) (each time DCC based on disulfide), furan and tyramine-modified HA and maleimide-modified PEG, HA-furan-adipicdihydrazide and HA-furan-aldehyde, furan-modified HA and maleimide-PEG (each time DCC based on Diels Alder), HA modified with benzoxaborole, phenylboronic acid-modified HA and poly (vinyl alcohol), 3-Aminophenylboronic acid-modified HA and a saccharide, boronic acid-modified HA and fructose-modified HA, boronic acid-modified HA and maltose-modified HA (each time SCC based on boronate ester).

One preferred example are BDDE cross-linked HA molecules. Preferred examples for the modification reactions of the —NHCOCH₃ group of a HA molecule include deacetylation, amidation, hemiacetylation, and hemiacetal formation, among others. Amidation methods have been used for deacetylation of the N-acetyl groups of HA that can then react with an acid, and are usually performed using hydrazine sulfate. (see, e.g., J. Tissue Eng, 2017, January-December; 8: 2041731417726464). In a preferred embodiment, the amount of HA is between 0.4% and 5% (such as between 0.4% and 2.5%), more preferred between 0.8% and 5% (such as between 0.8% and 2.5%), (w/w).

The presence of HA can induce the early formation of granulation tissue, inhibit inflammation and promote epithelial turnover and angiogenesis of the connective tissue. The presence of another water-insoluble polymer, e.g., in the form of a carbomer or mannan, enables an even more precise adjustment of the viscosity of a composition than just the presence of hyaluronic acid and enhances the hydrogel formation. The viscosity determines, among other things, the retention time of the gel between the aligner splint and the gum/teeth while wearing the aligner splint. A higher viscosity promotes the residence time, too high a viscosity impairs the distribution of the gel between the aligner splint and the gum/teeth when the aligner splint is put on). Also, the mucoadhesive effect of the HA supports the retention time of active ingredient at the gum surface.

It was observed hydrogel formulation is increased by using at least one further water-insoluble polymer in addition to HA, preferably, the amount of HA is between 0.8% (w/w) and 2.5% (w/w).

Thus, in another preferred embodiment, (b) comprises HA and at least one further water-insoluble polymer, preferably selected from the group consisting of gelatin, (crosslinked) poly(meth)acrylates such as (crosslinked) polyacrylic acids (e.g., carbomers), (crosslinked) polyurethane polymers, (trimethylated) chitosans, alginates, pectin, povidone-iodine, water-insoluble polysaccharides such as mannans, preferably D-galacto-D-mannan, carboxymethyl cellulose, hydroxyethyl cellulose, ethyl cellulose, methyl cellulose, hydroxypropyl cellulose, hydroxypropylmethyl cellulose and Na, K or Li salts of any of the foregoing. D-Galacto-D-mannan, in addition to its ability as a water insoluble polymer is an antioxidant that can protect cell walls and is therefore preferred. All these further water-insoluble polymers are commercially available.

In a further preferred embodiment, (b) comprises, more preferably consists of, HA and at least one other water-insoluble, preferably mucoadhesive, polymer selected from a group consisting of polysaccharides (preferably mannans), carboxymethyl cellulose, hydroxyethyl cellulose, ethyl cellulose, methyl cellulose, hydroxypropyl cellulose (e.g., low substituted hydroxypropyl cellulose CAS 9004-64-2), hydroxypropylmethyl cellulose, (cross-linked) polyacrylic acid, preferably carbomer, and Na, K or Li salts of any of the foregoing (e.g., Na carboxymethyl cellulose).

In one preferred embodiment, (b) comprises, more preferably consists of, AH and at least one further water-insoluble polymer selected from the group consisting of a carbomer, a mannan (preferably D-Galacto-D-mannan), a cellulose (preferably hydroxypropyl cellulose, carboxymethyl cellulose or a Na or K salt of carboxymethyl cellulose), even more preferably consists of AH and at least one further water-insoluble polymer selected from the group consisting of D-Galacto-D-mannan and carbomer.

In a further preferred embodiment, (b) comprises, more preferably consists of, HA and one further water-insoluble polymer from the group consisting of D-Galacto-D-mannan and carbomer, even more preferably D-Galacto-D-mannan, and wherein the amount of the sum of all water-insoluble polymers is between 0.9% (w/w) and 5% (w/w), and wherein the amount of hyaluronic acid is at least 0.8% (w/w).

In one preferred embodiment, the total sum of (b) is between 0.8% (w/w) and 5% (w/w) and the amount of HA is between 0.4% (w/w) and 3% (w/w), more preferred between 0.8% (w/w) and 2.5% such as between 0.8% (w/w) and 2% (w/w) and the amount of at least one further water-insoluble polymer is between 0.4% (w/w) and 2% (w/w). In a more preferred embodiment, the total sum of (b) is between 0.8% (w/w) and 5% (w/w) and the amount of HA is between 0.4% (w/w) and 3% (w/w) and the amount of at least one further water-insoluble polymer, preferably a carbomer, cellulose or mannan, is between 0.03% (w/w) and 2% (w/w).

In a further preferred embodiment, (b) comprises, more preferably consists of, AH and a carbomer, wherein the amount of the carbomer is between 0.03% (w/w) and 2% (w/w), more preferably between 0.3% (w/w) and 1.6% (w/w), or between 0.03% (w/w) and 0.9% (w/w). In a further preferred embodiment, (b) comprises, more preferably consists of, AH and at least one carbomer selected from a group consisting of carbomers with a viscosity between 1000 mPa*s and 45000 mPa*s, more preferably between 4000 mPa*s and 45000 mPa*s such as between 25000 mPa*s and 45000 mPa*s (at 25° C. and 1.013 bar, 0.5%, pH between 7.3 and 7.8), e.g., Carbomer 30,500 to 39,400 (e.g. Carbopol 5984) or Carbomer 35000 (e.g. Carbopol 974P). In a further preferred embodiment, (b) comprises, more preferably consists of, AH and at least one carbomer selected from a group consisting of carbomers with a viscosity of 1000 mPa*s to 45000 mPa*s, such as 25000 mPa*s to 45000 mPa*s, e.g., Carbomer 30,500 to 39,400 (e.g. in Carbopol 5984) or Carbomer 35000 (e.g. Carbopol 974P), wherein the viscosities were determined at defined conditions (concentration 0.5%, pH 7.3-7.8 at 25° C. and 1.013 bar) with a SI Analytics™ Ubbelohde viscometer with ring markings for verification by manual measurements by Fisher scientific, Schwerte, Germany. In a further preferred embodiment, (b) comprises AH and a carbomer, preferably a carbomer with a viscosity between 4000 mPa*s and 45000 mPa*s, such as between 25000 mPa*s and 45000 mPa*s (at 25° C. and 1.013 bar, 0.5%, pH between 7.3 and 7.8), and the amount of the carbomer is between 0.03% (w/w) and 2% (w/w), such as between 0.3% (w/w) and 0.9% (w/w). Preferably, the carbomer has a viscosity between 25000 mPa*s to 45000 mPa*s (in a 0.5% aqueous solution, pH between 7.3 and 7.8, at 25° C. and 1,013 bar, determined with an Ubbelohde viscometer), e.g., Carbomer gel pH 6.5 NRF S43. In a further preferred embodiment, (b) comprises, more preferably consists of, HA (preferably, the amount of HA is between 0.8% (w/w) and 2.5% (w/w)) and a carbomer, preferably a carbomer with a viscosity between 4000 mPa*s and 45000 mPa*s, more preferably between 25000 mPa*s to 45000 mPa*s, and the amount of the carbomer is between 0.03% (w/w) and 2% (w/w), preferably between 0.3% (w/w) and 0.9% (w/w).

In another preferred embodiment, (b) comprises, more preferably consists of HA (preferably, the amount of HA is between 0.8% (w/w) and 2.5% (w/w)) and a mannan. Preferably, the mannan is a D-galacto-D-mannan, more preferably selected from the group consisting of D-galacto-D-mannan from Caesalpinia spinosa gum (CAS 39300-88-4), D-galacto-D-mannan from Ceratonia siliqua (CAS 11078-30-1), and D-galacto-D-mannan polysaccharides (CAS 9000-40-2). However, also D-galacto-D-mannan from other sources such as Adenanthera pavonina, Cyamopsis tetragonolobus, Caesalpinia pulcherrima, Ceratonia siliqua and Sophora japonica or bacteria can be used. In a further preferred embodiment, b) comprises, more preferably consists of, AH (preferably, the amount of HA is between 0.8% (w/w) and 2.5% (w/w)) and a mannan, preferably D-galacto-D-mannan, wherein the amount of the mannan is between 0.5% (w/w) and 2.5% (w/w), more preferably between 0.7% (w/w) and 1.5% (w/w) or between 0.8% (w/w) and 1.3% (w/w).

In a further preferred embodiment, (b) comprises, more preferably consists of, AH (preferably, the amount of HA is between 0.8% (w/w) and 2.5% (w/w)) and a cellulose, preferably selected from the group consisting of carboxymethyl cellulose, carboxymethyl cellulose, hydroxyethyl cellulose, ethyl cellulose, methyl cellulose, hydroxypropyl cellulose, and a Na or K salt, more preferably a Na salt, of any of the foregoing, more preferably selected from hydroxypropyl cellulose and Na carboxymethyl cellulose. In a further preferred embodiment, b) comprises, more preferably consists of, AH (preferably, the amount of HA is between 0.8% (w/w) and 2.5% (w/w)) and a cellulose, preferably hydroxypropyl cellulose, carboxymethyl cellulose or a Na salt of any of the foregoing, wherein the amount of the cellulose is between 0.5% (w/w) and 2.5% (w/w), more preferably between 0.7% (w/w) and 1.5% (w/w) or between 0.7% (w/w) and 1.3% (w/w).

### Polyol (c)

The compositions according to the invention comprises at least one polyol (d) selected from the group consisting of xylitol, erythritol, and sorbitol.

The benefit of said three sugar alcohols in addition to their anticariogenic ability are their functions as humectant and also as sweetener, i.e., said polyols are beneficial to gum health as well as dental health, overall mouthfeel and user acceptability of the composition. Without being bound by the explanation, it can be assumed that said three polyols, cannot be metabolized by the cariogenic bacteria, e.g., of the Streptococcus mutans species, and that said bacteria die as a result. Thereby, they are also prevented from adhering to the tooth surface as plaque bacteria. By preventing plaque, other types of bacteria are also prevented from settling. In one preferred embodiment, (c) comprises, more preferably consists of, at least one polyol selected from the group consisting of xylitol and erythritol, more preferably, wherein the amount of xylitol or erythritol is between 10% (w/w) and 50% (w/w), more preferably between 10% (w/w) and 45% (w/w) such as in the range between 25% (w/w) and 40% (w/w), even more preferably between 15% (w/w) and 35% (w/w) such as in the range between 25% (w/w) and 35%, (w/w) e.g., 30%±2% (w/w).

In a preferred embodiment, (d) consists of xylitol, preferably, the amount of xylitol is between 10% (w/w) and 50% (w/w), more preferably between 10% (w/w) and 45% (w/w) such as in the range between 25% (w/w) and 40% (w/w), even more preferably between 15% (w/w) and 35% (w/w) such as in the range between 25% (w/w) and 35%, (w/w) e.g., 30%±2% (w/w).

In another preferred embodiment, (d) consists of sorbitol, preferably, the amount of sorbitol is between 10% (w/w) and 50% (w/w), more preferably between 15% (w/w) and 50% (w/w), even more preferably between 15% (w/w) and 45% (w/w), such as in the range between 25% (w/w) and 40% (w/w) or in the range between 25% (w/w) and 35% (w/w), e.g., 30%±2% (w/w).

In another preferred embodiment, (d) consists of erythritol, preferably, the amount of erythritol is between 10% (w/w) and 50% (w/w), more preferably between 15% (w/w) and 50% (w/w), even more preferably between 15% and 45% (w/w), such as in the range between 25% and 40% or in the range between 25% (w/w) and 35% (w/w), e.g., 30%±2% (w/w).

In another preferred embodiment, combinations of two or three of said three polyols can be present as long as the sum of these combinations is at least 10% (w/w) and at most 50% (w/w).

### Anti-Inflammatory Component (d)

A composition in accordance with the invention further comprises at least one other anti-inflammatory component (d), in addition to (b) and (c), selected from a group consisting of selected from the group consisting of alpha-bisabolol, chamazulene, matricin, eugenol, curcumin, camphor, cineole, thujone, ursolic acid, beta-caryophyllene, chlorhexidine, povidone iodine, cetylpyridinium chloride, and cetylpyridinium bromide, more preferably selected from the group consisting of alpha-bisabolol, chamazulene, matricin, eugenol, curcumin, beta-caryophyllene, chlorhexidine, povidone iodine, and cetylpyridinium chloride or bromide, even more preferably selected from a group consisting of alpha-bisabolol, chamazulene, matricin, curcumin, beta-caryophyllene, chlorhexidine, and cetylpyridinium chloride. Preferably, the amount of the sum of all others anti-inflammatory components (d) is between 0.001% (w/w) and 2% (w/w), preferably between 0.001% (w/w) and 1.5% (w/w) between 0.001% (w/w) and 0.5% (w/w). An anti-inflammatory component (d) can be added in isolated form to a composition (e.g., purchased from Merck) or can be added in some cases, e.g., in form of a plant oil or extract. In the latter case, information regarding the content of said anti-inflammatory component (d) in an oil or extract can be received from the producer or the skilled person can determine the amount with methods known in the art, e.g., using GC/FID and the commercially available standards for each of the components as internal reference.

Preferably, (d) consists of, one or more compound(s) selected from the group consisting of alpha-bisabolol, chamazulene, matricin (natural sources in each case e.g. chamomile), eugenol (natural source e.g. clove), curcumin (natural source e.g. turmeric), beta-caryophyllene (natural source e.g. sage or Eucalyptus globulus), camphor, cineole and thujone (natural source e.g. sage), and ursolic acid (natural source e.g. sage); more preferably selected from a group consisting of alpha-bisabolol, chamazulene, matricin, eugenol, beta-caryophyllene, curcumin; even more preferably selected from a group consisting of alpha-bisabolol, chamazulene, matricin, curcumin, beta-caryophyllene. In another preferred embodiment, (d) consists of one component selected from the group consisting of chlorhexidin and cetylpyridinium chloride or cetylpyridinium bromide, more preferably chlorhexidin and cetylpyridinium chloride

In one preferred embodiment, (d) comprises, more preferably consists of, eugenol. In another more preferred embodiment, (d) comprises alpha-bisabolol. In yet another more preferred embodiment, (d) comprises chamazulene. In yet another more preferred embodiment, (d) comprises matricin. In yet another more preferred embodiment, (d) consists of alpha-bisabolol, chamazulene, matricin, or a combination of two or three of the foregoing. In yet another more preferred embodiment, (d) comprises, more preferably consists of, curcumin. In yet another more preferred embodiment, (d) comprises, more preferably consists of, beta-caryophyllene. In yet another more preferred embodiment, (d) comprises, more preferably consists of, chlorhexidine. In yet another more preferred embodiment, (d) comprises, more preferably consists of, cetylpyridinium chloride. In yet another more preferred embodiment, (d) comprises, more preferably consists of, cetylpyridinium bromide.

In yet another more preferred embodiment, (d) consists of one or more components selected from the group consisting of alpha-bisabolol, chamazulene, matricin, a combination of two or three of the foregoing, curcumin, and beta-caryophyllene, preferably, wherein the amount of (d) is between 0.001% (w/w) and 0.06% (w/w), more preferably, between 0.005% (w/w) and 0.04% (w/w), even more preferably between 0.005% (w/w) and 0.02% (w/w). In yet another preferred embodiment, (d) consists of chlorhexidine, preferably, wherein the amount of (d) is between 0.001% (w/w) and 0.1% (w/w), more preferably between 0.001% (w/w) and 0.06% (w/w), even more preferably between 0.005% (w/w) and 0.04% (w/w) or even more preferably between 0.005% (w/w) and 0.02% (w/w). In yet another preferred embodiment, (d) consists of cetylpyridinium chloride, preferably, wherein the amount of (d) is between 0.001% (w/w) and 0.1% (w/w), more preferably between 0.001% (w/w) and 0.06% (w/w), even more preferably between 0.005% (w/w) and 0.04% (w/w) or even more preferably between 0.005% (w/w) and 0.02% (w/w). In yet another preferred embodiment, (d) consists of cetylpyridinium bromide, preferably, wherein the amount of (d) is between 0.001% (w/w) and 0.1% (w/w), more preferably between 0.001% (w/w) and 0.06% (w/w), even more preferably between 0.005% (w/w) and 0.04% (w/w) or even more preferably between 0.005% (w/w) and 0.02% (w/w).

In another preferred embodiment, e.g., if a composition is provided over a longer time period or a user has no severe inflammatory, a composition is free (i.e., does not comprise) a component selected from the group consisting of chlorhexidine, cetylpyridinium chloride, cetylpyridinium bromide, and povidone iodine.

In a further preferred embodiment, a component (d) is provided in the form of a vegetable oil or vegetable extract added to a composition according to the invention. Oil from a plant and extract from a plant are preferably selected from a group consisting of chamomile (Matricaria chamomilla), clove (Syzygium aromaticum), rosemary (Salvia rosmarinus), turmeric (Curcuma longa), and sage (Salvia officinalis), more preferably selected from a group consisting of chamomile (Matricaria chamomilla), clove (Syzygium aromaticum), rosemary (Salvia rosmarinus), turmeric (Curcuma longa), and sage (Salvia officinalis), even more preferably selected from the group consisting of chamomile (Matricaria chamomilla) and turmeric (Curcuma longa).

Important components in chamomile (preferably flowers) is, among other things, matricin (prochamazulene, (−)-(3S,3aR,4S,9R,9aS,9bS)-9-hydroxy-3,6,9-trimethyl-2-oxo-3,3a,4,5,9a, 9b-hexahydroazuleno-[4,5-b]furan-4-yl acetate), chamazulene (7-ethyl-1,4-dimethylazulene) and alpha-bisabolol ((2S)-6-methyl-2-[(1S)-4-methylcyclohex-3-en-1-yl]hept-5-en-2-ol) (anti-inflammatory, (mucous)skin soothing). Chamazulen, a secondary product of Matricin (by splitting off acetic acid and water, both substances are anti-inflammatory) is readily soluble in non-polar solvents and lower alcohols such as ethanol or 1,2-propanediol; alpha-bisabolol is readily soluble in ethanol and insoluble in water, it makes up to 45% of chamomile essential oils. Under certain extraction conditions, matricin is converted to chamazulene.

In a preferred embodiment, a component (d) is therefore provided in form of an extract, preferably an alcoholic extract of chamomile, preferably of chamomile flowers, with the extraction medium comprising (aqueous) alkanols having one to four carbon atoms, preferably ethanol or 1,2-propanediol, wherein the alkanol amount in the extraction medium is between 40% (w/w) and 100% (w/w) based on the weight of the extraction medium. An extraction of chamomile flowers is usually carried out at a temperature range of 5° C. to 60° C. Extracts usually contain at least four parts by weight of extract, preferably four to eight parts by weight, in particular five to six parts by weight of extract, per part by weight of dry weight of fresh chamomile (determined by drying a sample at 105° C. in a drying cabinet for three hours). Chamomile extracts, approved in particular for the cosmetic and medical fields, are commercially available, e.g., Kamillosan (PZN: 00565073). Provider can provide information regarding the content of alpha-bisabolol, matricin and/or chamazulene in an extract. In a further preferred embodiment, a component (d) is provided in form of a CO₂ extract from chamomile, preferably from chamomile flowers. Such methods are well known in the art, e.g., from EP 0058365. Equipment for such extractions (e.g., from Separeco) and CO₂ extracts (e.g., from Flavex, DE-Oko-013 product no. 019.002) are commercially available. Provider can provide information regarding the content of alpha-bisabolol, matricin and/or chamazulene in an extract.

In a further preferred embodiment, a composition according to the invention comprises alpha-bisabolol as a component (d). Preferably the amount of alpha-bisabolol is at least 0.001% (w/w), such as at least 0.003% (w/w), e.g., between 0.003% (w/w) and 1.5% (w/w) such as between 0.005% (w/w) and 1.2% (w/w) or between 0.005% (w/w) and 1.5% (w/w), such as between 0.007% (w/w) and 1.1% (w/w) or between 0.007% (w/w) and 1.5% (w/w), or such as between 0.003% (w/w) and 0.008% (w/w) or between 0.003% (w/w) and 1.5% (w/w). The skilled person is aware how to determine the amount of alpha-bisabolol in a composition with methods known in the art, e.g. via GC/MS and GC/FID using a GC equipment (Shimadzu, model 17A) connected to a flame ionization detector (GC-FID), equipped with a fused silica capillary column with a fused silica capillary column ((SBP5-Supelco, 30 m×0.25 mm i.d., 0.25 μm film thickness). A dichloromethane solution of alpha-bisabolol (9.6 mg/ml) can be prepared for the GC External Standard (GC-ES), and GC Internal Standard (GC-IS). The column temperature can be programmed to vary from 40° C. (4 min) to 200° C. at a rate of 3° C./min. The injector and the detector can be kept at 200° C. and 240° C., respectively. Carrier gas N₂, flow rate 1.80 ml/min. An amount of 1.0 μl (1.0% w/v solution of the composition in dichloromethane) can be injected, split ratio 1:10. The content of alpha-bisabolol in the composition can be calculated from the percentage of its peak area in relation to the total area of the chromatogram (see, e.g., Cerceau et al in Talanta, Vol. 162, Dec. 1, 2016, p. 71-79) or using GC/FID and a commercially available standard of alpha-bisabolol (e.g. from Merck) for preparing a calibration curve and determination of the retention time. Alpha-bisabolol, CO₂ extracts, ethanolic extracts of chamomile as well as other (naturally occurring) sources of alpha-bisabolol can be purchased commercially.

In a further preferred embodiment, a composition according to the invention comprises chamazulene as a component (d). Preferably the amount of chamazulene is at least 0.0006% (w/w), such as at least 0.002% (w/w), e.g., between 0.002% (w/w) and 1% (w/w) such as between 0.002% (w/w) and 0.8% (w/w), such as between 0.003% (w/w) and 0.8% (w/w), such as between 0.003% (w/w) and 0.5% (w/w), such as between 0.004% (w/w) and 0.04% (w/w). The skilled person is aware how to determine the amount of chamazulene in a composition with methods known in the art, e.g., via GC-MS and GC/FID using as internal standard n-hexadecane using an Agilent 7890 A gas chromatograph, an Agilent 5975 C mass spectrometry equipped with a fused silica capillary HP-5 column (30 m length×0.25 mm i.d., 0.25 μm film thickness), carrier gas He, flow rate 1.1 ml/min. The oven temperature program starts at 70° C. (hold for 1 min), then the column can be sequentially heated at a rate of 10° C./min to 155° C. Thereafter, the column can be heated at a rate of 4° C./min to 210° C. and can be hold for 1 min. Then, the column can be heated at a rate of 8° C./min to 270° C. and hold for 2 min. split ratio 1:50 with ionization voltage of 70 eV (for MS). Both the transfer line temperature and the injector temperature can be programmed at 280° C. and 250° C., respectively (see, e.g., Ghasemi et al in Foods 2016, 5, 56:); or using GC/FID and a commercially available standard of chamazulene (e.g., from Merck/Sigma Aldrich) for preparing a calibration curve and determination of the retention time. Chamazulene and ethanolic extracts of chamomile as well as other (naturally occurring) sources of chamazulene can be purchased commercially

In a further preferred embodiment, a composition according to the invention comprises matricin as a component (d). Preferably the amount of matricin is at least 0.0003% (w/w), such as at least 0.001% (w/w), e.g., between 0.001% (w/w) and 0.5% (w/w) such as between 0.001% (w/w) and 0.4% (w/w), such as between 0.002% (w/w) and 0.02% (w/w). The skilled person is aware how to determine the amount of matricin in a composition with methods known in the art, e.g., via GC/MS and GC/FID using an Agilent Technologies 7890B gas chromatograph, equipped with a silica capillary column, HP-5MS (5% diphenyl- and 95% dimethyl-polysiloxane, 30 m×0.25 mm, 0.25 μm film thickness; Agilent Technologies, USA) and coupled with inert, selective 5977 A mass detector of the same company. 3 μl sample dissolved in diethyl ether in a concentration of 1 ppm can be injected in 5:1 split mode, carrier gas He, flow rate 1 ml/min. The oven temperature can be programmed from 50° C. for 2.25 minutes and then increased to 290° C. at the rate of 4° C./min. Temperatures of the MSD transfer line, ion source and quadruple mass analyzer can be set at 300° C., 230° C. and 150° C., respectively. The ionization voltage can be 70 eV and mass range m/z 35-650 (for MS). GC/FID analysis can be carried out under identical experimental conditions as GC/MS. The temperature of the flame-ionization detector (FID) can be set at 300° C. Data processing performed using MSD Chem-Station, MassHunter Qualitative Analysis and AMDIS_32 software (Agilent Technologies, USA). Retention indices of the components from the analyzed samples can be experimentally determined using a homologous series of n-alkanes from C8-C20 as standards. (see, e.g., Stanojevic et al in Ramadan et al in Journal of Essential Oil Bearing Plants, 19:8, 2017-2028, DOI: 10.1080/0972060X.2016.1224689) or using GC/FID and a commercially available standard of matricin (e.g., from Sigma Aldrich) for preparing a calibration curve and determination of the retention time. Matricin, CO₂ extracts of chamomile as well as other (naturally occurring) sources of matricin can be purchased commercially.

In a further preferred embodiment, a composition according to the invention comprises curcumin as a component (d). Preferably the amount of curcumin (a component (d) with colorant properties) is at least 0.001% (w/w), such as at least 0.003% (w/w), e.g., between 0.003% (w/w) and 1.5% (w/w) such as between 0.005% (w/w) and 1.2% (w/w), such as between 0.007% (w/w) and 1.1% (w/w), or such as between 0.003% (w/w) and 0.008% (w/w). The skilled person is aware how to determine the amount of curcumin in a composition with methods known in the art, e.g., via NMR and LC-mass tandem spectrometry using curcumin (HPLC grade, Merck) in DMSO-d6 for producing a calibration curve, 1,3,5-trimethoxy benzene can be used as an internal reference. For NMR, a Varian 600 MHz spectrometer with an ID-6508 indirect probe (S/N=1084) can be used: number of scans 64; relaxation delay 1.0 s; and pulse degree 45°. Peak height can be calculated for the quantitative analysis (see, e.g., Goeren et al in Food Chemistry 113 (2009) 1239-1242). Curcumin is an important component of turmeric (Curcuma longa) oil. Curcumin is good soluble in non-polar solvents. It can be extracted from turmeric using methanol, ethanol, acetonitrile, dimethyl sulfoxide, acetone, and other polar solvents, as well as using hexane and cyclohexane. In a preferred embodiment, curcumin is part of an oil extract of turmeric. For example, at least four parts by weight of extract (preferably a vegetable oil such as rapeseed oil, olive oil or sunflower oil), preferably four to eight parts by weight, in particular five to six parts by weight of extract, per part by weight is used per part of weight of turmeric (determined by drying a sample at 105° C. in a drying cabinet for three hours). Curcumin, turmeric oil, dried and ground turmeric as well as other (naturally occurring) sources of curcumin can be purchased commercially.

In a further preferred embodiment, a composition according to the invention comprises beta-caryophyllene as a component (d). Preferably the amount of beta-caryophyllene is at least 0.001% (w/w), such as at least 0.003% (w/w), e.g., between 0.003% (w/w) and 1.5% (w/w) such as between 0.005% (w/w) and 1.2% (w/w), such as between 0.007% (w/w) and 1.2% (w/w) or between 0.007% (w/w) and 0.1% (w/w). Beta-caryophyllene is a component of clove (Syzygium aromaticum) and Eucalyptus globulus oil. The skilled person is aware how to determine the amount of beta-caryophyllene in a composition with methods known in the art, e.g., via GC/MS and GC/FID using an Agilent 6890 chromatograph coupled to an Agilent 5973N selective mass detector, operated at 250° C. using a HP-5MS capillary column (5%-phenyl-95%-dimethylpolysiloxane, 30 m×0.25 mm×0.25 μm) for separation. For quantification, an Agilent 7890A chromatograph equipped with a flame ionization detector (FID), operated at 280° C. can be used and the electronic integration of the FID signal dividing the area of each component by the total area can be used to quantify beta caryophyllene (see, e.g., Santos et al in Phytomedicine Plus 1 (2021) 100100) or using GC/FID and a commercially available standard of beta caryophyllene (e.g. from Sigma Aldrich) for preparing a calibration curve and determination of the retention time. In a preferred embodiment, beta-caryophyllene is part of a clove oil extract (between 5% and 12% or rosemary oil extract. At least four parts by weight of extract (preferably a vegetable oil such as rapeseed oil, olive oil or sunflower oil), preferably four to eight parts by weight, in particular five to six parts by weight, of extract is used per part by weight of dry weight of cloves or rosemary (determined by drying a sample at 105° C. in a drying cabinet for three hours). Beta-caryophyllene (e.g., from TCI Chemicals), clove or rosemary oil as well as dried and possibly ground cloves or rosemary as well as other (naturally occurring) sources of beta-caryophyllene can be purchased commercially. In a further preferred embodiment, the amount of an oil extract from cloves or Eucalyptus globulus is in a range between 0.1% (w/w) and 5% (w/w), more preferably between 0.1% (w/w) and 3% (w/w), even more preferably between 0.1% (w/w) and 1% (w/w), e.g. between 0.15% (w/w) and 0.7% (w/w).

In a further preferred embodiment, a composition according to the invention comprises ursolic acid as a component (d). Preferably the amount of ursolic acid is at least 0.001% (w/w), such as at least 0.003% (w/w), e.g., between 0.003% (w/w) and 1.5% (w/w) such as between 0.005% (w/w) and 1.2% (w/w), such as between 0.007% (w/w) and 0.1% (w/w). Ursolic acid, sage oil, fresh or dried sage, as well as other (naturally occurring) sources (e.g., Malus domestica, Salvia triloba, Thymus vulgaris, Thymus serpyllum, Lavandula latifolio, Rosmarinus officinalis) of ursolic acid can be purchased commercially. The skilled person is aware how to determine the amount of ursolic acid in a composition with methods known in the art, e.g., via HPLC using a Shimadzu LC-1 OAD pump system equipped with a Shimadzu SPD-MI0A photodiode array detector with the detection wavelength set at 206 nm, a reversed-phase column (Shimpack CLC-ODS (M) 4.6 mm×15 cm particle size 5 μm). Elution rate 0.5 ml/min A:B solvent system: A-acetonitrile; B-1.25% H₃PO₄ aqueous (A:B=86:14 (v/v), injection volume 20 μl (using ursolic acid (e.g., from Sigma Aldrich) for preparing a calibration curve) (see, e.g., Silva et al in Molecules 2008, 13, 2482-2487; DOI: 10.3390/molecules13102482). Ursolic acid, sage oil as well as dried and possibly ground sage as well as other (naturally occurring) sources of ursolic acid can be purchased commercially. In a preferred embodiment, ursolic acid is part of a sage oil extract. At least four parts by weight of extract (preferably with a vegetable oil such as rapeseed oil, olive oil or sunflower oil), preferably four to eight parts by weight, in particular five to six parts by weight of extract, is used per part by weight of dry weight of sage (determined by drying a sample at 105° C. in a drying cabinet for three hours). In another preferred embodiment, the amount of a sage oil extract is in a range between 0.1% (w/w) and 5% (w/w), more preferably between 0.1% (w/w) and 3% (w/w), even more preferably between 0.1% (w/w) and 1% (w/w), e.g., between 0.15% (w/w) and 0.7% (w/w).

In a further preferred embodiment, a composition according to the invention comprises eugenol (CAS 97-53-0) as a component (d). Preferably the amount of eugenol is at least 0.001% (w/w), such as at least 0.003% (w/w), e.g., between 0.003% (w/w) and 1.5% (w/w) such as between 0.005% (w/w) and 1.2% (w/w), such as between 0.007% (w/w) and 0.1% (w/w). Clove oil comprises between 65% to 85% eugenol. In one preferred embodiment, a composition comprises clove oil, preferably, wherein the amount of clove oil in a composition is between 0.0015% (w/w) and 1.76% (w/w). The skilled person is aware how to determine the amount of eugenol in a composition with methods known in the art, e.g., using a Shimadzu GC-2010 gas chromatograph equipped with an FID and a DB-5 fused-silica column (30 m×0.25 mm i.d., film thickness 0.25 μm, Agilent). Oven temperature 80° C. for 2 min, then heating from 80 to 230° C. at a rate of 6° C./min, and at 230° C. for 2 min. Injector and detector temperatures can be 230° C., carrier gas N₂, flow rate 24 ml/min. The samples (0.1% in absolute ethanol) can be injected into the GC with a split ratio of 1:20. Methyl salicylate can be used as internal standard (see, e.g., Guan et al in Food Chemistry 101 (2007) 1558-1564) or using GC/FID and a commercially available standard of eugenol (e.g. from Merck) for preparing a calibration curve and determination of the retention time.

In a further preferred embodiment, a composition according to the invention comprises camphor (CAS 76-22-2) as a component (d). Preferably the amount of camphor is at least 0.001% (w/w), such as at least 0.003% (w/w), e.g., between 0.003% (w/w) and 1.5% (w/w) such as between 0.005% (w/w) and 1.2S w/w), such as between 0.007% (w/w) and 0.1% (w/w).

The skilled person is aware how to determine the amount of camphor in a composition with methods known in the art, e.g., via GC/MS and GC/FID with cyclodecanone as internal reference using an Agilent model 6890 Series Plus gas chromatograph in combination with a CTC Combi PAL autosampler and an Agilent 5973N mass selective detector or FID detector using a fused silica capillary column (HP-Innowax, 60 m×0.25 mm i.d., film thickness 0.25 μm). Temperature program: 45° C. hold for 1 min, 5° C./min up to 180° C., 25° C./min up to 240° C., hold for 5 min. The temperatures for the injection port, ion source, quadrupole and interface can be set at 240° C., 230° C., 150° C., and 250° C., respectively. Split/splitless injection port (1 μl, splitless), carrier gas He, flow rate 1.0 ml/min. Data acquisition and analysis can be performed using standard software supplied by the manufacturer. For quantification, peak area ratios of the analytes to the internal standard cyclodecanone can be calculated as a function of the concentration of the substances (see, e.g., Walch et al. in Chemistry Central Journal 2011, 5:44); or using GC/FID and a commercially available standard of camphor (e.g. from Sigma Aldrich) for preparing a calibration curve and determination of the retention time.

In a further preferred embodiment, a composition according to the invention comprises cineole (CAS 470-82-6) as a component (d). Preferably the amount of cineole is at least 0.001%% (w/w), such as at least 0.003%% (w/w), e.g., between 0.003%% (w/w) and 1.5%% (w/w) such as between 0.005%% (w/w) and 1.2%% (w/w), such as between 0.007%% (w/w) and 0.1%% (w/w). The skilled person is aware how to determine the amount of cineole in a composition with methods known in the art, e.g., via GC using an Agilent 7890B/7000D GC-MS/MS instrument. 1 μl samples can be injected split ratio 1:5 onto an HP-5 ms fused silica capillary column (30 m, 0.25 mm i.d., 0.25 μm film thickness) with He as carrier gas, flow rate 2.25 ml/min. The inlet temperature can be set at 250° C. Program: the initial temperature of 100° C. (held for 1 min) can be raised to 280° C. at the rate of 25° C./min and can be held constant for 1.5 min. The total run time can be 23.7 min. The MS conditions were: electron impact ionization (EI) mode; transfer line temperature, 280° C.; ionization energy, 70 eV; ion source temperature, 230° C.; MS quadrupoles temperature, 150° C. with weighed cineol in n-hexane for preparing a calibration curve (see, e.g., Sa et al. in Biomedical Chromatography 2021; 35:e5080.) or using GC/FID and a commercially available standard of cineole (e.g. from Merck) for preparing a calibration curve and determination of the retention time. Cineole is the major component of Eucalyptus (between 70% and 90% of eucalyptus oil).

In a further preferred embodiment, a composition according to the invention comprises thujone as a component (d). Thujone appears predominantly in two diastereomeric forms, (−)-α- and (+)-β-thujone. Preferably the amount of thujone is at least 0.001% (w/w), such as at least 0.003% (w/w), e.g., between 0.003% (w/w) and 1.5% (w/w) such as between 0.005% (w/w) and 1.2% (w/w), such as between 0.007% (w/w) and 0.1% (w/w). The skilled person is aware how to determine the amount of thujone in a composition with methods known in the art, (see, e.g., Walch et al. in Chemistry Central Journal 2011, 5:44 above) or using GC/FID and a commercially available standard of thujone (e.g., from Sigma Aldrich) for preparing a calibration curve and determination of the retention time.

In a further preferred embodiment, a composition according to the invention comprises cuminaldehyde (CAS 122-03-2) as a component (d). Preferably the amount of cuminaldehyde is at least 0.001% (w/w), such as at least 0.003% (w/w), e.g., between 0.003% (w/w) and 1.5% (w/w) such as between 0.005% (w/w) and 1.2% (w/w), such as between 0.007% (w/w) and 0.1% (w/w). The skilled person is aware how to determine the amount of cuminaldehyde in a composition with methods known in the art, e.g., using an Agilent GC 7890A gas chromatograph with an FID and a HP-5 capillary column (30 m×0.25 mm, 0.25 μm film thicknesses). The initial oven temperature can be held at 50° C. for 3 min, increased up to 120° C. with a heating rate of 3° C./min; then the column temperature can be programmed as 120° C. to 250° C. by a heating rate of 5° C./min and held at this temperature for 5 min, carrier gas N₂, flow rate 2 ml/min. The injector temperature and detector temperature can be adjusted to 280° C. and 300° C., respectively. Sample size 1.0 μl with a split ratio of 1:10 (see, e.g., Bahman et al in J Oleo Science, 63, (7), 741-746 (2014). Cuminaldehyde (e.g., Cuminaldehyde≥98%, FCC, FG from Merck) can be used for preparing a calibration curve.

In one preferred embodiment, a composition according to the invention comprises as a component (d) at least one compound selected from the group of alpha-bisabolol, matricin, and chamazulene. In another preferred embodiment, a composition according to the invention comprises as a component (d) at least one compound selected from the group of alpha-bisabolol, matricin, and chamazulene and further ingredients of a chamomile extract or oil. In another embodiment, a composition according to the invention comprises as a component (d) curcumin. In another preferred embodiment, a composition according to the invention comprises as a component (d) curcumin and further ingredients of an oil extract of turmeric.

The anti-inflammatory component (d) is in one embodiment added in form of an oil or extract from chamomile (Matricaria chamomilla), clove (Syzygium aromaticum), rosemary (Salvia rosmarinus), Eucalyptus globulus, turmeric (Curcuma longa) and sage (Salvia officinalis), particularly preferably from chamomile (Matricaria chamomilla).

It is clear to those skilled in the art that the concentrations of the ingredients in herbal essential oils or extracts can vary depending on the geographic origin of the plants, the variety of the plants and the processing of the plant parts, but these substances are present in sufficient quantity to achieve the desired effect in oils from these plants or an extraction with non-polar solvents or an extraction with alcohol and/or water.

### Further Component(s) (in One Preferred Embodiment, Further Components (e))

In addition to the essential components (a) to (d), the oral care composition may contain further additives, e.g. a source of vasodilation components, antioxidants, emulsifiers, etc. If products of further components (e) for the preparation of a composition according to the invention comprise components of groups (a) to (d), these amounts in such products are assigned to the corresponding groups (a) to (d) when calculating the various amounts. For example, propolis can be one further component (e) (see e1). However, if a propolis extract product also contains water or ethanol (e.g., an ethanolic extract of propolis), these amounts of water or ethanol are subtracted from the amount of used propolis product and assigned to group (a) (see, e.g., example 1).

In some cases, further components can be assigned to several sub-categories of (e). For example, salts such as stannous fluoride comprise a metal ion (e22) and a fluoride anion (e14). However, this is irrelevant, since in (e) only the total amount of other additives is relevant (i.e., the amount of stannous fluoride is counted); or a compound (e) can be mentioned in more than one sub-group (e) but is only counted once for evaluating the amount of compounds (e) in a composition, e.g., lactic acid is a hydroxy acid (e16) but also has humectant effects (e7) but is only counted once when evaluating the amount of components (e).

For clarification's sake, if a component of group (a) to (d) has further properties that could be assigned to “further components (e)” in addition to an essential property as required for a) to d), the amount of the essential component only counts towards the amount of the essential components (a) to (d) in the overall composition. For example, propylene glycol is a protic solvent. However, it also has humectant and preservative effects. In a composition according to the invention, the level of propylene glycol (if present) is attributed to the protic solvents (a), but not the level of “further components” (e) which may also include other preservatives and humectants.

Further components (such as (e)) are either components which come along with compounds (a) to (d), e.g., a carbomer product such as Carbomer gel pH 6.5 NRF S43 comprises next to Carbomer 35.000, further additives, namely trometamol (Tris(hydroxymethyl)aminomethane (TRIS)) (buffer), Na edetate (Na ethylenediaminetetraacetate (EDTA)) (complexing agent), propylene glycol (protic solvent) and water (protic solvents); or further components (such as (e)) are added to a composition in accordance with the present invention. Thus, the skilled person will understand that further components (such as (e)) encompasses any component which comes along as auxiliary, solvent or other addition in a product commercially available at the filing date of this application which is used to provide a compound of group (a), (b), (c), (d) or optionally (e) to a composition according to the invention.

The skilled person understands that up to 10% of a composition according to the invention can represent compounds which are not compounds of group (a) to (d) as defined herein. Thus, the skilled person will understand that a limiting list of such additives is not required but additives at the filing date of this application can be added to an oral care composition of the invention, as long as a combination of such further components (e) does not lead to a formulation which would no longer fall under into the scope of the present invention. The skilled person will understand, an addition of up to 10% of further components (such as (e)) can positively enhance aspects of a composition in accordance with the invention, e.g., the good flow behavior of a gel according to the invention can be further improved, or addition of such components can improve the acceptance of a composition by a user (e.g. better taste, different color, better sensory feeling of a composition), or even further supply of beneficial components for the gum such as vitamins or oral cavity fortifying polyols (e.g., dexpanthenol) can improve the good effects of an already novel and inventive composition in accordance with the present invention.

Thus, in one preferred embodiment, a composition according to the invention comprises one or more further component(s) (preferably (e)), wherein the amount of the sum of said further components (preferably (e)) is 10% (w/w) or less. In yet another preferred embodiment, a composition comprises as a further component (preferably (e)) at least one antioxidant, preferably one or more catechins, wherein the amount of the sum of all further components (e) is 10% (w/w) or less. Non-limiting but preferred examples for further components are those compounds listed in sub-groups (e1) to (e22). In yet another preferred embodiment, a composition comprises one or more further component(s) (e) selected from the group consisting of (e1), (e2), (e3), (e4), (e5), (e7), (e8), (e9), (e12), (e14), (e15), (e16), (e18), (e21), and (e22). In yet another preferred embodiment, (e) in a composition consists of one or more compounds selected from the group consisting of (e1), (e2), (e3), (e4), (e5), (e7), (e8), (e9) (e12), (e14), (e15), (e16), (e18), (e21), and (e22). In a more preferred embodiment, a composition comprises at least one compound of group (e4), even more preferably at least one catechin selected from the group consisting of C, EC, ECG, EGC and EGCG. Preferred components (e1) are lotus extract, propolis or propolis extract, chamomile oil or extract, clove oil or extract, Mentha piperita oil or extract, Mentha arvensis oil or extract turmeric oil or extract, sage oil or extract, Eucalyptus globulus oil or extract, Mentha spicata oil or extract, Micromeria fruticosa oil or extract, Artemisia dracunculus oil or extract, Origanum vulgare oil or extract, Rosmarinus officinalis oil or extract, Thymus oil or extract, Ocimum basilicum oil or extract, Origanum majorana oil or extract, Theobroma cacao extract, Camellia sinensis extract, in one preferred embodiment, extracts are provided in form of a lyophilizate.

In one preferred embodiment, a composition comprise one or more (e1) aprotic solvents such as oils and extracts of plants and animal-originated extracts, e.g., propolis or propolis extract (propolis is produced by bees and has antibiotic, viral and fungal properties. Propolis is a mixture of many different substances. It consists of about 55% natural resin and pollen balm, about 30% wax, about 5% pollen, about 10% essential oils from the flower buds and saliva secretion. Resign and pollen balm of propolis are rich in flavonoids, more preferably one or more flavonoid(s) selected from the group consisting of chrysin, galangin, pinocembrin, pinobanksin acetate, prenylflavonoid, isonymphaeol-B, nymphaeol-A, nymphaeol-B and nymphaeol-C. Propolis further comprises next to flavonoids, rubber, phenols (cinnamic acid, coumaric acid, caffeic acid, ferulic acid, isoferulic acid) and their esters as well as polysaccharides which are not in group (c). Due to the general presence of beneficial oral cavity compounds in propolis, in one preferred embodiment, a composition comprises between 0.005% (w/w) and 2% (w/w) such as between 0.01% (w/w) and 2% (w/w) or between 0.005% (w/w) and 1% (w/w), or between 0.01% (w/w) and 0.8% (w/w) of propolis. Propolis can be used as it is or in form of a pre-dissolved extract. In such a case, it is sufficient to provide the amount of propolis added to a formulation to prepare a composition in accordance with the invention. A quantification of the various components of propolis is not necessary but the amount of propolis added is sued when calculating the total amount of components (e). When a propolis extract is used, of course relevant solvents of group (a) have to be substracted from the amount of propolis extract and added to group (a). Propolis extracts are commercially available (e.g., from Hoyer GmbH).

In a preferred embodiment, a composition comprises a lotus extract more preferably, a composition comprises a lotus extract, wherein the amount of the lotus extract is in a range between 0.1% (w/w) and 5% (w/w), more preferably between 0.4% (w/w) and 2% (w/w). Lotus extracts are rich in vitamins (especially vitamin C in the root and a high amount of vitamin A in general), minerals, especially iron, copper, manganese, zinc and potassium compounds, armepavin, fatty acid, protein, phosphorus and linoleic acid and has a positive effect on the complexion. The high level of potassium in the lotus root dilates the blood vessels, thereby facilitating blood circulation, so lotus extract also has a vasodilation effect. It is preferably an extract from lotus root tissue. It is also preferably an aqueous extract or an extract of water and up to 80% ethanol. In general, like other aqueous and/or alcoholic extracts described herein, a lotus extract can be freeze-dried, and the resulting powder can be used for the preparation of a composition according to the invention. Lotus extracts usually comprise at least four parts by weight extract (preferably four to eight parts by weight, in particular five to six parts by weight) per part by weight dry weight of lotus (determined by drying a sample at 105° C. in a drying cabinet for three hours). Lotus extracts are commercially available in different dosage forms (powder as a freeze-dried extract or liquid as an extract), e.g., from Ruschin.

In regard of the present invention, vegetable oils and their components are regarded as aprotic solvents. A (plant) oil or extract may also comprise traces of specific aprotic solvents selected from the group consisting of acetonitrile, dimethyl sulfoxide, acetone, and other polar solvents, as well as alkanes such as hexane and cyclohexane and halogenated alkanes such as dichloromethane, which may be required for preparing extracts of plants or plat oils. Preferably, the amount of such specific protic solvents in a composition, especially hexane or dichloromethane, is less than 0.002% (w/w).

A vegetable oil comprises between 5% and 80% linolenic acid and does not comprise cholesterol. Most preferably, an oil is liquid at 20° C. (A liquid is a nearly incompressible fluid that conforms to the shape of its container but retains a nearly constant volume independent of pressure). In one preferred embodiment, a composition comprises one or more vegetable oils or extracts, preferably one or more vegetable oils or extracts selected from the group consisting of vegetable oil (if a component of a vegetable oil is a component of group (d), the amount of this component is listed in group (d) and is subtracted from the amount of the vegetable oil being a further component (e)) from chamomile, clove, rosemary, turmeric, sage, oilseed rape (Brassica napus), olive (Olea europaea), sunflower (Helianthus annuus), peppermint (Mentha piperita), lotus (nelumbo), preferably, wherein the amount of the sum of the plant oil(s) is between 0.1% (w/w) and 8% (w/w), more preferably between 0.1% (w/w) and 5% (w/w), even more preferably between 0.1% (w/w) and 3% (w/w), e.g. between 0.15% (w/w) and 0.1% (w/w). Curcumin is an important component in turmeric (Curcuma longa) oil. In a further preferred embodiment, a composition according to the invention comprises turmeric oil. The amount of this oil is preferably in a range between 0.1% (w/w) and 5% (w/w), more preferably between 0.2% (w/w) and 3% (w/w). Beta-caryophyllene can be found in, e.g., clove (Syzygium aromaticum) oil and Eucalyptus globulus oil. In a further preferred embodiment, a composition according to the invention comprises clove oil or Eucalyptus globulus oil. The amount of any of these oils is preferably in a range between 0.1% (w/w) and 5% (w/w), more preferably between 0.2% (w/w) and 3% (w/w). Ursolic acid can be found in, e.g., sage oil (Salvia officinalis). In a further preferred embodiment, a composition according to the invention comprises sage oil. The amount of this oil is preferably in a range between 0.1% (w/w) and 5% (w/w), more preferably between 0.2% (w/w) and 3% (w/w). Eugenol is the major component in clove oil. In a further preferred embodiment, a composition according to the invention comprises sage oil. The amount of this oil is preferably in a range between 0.1% (w/w) and 5% (w/w), more preferably between 0.2% (w/w) and 3% (w/w). Alpha-bisabolol, matricin and chamazulene are important compounds in chamomile extracts/oils. In a further preferred embodiment, a composition comprises an extract of chamomile, preferably a CO₂ extract or ethanolic extract. The amount of this CO₂ or ethanolic extract is preferably in a range between 0.1% (w/w) and 5% (w/w), more preferably between 0.2% (w/w) and 3% (w/w). An important component of various plant oils is menthol ((2-isopropyl-5-methylcyclohexanol). Menthol is inter alia a disinfectant. Menthol is commercially available (e.g., from Roth). It is a major component in mentha oils such as peppermint oil (Mentha piperita), wild mint (Mentha arvensis), or Mentha spicata and other genera and species of the mint family (Labiatae), such as Micromeria fruticosa, tarragon (Artemisia dracunculus), basil (Ocimum basilicum), marjoram (Origanum majorana), oregano (Origanum vulgare), rosemary (Rosmarinus officinalis), sage (Salvia) and thyme (Thymus). In a preferred embodiment, a composition comprises peppermint oil or Mentha arvensis oil. Both can be produced, e.g., by steam distillation of peppermint or wild mint plants, respectively. Menthol, peppermint oils or Mentha arvensis leaf oils as well as the other oils of the mint family are commercially available (e.g., Roth and Essence-pur GmbH). In another preferred embodiment, the amount of peppermint oil, Mentha arvensis leaf oil, Mentha spicata, Micromeria fruticosa, tarragon, basil, marjoram, oregano, rosemary, sage or thyme oil or combinations thereof, more preferably the amount of peppermint oil, Mentha arvensis leaf oil, or Mentha spicata oil in a composition is in a range between 0.001% (w/w) and 5% (w/w), more preferably between 0.1% (w/w) and 3% (w/w), even more preferably between 0.1% (w/w) and 1% (w/w), e.g. between 0.15% (w/w) and 0.7% (w/w). In a further preferred embodiment, a composition comprises Mentha arvensis oil, preferably leaf oil. Mentha arvensis oil, Mentha arvensis extracts as well as oils and extracts (e.g., water steam distillation products) of part of the plant are commercially available. The amount of this oil in a composition is preferably between 0.001% (w/w) and 2.5% (w/w), more preferably between 0.02% (w/w) and 0.5% (w/w). In one preferred embodiment, the amount of menthol in a composition is between 0.0001% and 2% (w/w), more preferably between 0.1% (w/w) to 2% (w/w). If any of these oils is part of a composition, the skilled person can easily calculate the amount of menthol in the oils with the information of the provider. Alternatively, the skilled person can determine the amount of menthol in an oil or a composition using HPLC methods known in the art, e.g., using dilutions of a stock solution of 2.5 mg menthol/5 mg on a chromatographic system of Waters 2695 separation module equipped with an auto injector, Waters 2414 refractive index detector, an inertsil ODS 3V (4.6 mm×250 mm, 5 μm) column using water:methanol (30:70 v/v) as a mobile phase, with an injection volume of 100 μl (see, e.g., Shaikh in J Pharm Bioallied Sci. 2010 October-December; 2(4): 360-364). Group e1 also encompasses all further substances which are present in an oil or extract in addition to the compounds of groups (a) to (d). In one preferred embodiment, a tea extract is part of a composition to provide specific catechins (see e4—antioxidants).

In one preferred embodiment, a composition comprise one or more (e2) buffer compounds (resulting in buffer compound combination) which adjust and/or stabilize the pH value in the range between 6 to 7.5, more preferably in the range between 6.5 and 7.5 and which are usually present in this pH range in form of a weak acid as well as its corresponding base or vice versa, in form of a weak base and its corresponding acid). The skilled person is aware that based on the nature of a compound, the addition of pH reducing or enhancing strong acids or strong bases may be required to adjust the pH of a composition in the presence of a buffer compound (e.g., HCl, NaOH, H₂SO₄, H₃PO₄ etc.). Such pH adjusting compounds are also encompassed in the term buffer. In one preferred embodiment, a composition comprises a buffer selected from the group consisting of TRIS, HEPES (4-(2-hydroxyethyl)-1-piperazineethanesulfonic acid), phosphate-buffers selected from the group consisting of alkali dihydrogen phosphate and alkali hydrogen phosphate comprising buffers, e.g., potassium dihydrogen phosphate or sodium dihydrogen phosphate with optionally various counterparts (e.g., phosphate buffer according to Sorensen (KH₂PO₄/Na₂HPO₄), KH₂PO₄ and NaOH, combinations of alkali chloride salts and alkali dihydrogen and/or dialkali hydrogen phosphate (e.g., PBS: NaCl, Na₂HPO₄ KCl and KH₂PO₄), carbonate buffers (e.g., sodium bicarbonate, potassium bicarbonate, ammonium bicarbonate), citrate buffers (e.g., potassium or sodium citrate and optionally citric acid), preferably, the term alkali in combination with buffer/buffer components refers to alkali ions selected from Na and K, preferably, wherein the amount of the sum of buffer components in a composition is between 0.05% (w/w) and 2% (w/w). In a further preferred embodiment, a composition comprises a buffer selected from the group consisting of TRIS, HEPES, sodium bicarbonate, potassium bicarbonate KH₂PO₄/Na₂HPO₄, sodium citrate (and optionally citric acid) and PBS, even more preferably consisting of TRIS, sodium carbonate and PBS. In a preferred embodiment, a composition comprises TRIS, preferably, wherein the amount of TRIS is between 0.03% (w/w) and 0.9% (w/w), preferably between 0.3% (w/w) and 0.9% (w/w). In another preferred embodiment, a composition comprises sodium bicarbonate, preferably, wherein the amount of sodium bicarbonate is between 0.01% (w/w) and 0.9% (w/w), preferably between 0.01% (w/w) and 0.3% (w/w). In another preferred embodiment, a composition comprises PBS, preferably, wherein the amount of PBS (8,184 g NaCl, 1.8 g Na₂HPO₄ and 0.3 g KH₂PO₄/1000 ml buffer solution) is between 0.01% (w/w) and 0.9% (w/w), preferably between 0.01% (w/w) and 0.3% (w/w).

In one preferred embodiment, a composition comprise one or more (e3) Chelating agents (chemical compound that can form stable complexes with metal ions by coordinating with them through multiple sites). In one preferred embodiment, a composition comprises one or more chelating agent(s) selected from the group consisting of ethylenediaminetetraacetic acid (EDTA), ethylene glycol tetraacetic acid (EGTA), ethylenediamine, citric acid, mercaptodicarboxylic acid, and triethanolamine (TEA), preferably, preferably, wherein the amount of the sum of the said one or more chelating agent(s) in a composition is between 0.001% (w/w) and 5% (w/w), more preferably in a range a range between 0.005% (w/w) and 0.5% (w/w). In one preferred embodiment, a chelating agent is EDTA. In a further preferred embodiment, a composition according to the invention comprises a complexing agent. The amount of the complexing agent is preferably in a range between 0.005% (w/w) and 0.5% (w/w).

In one preferred embodiment, a composition comprise one or more (e4) Antioxidants. In one preferred embodiment, a composition comprises one or more antioxidant(s) selected from the group consisting of L-ascorbic acid or derivatives thereof (e.g. Na ascorbyl phosphate), dibutyl hydroxy toluene, butyl hydroxy anisole, superoxide dismutase, carotenoids, astaxanthin, rutin or derivatives thereof, hesperidin, quercetin, catechin [preferably selected from the group consisting of catechin (C), epicatechin (EC), epigallocatechin (EGC), or derivatives thereof including saccharide derivatives and esters with gallic acid or gallic acid derivatives (preferably ECG or EGCG), more preferably at least one catechin is selected from the group consisting of EC, ECG, EGC and EGCG; gallic acid or derivatives thereof, preferably ethers of a fatty acid and gallic acid, allantoin (N-(2,5-Dioxoimidazolidin-4-yl)urea) and derivatives thereof, preferably aluminium chlorhydroxy allantoinate, aluminium chlorhydroxy allantoinate propylene glycol, aluminium dihydroxy allantoinate, allantoin calcium pantotenate, allantoin glycyrrhetinic acid, allantoin ascorbate, allantoin N-acetyl-DL-methionine, allantoin zinc undecylenate, allantoin ethyl p-aminobenzoate, glutathione or derivatives thereof, glutathione, β-carotenes or derivatives thereof, ubiquinol, polyphenols, sweet hydrangea leaf, turmeric, rose fruit, echinacea, Scutellaria root, Hypericum erectum, Chinese gall nut, Geranium thunbergii, rice, rice bran, comfrey, Xanthoxylum piperitum, labiate, peony root, soybean, “Natto” (soybeans fermented in their own bacteria), tea leaf, clove, loquat, peony, horse chestnut, saxifrage, rooibos, rosemary, spirulina, chlorella and dunaliella, bilirubin, cholesterol, tryptophan, histidine, thiotaurine and hypotaurine, preferably, wherein the amount of the sum of the said one or more antioxidant(s) is between 0.001% (w/w) and 5% (w/w).

In one preferred embodiment, a composition comprises one or more antioxidants selected from the group consisting of ascorbic acid, Na ascorbyl phosphate, catechin, allantoin (or its allantoinate form), and limonene, more preferably selected from the group consisting of EC, ECG, EGC and EGCG, allantoin, allantoinate and limonene.

In one preferred embodiment, a composition comprises allantoin or allantoinate, preferably, wherein the amount of allantoin or allantoinate is between 0.001% (w/w) and 5% (w/w), more preferably between 0.001% (w/w) and 2% (w/w).

In one preferred embodiment, a composition comprises ascorbic acid or Na ascorbyl phosphate, preferably, wherein the amount of ascorbic acid or Na ascorbyl phosphate is between 0.001% (w/w) and 5% (w/w), more preferably between 0.001% (w/w) and 2% (w/w).

It was observed the presence of catechins (without being bound to the explanation, catechins can not only be antioxidant components by activating the glutathionperoxidase but also vasodilation components), the compositions according to the invention showed a further enhancement in gum health and inhibition of plaque formation. Therefore, one preferred embodiment refers to a composition comprising at least one catechin, preferably catechin from green tea or cacao, more preferably at least one catechin selected from the group consisting of EC, ECG, EGC and EGCG; or selected from the group consisting of C and EC.

Determination of catechin (c) and Epicatechin (EC) as well as other catechins such as EGC, ECG and EGCG can be performed using calibration standard solutions of the respective catechin to prepare a calibration curve with HPLC methods known in the art using, e.g., a HPLC system (Shimadzu, 2010 CHT) consisted of quaternary pump with vacuum degasser, thermostated column compartment, autosampler, and UV detector and a reversed phase column (TARGA, C18, 5 μm, 250×4.6 mm) by gradient elution (Solution A: 0.1 ml of orthophosphoric acid dissolved in 900 ml of HPLC grade water and the volume was made up to 1000 ml with water and the solution was filtered through 0.45 μm membrane filter and degassed in a sonicator for 3 minutes, Solution B: acetonitrile. Mobile phase was run using gradient elution: at the time 0.01 min 11% B; at the time 30 min 25% B; at the time 35 to 39 min 100% B; and at the time 40 to 50 min 11% B) with a flow rate of 1.0 ml/min with an operating temperature of 30° C. and detection with a UV-Visible detector at 280 nm (see, e.g., Raju et al in Int. Sch. Res. Notices 2014; 2014; 628196). Catechins for the preparation of calibration curves are available e.g., from Merck, Sigma Aldrich and/or Roth.

In a preferred embodiment, the amount of a catechin component(s) in a composition is between 0.001% (w/w) and 5% (w/w) such as between 0.2% (w/w) and 5% (w/w), more preferably between 0.5% (w/w) and 3.5% (w/w), even more preferably between 1.8% (w/w) and 2.5% (w/w) (e.g. between 2.2% (w/w) and 2.4% (w/w)). Catechins, dried tea, tea extracts, dried cacao and cacao extracts are commercially available. In one preferred embodiment, a composition comprises EGCG; more preferably wherein the amount of EGCG is between 0.2% (w/w) and 5% (w/w), even more preferably between 0.2% (w/w) and 2% (w/w). In another preferred embodiment, a composition comprises EC; more preferably wherein the amount of EC is between 0.03% (w/w) and 0.5% (w/w) such as between 0.03% (w/w) and 0.85% (w/w), even more preferably between 0.03% (w/w) 0.17% (w/w). In yet another preferred embodiment, a composition comprises ECG; more preferably wherein the amount of ECG is between 0.03% (w/w) and 0.85% (w/w) such as between 0.05% (w/w) and 1.5% (w/w), even more preferably between 0.05% (w/w) and 0.3% (w/w). In yet another preferred embodiment, a composition comprises EGC; more preferably wherein the amount of EGC is between 0.03% (w/w) and 0.85% (w/w) such as between 0.09% (w/w) and 2.3% (w/w), even more preferably between 0.09% (w/w) and 0.46% (w/w). In yet another preferred embodiment, a composition comprises a combination of EC, ECG, EGC and EGCG, preferably, wherein the amount of EGCG is between 0.2% (w/w) and 1% (w/w), the amount of EC is between 0.03% (w/w) and 0.17% (w/w), the amount of ECG is between 0.05% (w/w) and 0.3% (w/w), and the amount of EGC is between 0.09% (w/w) and 0.46% (w/w).

In another preferred embodiment, a composition comprises C and EC. In one preferred embodiment, a composition comprises C; more preferably wherein the amount of C is between 0.03% (w/w) and 5% (w/w), even more preferably between 0.05% (w/w) and 1% (w/w). In another preferred embodiment, a composition comprises C and EC, wherein the amount of C is between 0.05% (w/w) and 1% (w/w) and the amount of EC is between 0.05% (w/w) and 4.95% (w/w).

In another preferred embodiment, a composition comprises limonene, more preferably, wherein the amount of limonene is between 0.001% (w/w) and 5% (w/w), more preferably between 0.001% (w/w) and 2% (w/w).

In one preferred embodiment, a composition comprise one or more (e5) Preservatives. In one preferred embodiment, one or more preservatives are selected from the group consisting of benzoic acid and its Na, K, magnesium and calcium salts, in particular the Na salt, propionic acid and its Na, K, magnesium and calcium salts, salicylic acid and its Na, K, magnesium and calcium salts, phenyl methanol, phenoxyethanol, sorbitan caprylate, caprylyl glycol, parabens (e.g., methyl paraben or propyl paraben), preferably, wherein the amount of the sum of one or more preservative(s) is between 0.01% (w/w) and 0.5% (w/w). In one embodiment, a preservative is methylparaben.

In one preferred embodiment, a composition comprise one or more (e6) Humectants (different from compounds of group (a), (b) or (c)). In one preferred embodiment, one or more, more preferably one, humectant(s) is selected from the group consisting of, Aloe vera gel, alpha hydroxy acids (e.g., lactic acid), glyceryl triacetate, LiCl, polymeric polyols (e.g. polydextrose), polyethylene glycol (PEG), Na hexametaphosphate, maltitol, urea, castor oil (vegetable oil pressed from castor beans), preferably, wherein the amount of the sum of the said humectant(s) is between 0.1% (w/w) and 10% (w/w).

In one preferred embodiment, a composition comprise one or more (e7) flavorings. In one preferred embodiment, a composition comprises one or more flavoring(s) selected from the group consisting of wintergreen, cassia, parsley oil, marjoram, lemon, orange, propenylguaethol, heliotropin, 4-cis-Heptenal, diacetyl, methyl-p-tert-butylphenylacetate, menthol, methyl salicylate, 1-ethylone halicylate a-irison, methyl cinnamate, ethyl cinnamate, butyl cinnamate, ethyl butyrate, ethyl acetate, methyl anthranilate, iso-amyl acetate, iso-amyl butyrate, allyl caproate, thymol, cinnamyl alcohol, octanol, octanal, decanol, decanal, phenylethyl alcohol, benzyl teral alcohol, linalool, limonene, citral, neral, geranial, geraniolnerol, maltol, ethylmaltol, anethole, dihydroanethol, carvone, menthone, beta-damascenone, ionone, gamma-decalactone, gamma-nonalactone, and y-undecalactone, preferably, the amount of the sum of one or more flavoring(s) is between 0.001% (w/w) and 5% (w/w), more preferably between 0.01% (w/w) and 4% (w/w).

In one preferred embodiment, a composition comprise one or more (e8) vitamins. In one embodiment, the composition comprises one or more vitamin selected from the group consisting of vitamin A, vitamin B (such as vitamin B1 (thiamine), Vitamin B2 (riboflavin), vitamin B3 (niacin), vitamin B5 (pantothenic acid), vitamin B6 (pyridoxine), vitamin B7 (biotin), vitamin B9 (folic acid), and vitamin B12 (cobalamin), vitamin C (ascorbic acid), vitamin D (such as vitamin D2 (ergocalciferol) and vitamin D3 (cholecalciferol), vitamin E (such as α-, β-, γ-, and δ-tocopheryl, and α-, β-, γ-, and δ-tocotrienol)

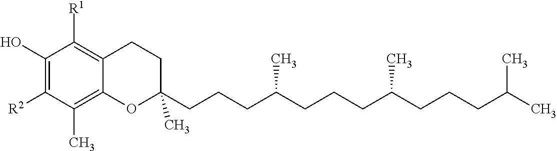

| | R¹ | R² |
|---|---|---|
| α-Tocopheryl | CH₃ | CH₃ |
| β-Tocopheryl | CH₃ | H |
| γ-Tocopheryl | H | CH₃ |
| δ-Tocopheryl | H | H |

or an acetate, succinate or nicotinate ester of any of the four foregoing,

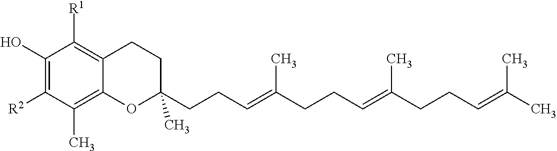

| | R¹ | R² |
|---|---|---|
| α-Tocopheryl | CH₃ | CH₃ |
| β-Tocopheryl | CH₃ | H |
| γ-Tocopheryl | H | CH₃ |
| δ-Tocopheryl | H | H |

or an acetate, succinate or nicotinate ester of any of the four foregoing, vitamin K, and an acetate, succinate or nicotinate ester of any of the foregoing, e.g., tocopheryl acetate (vitamin e acetate), preferably, wherein the amount of the sum of the vitamin(s) is between 0.0001% and 5% (w/w); An oral care composition according to the invention preferably comprises one or more vitamins. Vitamins can also include other vitamin-like compounds such as cholines, carnitines, or combinations thereof.

An oral care composition according to the invention preferably comprises between 0.0001% (w/w) to 5% (w/w) vitamins, such as from 0.01% (w/w) to 5% (w/w) or from 0.01% (w/w) to 2% (w/w) vitamins.

In one preferred embodiment, a composition comprise one or more (e9) retinoid compounds. As used herein, a “retinoid compound” includes all natural and/or synthetic analogs of vitamin A or retinol-like compounds that possess the biological activity of vitamin A in the skin, the geometric isomers and stereoisomers of these compounds as well as all natural and/or synthetic analogs of vitamin E. In one embodiment, (e9) comprises, preferably consists of, one or more retinoid compound(s) selected from the group consisting of tretinoin; isotretinoin; alitretinoin; acitretin; etretinate; motretinide; adapalene (6-3-(1-adamantyl)-4-methoxyphenyl-2-naphthoic acid); arotinoide; acetylenretinoide; tazarotene (ethyl 6-2-(4,4-dimethylthiochroman-6-yl)-ethynylnicotinate), retinyl esters such as retinyl acetate, retinyl palmitate, retinyl propionate, and retinyl linoleate; retinal; retinoic acid; tocopheryl acetate; and tocopheryl retinoate. A retinoid compound may be included as a substantially pure material, or in one an extract obtained by appropriate physical and/or chemical isolation from natural (e.g., plant) sources. The composition of this invention can contain a safe and effective amount of the retinoid compound so that the oral care composition is safe and effective to regulate or improve the condition of keratinous tissues and accidental insults as it is applied to the oral cavity. Preferably, the amount of the sum of the retinoid compound(s) is between 0.0001% (w/w) and 2% (w/w).

In one preferred embodiment, a composition comprise one or more (e10) Plant hormone preferably selected from jasmonic acid (JA) including jasmonates and conjugates thereof, gibberellins GA1 to GA136 including gibberellic acid and (GA1), ent-gibberellane, ent-kaurene (5,5,9-trimethyl-14-methylidenetetracyclo-[11.2.1.0^{1.10}.0^{4.9}]-hexadecane), and zeatin (2-methyl-4-(7H-purin-6-ylamino)but-2-en-1-ol) preferably, wherein the amount of the sum if these the amount of the sum of the compound is between 0.0001% and 1% (w/w). In one preferred embodiment, a composition comprises one or more JA(s) selected from the group consisting of methyljasmonate (MeJA—jasmonic acid methylester), JA conjugated with an amino acid selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, ptonith, phenylalanine, creatine, diaminobutonic acid, diaminopropionic acid, such as JA conjugated with isoleucine (Ile) (JA-Ile ((+)-7-iso-jasmonoyl-L-isoleucine)), and alkali and alkaline earth salts of JA, preferably, the amount of the sum of the one or more JA(s) is between 0.0001% (w/w) and 2% (w/w);

In one preferred embodiment, a composition comprise one or more (e11) amino acids including their salts and derivatives. In one preferred embodiment, a composition comprises one or more amino acid(s) and derivative thereof selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosin, valine, diaminobutyric acid, and diaminopropionic acid; and creatine, including Na, K, and Li salts of any of the foregoing and the acetate form of any of the foregoing, preferably, the amount of the sum of the one or more amino acid(s) is between 0.0001% (w/w) and 2% (w/w).

In one preferred embodiment, a composition comprise one or more (e12) oral cavity fortifying polyols (other than those in group (d)). Such polyols can be sugar alcohols, disaccharides, polysaccharides, and preferably non-reducing sugars. In one preferred embodiment, a composition comprises one or more polyol(s) selected from the group consisting of dexpanthenol (provitamin B5), cyclodextrins (e.g., (2-hydroxypropyl-β-cyclodextrin), mannitol, maltitol, lactitol, maltotritol, maltotetratol, polyglycitol, 1-O-α-D-Glucopyranosyl-D-mannitol, 6-O-α-D-Glucopyranosyl-D-glucitol, and isomalt, more preferably dexpanthenol, preferably, wherein the amount of the sum of the one or more polyol(s) is between 0.1% and 10% (w/w).

In one preferred embodiment, a composition comprise one or more (e13) Hydroxy acids including their salts and derivatives, preferably their Na, K and Li salts. In one preferred embodiment, a composition comprises one or more hydroxy acid(s) selected from the group consisting of acetic acid, propionic acid, salicylic acid, lactic acid, glycolic acid, acetylsalicylic acid and other salicylic acid derivatives, preferably salsalate (dimer of salicylic acid) and choline salicylate (salt of choline and salicylic acid), more preferably selected from the group consisting of salicylic acid, choline salicylate and salsalate, preferably, wherein the amount of the sum of the said preferred or more preferred hydroxy acid(s) is between 0.01% and 4%.

In one preferred embodiment, a composition comprise one or more (e14) Fluoride component. In one preferred embodiment, a composition comprises one or more fluoride component(s) selected from the group consisting of Na monofluorophosphate, amine fluoride und stannous fluoride, preferably, wherein the amount of the sum of the said fluoride ions of the fluoride components is between 0.0001% (w/w) and 0.5% (w/w), more preferably between 0.0001% (w/w) and 0.2% (w/w), even more preferably between 0.0001% and 0.15% (w/w) (i.e., only the amount of fluoride is taken into account when evaluating the amount of fluoride in a composition, the counter ion would be counted in group (e22). However, for evaluating the total amount of further components, preferably components (e), in a composition, the amount of the total fluoride component can be used (once);

In one preferred embodiment, a composition comprise one or more (e15) sweeteners other than the polyols (d). In one preferred embodiment, a composition comprises at least one sweetener selected from the group consisting of (Na) saccharin, dextrose, sucrose, lactose, levulose, aspartame, Na cyclamate, D-tryptophan, dihydrochalcone, acesulfame, sucralose, and neotame, preferably, wherein the amount of the sum of the sweeteners is between 0.1% and 10% (w/w);

In one preferred embodiment, a composition comprise one or more (e16) colorants. The skilled person will understand that colorants can already be part of plant oils or extracts, or propolis or can be added independently to a composition in accordance with the invention. The term colorant also encompasses oral cavity acceptable anti dye, e.g., Na hexametaphosphate. In one preferred embodiment, a colorant is selected from the group consisting of chlorophyll, indigo, beta-carotene, saffron, paprika, green S, patent blue V, quinoline yellow, carmoisine, ponceau 4R, and Na hexametaphosphate preferably, the amount of the sum of colorants is between 0.0001% and 1% (w/w).

In one preferred embodiment, a composition comprise one or more (e17) peptides. The three letter code for amino acids is known to the skilled person. The term peptides encompasses, fatty acid derivatives, peptide acetates, Na, Li and K salts of said peptides. In one preferred embodiment, a composition comprises one or more peptide(s) selected from the group consisting of carnosine (Beta-alanin and L-histidin; Ala-His), Gly-His-Lys, Arg-Lys-Arg, His-Gly-Gly; palmitoyl-Gly-His-Lys (commercially available e.g. from Sederma, France), Arg-Lys-Arg, Peptide E (Arg-Ser-Arg-Lys), Lys-Thr-Thr-Lys-Ser, palmitoyl-Lys-Thr-Thr-Lys-Ser (commercially available from Sederma, France), fatty acid derivatives, acetates, Na salts, Li salts and K salts of any of the foregoing, preferably, the amount of the sum of these peptides is between 0.0001% and 2% (w/w).

Sensations such as cooling, warming, and tingling are useful in providing signals to the user.

In one preferred embodiment, a composition comprise one or more (e18) cooling agents. The best-known coolant is menthol. It can, in particular 1-menthol, therefore, additionally be added to an oral care composition according to the invention. Among the synthetic coolants are many derivatives structurally related to menthol, i.e., comprising a cyclohexane moiety which is derivatized with functional groups such as carboxamide, ketal, ester, ether and alcohol. Examples include the p-menthanecarboxamide compounds such as N-ethyl-p-menthane-3-carboxamide (commercially known as “WS-3”). An example of a synthetic carboxamide coolant structurally unrelated to menthol is N,2,3-trimethyl-2-isopropylbutanamide. Other exemplary synthetic refrigerants are alcohol derivatives such as 3-1-menthoxy-propane-1,2-diol, isopulegol, p-menthane-3,8-diol, menthoneglycerol acetal (commercially known as “MGA”), menthyl esters (such as menthyl acetate, menthyl acetate, menthyl acetoacetate and monomenthyl lactate), alpha-ketoenamine derivatives including 3-methyl-2-(1-pyrrolidinyl)-2-cyclopenten-1-one (3-MPC),5-methyl-2-(1-pyrrolidinyl)-2-cyclopenten-1-one(5-MPC);2,5-dimethyl-4-(1-pyrrolidinyl)-3(2H)-furanone(DMPF); icilin (aka AG-3-5, chemical name142-hydroxyphenyl]-4-[2-nitrophenyl]-1,2,3,6-tetrahydropyrimidin-2-one), preferably, the amount of the sum of these cooling agents is between 0.0001% and 2% (w/w).

In one preferred embodiment, a composition comprise one or more (e19) Warming agents, e.g., nicotinic esters (such as benzyl nicotinate), polyhydric alcohols, nonanoyl vanillylamide, nonanoic acid vanillyl ether, vanillyl alcohol alkyl ether derivatives (such as vanillyl ethyl ether, vanillyl butyl ether, vanillyl pentyl ether, vanillyl hexyl ether), isovanillyl alcohol alkyl ethers, ethyl vanillyl alcohol alkyl ethers, veratryl alcohol derivatives, substituted benzyl alcohol derivatives, substituted benzyl alcohol alkyl ethers, vanillin propylene glycol—Acetal, ethyl vanillin propylene glycol acetal, ginger extract, ginger oil, gingerol, zingerone, or combinations thereof, preferably, the amount of the sum of these warming agents is between 0.0001% (w/w) and 2% (w/w). Although e.g., ethanol and other short-chained (poly)hydric alcohols are also heating agents, these are attributed to the protic solvents (a) or, in case of xylitol, erythritol and sorbitol, attributed to polyols (d), not to the further components (e). Moreover, the skilled person understands components with multiple effects (e.g., a polyhydric alcohol can be sweeteners and warming agents), are only counted once in group (e).

In one preferred embodiment, a composition comprise one or more (e20) tingling agents, e.g., capsaicin, homocapsaicin, jambuoleoresin, zanthoxylumpeperitum, saanshool-I, saanshoolll, sanshoamide, piperine, piperidine, spilanthol, 4-(1-methoxymethyl)-2-phenyl-1,3-dioxolane, or combinations thereof, preferably, the amount of the sum of these tingling agents is between 0.0001% (w/w) and 2% (w/w).

In one preferred embodiment, a composition comprise one or more (e21) emulsifier (compounds which help to combine liquids of different thicknesses). Emulsifiers usually have a hydrophilic and a hydrophobic residue. When they are added to an unmixable liquid, the emulsifier molecules position themselves along the so-called interfacial layer where, e.g., oil separates from water. Preferred but not limiting examples are sodium diphosphate (Na₄P₂O₇), PEG300, PVPK90, lecithin, carrageenen, guar gum, xanthan gum, polysorbate (e.g., polyoxyethylen(20)-sorbitan-monolaurat (polysorbat 20), polyoxyethene (20) sorbitan-monooleate (polysorbat 80), polyoxyethene (20) sorbitan-monopalmitate (polysorbat 40), polyoxyethene (20) sorbitan-monostearate (polysorbat 60), polyoxyethene (20) sorbitan-monotristearate (polysorbat 65)), celluloses (e.g. cellulose, methyl cellulose, ethyl cellulose, hydroxypropyl cellulose, hydroxypropylmethylcellulose, ethyl methyl cellulose, cellulose, (sodium) carboxymethylcellulose, ethyl hydroxyethyl cellulose, Na laurylsulfate (SDS), crosslinked Na carboxymethyl cellulose (Croscarmellose), enzymatically hydrolysed carboxymethylcellulose (E469), mono- and diglycerides of fatty acids (e.g. glyceryl monostearate, glyceryl distearate) sucrose esters and sucroglycerides (E473 and E474), polyglycerolesters of fatty acids, polyglycerol polyricinoleate, stearoyl lacylates (e.g. sodium or calcium stearoyl-2-lactylate), sorbitan esters (e.g., sorbitan monostearate, sorbitan tristearate, sorbitan monolaurate, sorbitan monooleate, sorbitan monopalmitate). Preferably, the amount of the sum of these emulsifiers is between 0.0001% (w/w) and 5% (w/w).

A composition may comprise further additives (e22) either as additives to commercial products which are used to provide a component (a) to (e) or are added due to a beneficial effect, such as metal ion providing sources, preferably NaCl, or Ca²⁺, Mg²⁺, Zn²⁺, Fe^{2+/3+} providing sources. Metal ion sources can be, e.g., tea, cocoa and lotus extracts or specific addition of metal salts. The skilled person is aware which salts are sufficient water-soluble to provide the metal ions to the gum/teeth surface, such salts are, e.g., calcium acetate, calcium citrate, calcium lactate, CaCl₂, CaNO₃, FeCl₂, FeCl₃, Fe₂(SO₄)₃, MgCl₂, MgSO₄, magnesium acetate, zinc acetate, zinc nitrate, ZnSO₄, ZnCl, zinc chlorate (Zn(ClO₃)₂ and zinc perchlorate (CL₂O₂Zn), zinc gluconate, and zinc acetate. If a metal ionproviding source is specifically added in form of a salt, the amount of such salts are between 0.001% (w/w) and 2.5% (w/w), more preferably between 0.01% (w/w) and 1% (w/w). Notably, the anion of such a salt would be counted into group (e22), as well, to evaluate the total amount of (e). The term “further additives” encompasses all substances which are not mentioned, in any of the prominent classes (a) to (d) and (e1) to (e22) but which may be present in a composition according to the invention in amounts of 1% (w/w) or less, preferably (except for metal ion providing sources) 0.05% (w/w) or less.

The skilled person understands that further components in addition to components (a) to (d) may have further beneficial effects to a composition but the good results of the compositions according to the invention are based on the presence of components (a) to (d) in their specific amounts, in a preferred embodiment, (a) to (d) and (e4) with their specific amounts described herein.

### Oral Care Composition

In a preferred embodiment, the “contact time” (the time in which at least 10% of a composition according to the invention is still on the surface of a mucous membrane, gum or tooth or the inside of a worn aligner splint) after it has been applied is more than 2 minutes, preferably more than 10 minutes, preferably more than 30 minutes, more preferably 60 minutes or longer, such as up to 3 hours, up to 6 hours, up to 8 hours or longer than 8 hours.

If an (aligner) splint is used, a composition according to the invention can be applied to the inside of the (aligner) splint (e.g., three doses, one in the front area and one on each side of the splint) before the (aligner) splint is inserted, so that when the (aligner) splint is inserted a composition according to the invention is distributed and fixed between the (aligner) splint and gum or tooth via volume displacement. Alternatively, a composition according to the invention can be applied to the teeth and gum in question, e.g., shortly before insertion of the aligner splint or to the tissue and teeth around a dental implant The use of a splint such as an aligner splint or a splint designed to retain a formulation for a longer period of time in the oral cavity or a mouth guard increases the residence time of the composition and thus the exposure time by reducing salivation of the composition. In such a use of a composition according to the invention, the exposure time is usually 60 minutes or longer, preferably up to 3 hours, more preferably up to 6 hours, even more preferably up to 8 hours or even longer than 8 hours, depending on the wearing time of the (aligner) splint. It is desirable to use a gel for the present invention that allows for easy application, thin layering, and even distribution in the gingival sulcus/pockets and long gingival gum line.

The oral care composition according to the invention can be administered as a(n) (oral) spray, gel (e.g., hydrogel), emulsion, dispersion, as part of a dental strip, or chewing gum, preferably, the oral care composition of the invention is a hydrogel. Examples of oral care compositions include tooth/gingival gel, subgingival gel, dispersion or emulsion for application to teeth, gums and/or aligner splints. Compositions of the inventions can also be used to prepare mouthwash, oral spray, chewable tablets, chewing gum, dental strips, dental floss and dental floss coatings, breath freshening dissolvable strips, prosthesis care products, dental care products, in particular aligner splint care products. Also described herein is a method of using the disclosed compositions to improve gum health in the oral cavity, preferably when wearing splints such as an aligner splint or a mouth guard, or using a composition of the invention to care for the gum when wearing a splint, preferably an aligner splint or a mouth guard. The user can apply the composition to the teeth and/or gums and/or an aligner themselves, or the composition can be applied by a third party, e.g., a dentist, an orthodontist or other medical or dental professionals.

### Preparation Methods

The compositions of the present invention can be prepared by simply mixing most of the components. Preferably, HA is first dispersed in ethanol (e.g., 96%) or in an ethylene glycol comprising medium and then the aqueous phase is added under stirring. Alternatively, HA can be added to an ethanol or ethylene glycol comprising product such as a carbomer before adding the aqueous phase.

Further components can be added either to the aqueous phase prior to mixing it with a HA dispersion or can be added after the (suspended) HA and the aqueous phase were combined, or some components are added to the (ethanolic/ethylene glycol) HA dispersion and others to the aqueous phase prior to mixing it with HA or can be added after the (suspended) HA and the aqueous phase were combined. Antioxidants such as catechins are preferably added to a composition according to the invention, preferably in the form of plant extract, e.g., a tea extract. Further beneficial components such as vitamins, minerals and other secondary plant substances can also be added directly as further components (e) or in form of components of an extract, e.g., a lotus extract. Lotus extract is also preferably added to a composition according to the invention. Rich in armepavin, fatty acid, protein, phosphorus acid and linoleic acid, it has a beneficial effect on the surface of the gums. In addition to fiber and vitamins, lotus extract also comprises important minerals such as compounds with iron, copper, manganese, zinc and K ions.

Furthermore, the pH of a composition is preferably between 6 and 8. This can be done with any pharmaceutically acceptable buffer compound or the sum of all components of a composition already provide an ambient pH value. In a preferred embodiment, the pH is adjusted with a buffer(system) selected from the group consisting of sodium bicarbonate, TRIS and PBS. A pH below pH 6 accelerates the demineralization of the teeth, a pH above 8 is too far from the pH of the human oral cavity, which should be between 6 and 7.5. If the pH value becomes too high, it can lead to chemical burns in the oral cavity. The dispersion as well as the mixing procedure can be performed preferably around 298 K, e.g., at 298.15±25 K but also higher temperature such as up to 80° C. are possible (unless components are heat sensitive). Preferably, the pressure is around 1.013 bar, e.g., 1.013±200 bar.

A method for preparing a composition according to the invention comprises the steps:
optionally calculating the amounts of water-insoluble polymers to obtain a suitable viscosity as disclosed herein;
disperse HA in ethanol or ethylene glycol or ethanol- or ethylene glycol-comprising solvents;
adding the aqueous phase wherein part or all other components are already dissolved in the aqueous phase, optionally, adding components after the aqueous phase (with or without parts of the components) is already combined with the dispersed HA and stir until the hydrogel is formed, preferably at least one hour;
optionally measuring the pH of the composition;
optionally adjusting the pH to a range between pH 6 and pH 7.5, preferably in the range of pH 7 (e.g., pH 7±0.3);
optional measuring the viscosity (e.g., for quality management).

Those skilled in the art will appreciate that once a composition has been formulated correctly, calculating the amounts of water-insoluble polymers and protic solvents to obtain a suitable viscosity and/or viscosity measurement are no longer required in an existing recipe; also pH measurement and pH adjustment may no longer be required in an existing recipe. For quality management reasons, however, at least the steps of measuring the pH value and, if necessary, adjusting the pH value and/or measuring the viscosity should continue to take place.

### Uses

The present invention also relates to methods of caring for the oral cavity, especially the gum and/or teeth, comprising applying a composition according to the invention to the intraoral tissue (e.g. oral mucosa, gingiva) of the oral cavity or to the teeth of a user, preferably to the gum (or part of the gum) of a user, with an exposure time of at least 30 seconds, preferably more than 2 minutes, more preferably more than 10 minutes, even more preferably more than 30 minutes or longer. If a splint such as an aligner splint, a mouth guard or a splint designed to encompass (part of) the gum of a user (i.e. individually adjusted to the denture of a user) is worn, the exposure time is usually 60 minutes or longer, preferably up to 3 hours, more preferably up to 6 hours, even more preferably up to 8 hours or even longer than 8 hours, depending on the wearing time of the aligner splint. By pressing the composition according to the invention between the gingiva, teeth and (aligner) splint, the diffusion of the composition and the dilution of the composition by saliva is notably delayed/prevented. The method of application described herein comprises contacting a person's oral mucosa (e.g., gum line or periodontal pockets) with the oral care composition of the invention. The regions of the gum and the teeth that are in the inner area of a splint (such as an aligner splint) when a splint is worn, should preferably be brought into contact with a composition according to the invention. If an aligner splint is worn, a composition according to the invention can be applied to the inside of the aligner splint before the aligner splint is inserted, so that when the aligner splint is inserted it is fixed between splint and gum or teeth via volume displacement. Alternatively, a composition according to the invention can be applied to the teeth and gum in question shortly before insertion of the aligner splint. This also increases the residence time of the composition and thus the exposure time. In such a use of a composition according to the invention, the exposure time is usually 60 minutes or longer, preferably up to 3 hours, more preferably up to 6 hours, even more preferably up to 8 hours or even longer than 8 hours, depending on the wearing time of the aligner splint. “Shortly” before inserting a(n) (aligner) splint preferably means at most three minutes before inserting a(n) (aligner) splint, more preferably at most one minute, even more preferably at most 30 seconds before inserting a(n) (aligner) splint. A composition according to the invention is preferably applied to the inside of a splint such as a mouthguard, individually adapted splint or aligner splint or the corresponding regions of the oral cavity each time before a splint such as a mouthguard, individually adapted splint (i.e. a splint to enhance retention time of a composition but which is not designed to change the position of teeth) or aligner splint is inserted. Of course, other regions of the oral cavity can also be brought into contact with the composition according to the invention.

The present invention further relates to a method of improving a user's gum health, comprising the step of applying the oral care composition to a user's intraoral tissue, preferably along the gumline or sulcus.

One aspect of the invention refers to the use of a composition according to aspect one or any embodiment of aspect one for the treatment of the gum and/or teeth covered by an aligner splint when the latter is worn. Another aspect refers to a method for the treatment of gum and teeth comprising the steps: a) applying a composition according to aspect one or any embodiment thereof to the gum and teeth covered by an aligner splint when the latter is worn; or applying a composition according to aspect one or any embodiment thereof to the inside of an aligner splint which, when worn, encompasses a portion of a wearer's gum and teeth; b) placing the aligner splint in the oral cavity and wear the aligner splint for at least 60 (sixty) minutes. A composition according to the invention can also be applied to the skin (cutis) outside the mouth area.

Another aspect relates to a composition according to aspect one or any embodiment thereof as a medicament.

A further aspect relates to a composition according to aspect one or any embodiment thereof for use in the treatment of inflammation, preferably of the cutis, more preferably the oral mucosa, even more preferably the gingiva; preferably the inflammatory is periodontitis. This also refers to the treatment of inflammation of tissue close to an implant.

Another aspect relates to a composition according to aspect one or any embodiment thereof for use in the treatment of canker sores.

Another aspect relates to a composition according to aspect one or any embodiment thereof for use in the treatment of gum recession.

A further aspect relates to the use of a composition according to aspect one or any embodiment thereof for preventing inflammatory of the gum.

A further aspect refers to the use of a composition according to the invention for the treatment of hypersensitivity of teeth or tooth necks.

A further aspect refers to the use of a composition according to the invention for the treatment of injuries, preferably small injuries of the cutis, preferably the oral mucosa, in particular the gingiva (preferably cuts or abrasions in the oral mucosa that are no longer than 0.5 cm or whose longest extent is no longer than 0.5 cm) can be used.

The invention therefore also relates to a method for treating inflammatory; or canker sores; or gum recession; or hypersensitivity of teeth or tooth necks; or injuries of the cutis, preferably the oral mucosa, which comprises the step of applying a composition according to aspect one or any embodiment thereof to the respective surface at least one time within a period of 12 hours, more preferably two, three, four or five times or even more within a time period of 12 hours.

In one preferred embodiment, a composition according to the invention is used in combination with a delivery carrier such as a strip, a dental splint (including an aligner splint) or sponge material for any of the uses or methods described above.

This application of a composition according to the invention preferably takes place at least once a day, preferably at least twice a day, even more preferably at least three times a day, for all the uses and methods indicated herein. In general, however, a composition according to the invention should not be used more than 15 times, preferably no more than 10 times, even more preferably no more than 6 times per day (i.e., within 24 h).

The composition of the present invention can be applied with a syringe or one-time syringe, a squeeze tube, a brush, a penor or brush tip applicator, a swab, a lip gloss applicator, a toothbrush, a strip that is removed after use, or the like, or even with the fingers.

After the desired time has elapsed, any residue can be easily removed by wiping, brushing or rinsing the (oral) surface. Alternatively, the remaining composition can be left in contact with the oral surface. The compositions according to the invention will disappear over the time from the surface of the teeth/oral mucosa, in particular the gingiva, due to diffusion, movements in the mouth and/or salivation.

A further aspect of the present invention relates to a kit comprising a container with a composition according to the invention, which is suitable for applying the composition to the appropriate regions of the oral cavity or a splint and instructions on how to use a composition according to the invention.

### EXAMPLES

Commercially available components were used to prepare the compositions. The following examples demonstrate the compositions and uses of the composition of the invention but do not limit the concept of this invention to these examples,

a)
water (CG, PRO, CE(I)), tap water
Glycerol (99.7%) (Labordiscounter, the Netherlands)
ethanol (EtOH) (source from CG, PRO, CE1 see below, and Roth, Karlsruhe, Germany)
Propylene glycol (propane-1,2-diol) (CG, see below)
Pentylene glycol (Merck, Karlsruhe, Germany)
   b)
Carbomer gel pH 6.5 NRF S43 (Carbomer 35.000 1 g, trometamol (Tris(hydroxymethyl)aminomethane (TRIS)) 1 g, sodium edetate (sodium ethylenediaminetetraacetate (EDTA)) 0.1 g, propylene glycol 10 g, water 100 g) from Caesar & Lorent GmbH, Hilden, Germany (CG)
D-Galacto-D-mannan from Ceratonia siliqua (CAS 11078-30-1) (mannose/galactose ratio 3.4:1), low substituted hydroxypropyl cellulose (CAS 9004-64-2), carboxymethylcellulose Na salt (CAS 9004-32-4) from Merck Sigma Aldrich, Darmstadt, Germany
D-Galacto-D-mannan from Caesalipina spinosa (CAS 39300-88-4) (mannose/galactose ratio 3:1) from Dragonspice Naturwaren, Reutlingen, Germany
Na hyaluronate (HA I) 8000-12000 Da (HA I) from Laboratories Hyamed SA, Geneva, Switzerland
Na hyaluronate 10000 to 50000 Da (HA II) from Alexmo cosmetics GmbH, Stuhr, Germany
Na hyaluronate 1200 to 1800 kDa (HA III) from ExperChem Limited Weinheim, Germany
   c)
Chamomile extract (1:4, 40% ethanol-alpha bisabolol content 1.44%, chamazulene content 0.9% in extract) from Viatris, Steinhausen, Switzerland (CE I)
Chamomile extract (CO₂ extraction-alpha bisabolol content 1.4%, matricin content 0.51%) from Flavex Naturextracts GmbH, Rehlingen-Siersburg, Germany (CE II)
Alpha-bisabolol, eugenol, curcumin Beta-caryophyllene ((−)-trans-Caryophyllene, trans-(1R,9S)-8-Methylene-4,11,11-trimethylbicyclo[7.2.0]undec-4-ene Empirical Formula (Hill Notation)) (CAS 87-44-5), chlorhexidine (CAS 66-56-1), hexadecylpyridinium chloride monohydrate (CAS 6004-24-6) from Merck Sigma Aldrich, Darmstadt, Germany
Matricin from BioCrick, Chengdu, China
Eugenol (Speiko, Munster, Germany)
   d)
   Xylitol, Erythritol, Sorbitol from Merck Sigma Aldrich, Darmstadt, Germany
   e)
Propolis 1.2% Propolis extract in 40% ethanol from Hoyer GmbH, Polling, Germany (PRO)
Catechin 90% from lyophilized green tea extract with a distribution of EC:ECG:EGC:EGCG of 5:9:14:31) from NatuGena GmbH, Ingolstadt, Germany (Catechin)
EGCG Hydrate (CAS 989-51-5) purity>98.0% HPLC and EC (CAS 490-46-0) purity>97.0% HPLC from TCI Zwijndrecht, Belgium
Mentha arvensis leaf oil 63% menthol content (Essence-pur GmbH Lohmar, Germany)
allantoin, 2-phospho-L-ascorbic acid trisodium salt (sodium ascorbyl phosphate), beta-carotene, stanous fluoride (SnF₂), retinol, menthol, methylparabene, sucrose-monolaurate, sodium laurylsulfate (SDS) (Merck Reutlingen, Deutschland)
dexpanthenol provitamin B5, (Labordiscounter, the Netherlands)
NaOH (Labordiscounter, the Netherlands) was used to prepare a 50% NaOH solution
PBS Buffer pH 7.4-10× concentrate (Morphisto GmbH Offenbach, Germany) diluted 1:9 (1×PBS with 8,184 g NaCl, 1.8 g Na₂HPO₄ and 0.3 g KH₂PO₄)
sodium saccharin (Freyalab Hadzhidimovo, Bulgaria)
Polyethylenglycol PEG300, Polyvinalpyrrolidon PVP K90, sodium diphosphate (Na₄P₂O₇) and sodium bicarbonate (NaHCO₃), tocopheryl acetate (vitamine e acetate) (Roth, Karlsruhe, Germany)
N-Ethyl-p-menthane-3-carboxamide (TCI Chemical, Eschborn, Deutschland)
Chlorhexamed 1% Gel (GlaxoSmithKline GmbH, Munchen, Germany) (1.13 mg chlorhexidine/0.2 g gel=0,565% (w/w); further ingredients: 2-propanol, hyprolose, sodium acetate, macrogol glycerolhydroxystearate, water).

Compositions with CG were prepared in that CG and further EtOH containing components (e.g., PRO and CE I) were combined and HA was added in small portions under stirring for 2 h at room temperature until a homogeneous dispersion was formed. Water was added in small portions (dropwise) under stirring and then further components were added under stirring.

For compositions with D-Galacto-D-mannan, hydroxypropyl cellulose or sodium carboxymethylcellulose, HA was first dispersed in EtOH 96 Vol.-% under stirring. Then further components comprising EtOH were added dropwise, then D-galacto-D-mannan hydroxypropyl cellulose or sodium carboxymethylcellulose was added in small portions. After 2 h stirring at room temperature, water and further components were added under stirring dropwise/as solids. Solid (water soluble) solids were either first dissolved in water and then the respective amount of a solution was added to the composition (for calculation's sake, the amount of water was added to the amount of water under item (a) in the tables below) or the solids were added to the combined HA and aqueous phase and the resulting compositions were stirred until all solids were dissolved.

### Example 1: CE I

| Ingredient | Amount in g | Amount in % |
|---|---|---|
| (a) water (CG) | 2.68 | |
| (a) ethanol (PRO) | 0.08 | |
| (a) ethanol (CE I) | 0.05 | |
| (a) water (CE I) and (PRO) | 0.19 | |
| (a) propane-1,2-diol (CG) | 0.27 | |
| Total (a): | 3.27 | 66.21 |
| (b) hyaluronic acid (HA I) | 0.07 | 1.41 |
| (b) carbomer 35000 (CG) | 0.03 | |
| Total (b): | 0.1 | 2.02 |
| (c) xylitol | 1.5 | 30.37 |
| (d) chamazulene (CE I) | 0.0015 | |
| (d) alpha-bisabolol (CE I) | 0.0024 | 0.05 |
| Total (d): | 0.0039 | 0.08 |
| (e2) TRIS (CG) | 0.03 | |
| (e3) EDTA (CG) | 0.003 | |
| (e1) further ingredients (CE I) | 0.02943 | |
| (e1) PRO | 0.0024 | |
| Total (e): | 0.06483 | 1.31 |
| Total composition: | 4.93873 | |

pH 6.2

### Example 1a: CE I+Catechin

| Ingredient | Amount in g | Amount in % |
|---|---|---|
| (a) water (CG) | 2.58 | |
| (a) ethanol (PRO) | 0.08 | |
| (a) ethanol (CE I) | 0.05 | |
| (a) water (CE I) and (PRO) | 0.19 | |
| (a) 1,2-propanediol (CG) | 0.27 | |
| Total (a): | 3.17 | 64.19 |
| (b) hyaluronic acid (HA I) | 0.07 | 1.34 |
| (b) carbomer 35000 (CG) | 0.03 | |
| Total (b): | 0.1 | 2.02 |
| (c) xylitol | 1.5 | 30.37 |
| (d) chamazulene (CE I) | 0.0015 | |
| (d) alpha-bisabolol (CE I) | 0.0024 | |
| Total (d): | 0.0039 | 0.08 |
| (e2) TRIS (CG) | 0.03 | |
| (e3) EDTA (CG) | 0.003 | |
| (e1) further ingredients (CE I) | 0.02943 | |
| (e1) PRO | 0.0024 | |
| (e4) catechin | 0.09 | |
| (e1) further ingredients (catechin) | 0.01 | |
| Total (e): | 0.16483 | 3.34 |
| Total composition: | 5.03873 | |

pH 6.4

### Example 2: CE II+Catechin+Lotus

| Ingredient | Amount in g | Amount in % |
|---|---|---|
| (a) water | 3.00 | |
| (a) water (PRO) | 0.19 | |
| (a) ethanol (PRO) | 0.08 | |
| Total (a): | 3.27 | 64.5 |
| (b) hyaluronic acid (HA I) | 0.07 | 1.38 |
| (b) D-Galacto-D-Mannan | 0.05 | 0.99 |
| Total (b): | 0.12 | 2.37 |
| (c) Xylitol | 1.5 | 29.57 |
| (d) matricin (CE II) | 0.0001 | 0.002 |
| (d) alpha-bisabolol (CE II) | 0.00028 | 0.006 |
| Total (d): | 0.00038 | 0.008 |
| (e1) lotus extract (lyophilisate) | 0.05 | |
| (e4) catechin | 0.09 | |
| (e1) further ingredients (catechin) | 0.01 | |
| (e1) further ingredients (CE II) | 0.01962 | |
| (e2) sodium bicarbonate | 0.01 | |
| (e1) PRO | 0.0024 | |
| Total (e): | 0.18202 | 3.59 |
| Total composition: | 5.0724 | |

pH 7.0

### Example 3: (not According to the Invention)

| Ingredient | Amount in g | Amount in % |
|---|---|---|
| (a) water | 3.00 | |
| (a) water (PRO) | 0.19 | |
| (a) ethanol (PRO) | 0.08 | |
| Total (a): | 3.27 | 85.55 |
| (b) hyaluronic acid (HA I) | 0.07 | 1.83 |
| (b) D-Galacto-D-Mannan | 0.05 | |
| Total (b): | 0.12 | 3.14 |
| (c) xylitol | 0.3 | 7.85 |
| (d) matricin (CE II) | 0.0001 | |
| (d) alpha-bisabolol (CE II) | 0.00028 | |
| Total (d): | 0.00038 | 0.01 |
| (e4) catechin | 0.09 | |
| (e1) further ingredients (catechin) | 0.01 | |
| (e2) sodium bicarbonate | 0.01 | |
| (e1) PRO | 0.0024 | |
| (e1) further ingredients (CE II) | 0.01962 | |
| Total (e): | 0.13202 | 3.45 |
| Total composition: | 3.8224 | |

pH 7.1

### Example 4: CE II+Catechin+Lotus

| Ingredient | Amount in g | Amount in % |
|---|---|---|
| (a) water | 3.00 | |
| (a) water (PRO) | 0.19 | |
| (a) ethanol (PRO) | 0.08 | |
| Total (a): | 3.27 | 65.17 |
| (b) hyaluronic acid (A I) | 0.07 | 1.40 |
| (c) xylitol | 1.5 | 29.90 |
| (d) matricin (CE II) | 0.0001 | |
| (d) alpha-bisabolol (CE II) | 0.00028 | |
| Total (d): | 0.00038 | 0.008 |
| (e1) lotus extract (lyophilisat) | 0.05 | |
| (e4) catechin | 0.09 | |
| (e1) further ingredients (catechin) | 0.01 | |
| (e2) sodium bicarbonate | 0.005 | |
| (e1) PRO | 0.0024 | |
| (e1) further ingredients (CE II) | 0.01962 | |
| Total (e): | 0.17702 | 3.53 |
| Total composition: | 5.0174 | |

pH 6.9

### Example 5: CE II+Catechin

| Ingredient | Amount in g | Amount in % |
|---|---|---|
| (a) water | 3.00 | |
| (a) water (PRO) | 0.19 | |
| (a) ethanol (PRO) | 0.08 | |
| Total (a): | 3.27 | 65.76 |
| (b) hyaluronic acid (HA I) | 0.07 | 1.41 |
| (c) xylitol | 1.5 | 30.17 |
| (d) matricin (CE II)) | 0.0001 | |
| (d) alpha-bisabolol (CE II) | 0.00028 | |
| Total (d): | 0.00038 | 0.008 |
| (e4) catechin | 0.09 | |
| (e1) further ingredients from Catechin | 0.01 | |
| (e2) sodium bicarbonate | 0.01 | |
| (e1) PRO | 0.0024 | |
| (e1) further ingredients (CE II) | 0.01962 | |
| Total (e): | 0.13202 | 2.66 |
| Total composition: | 4.9724 | |

pH 7.0

### Example 6: (not According to the Invention)

| Ingredient | Amount in g | Amount in % |
|---|---|---|
| (a) water | 5.00 | |
| (a) water (PRO) | 0.19 | |
| (a) ethanol (PRO) | 0.08 | |
| Total (a): | 5.27 | 75.65 |
| (b) hyaluronic acid (HA (I) | 0.02 | 0.29 |
| (c) xylitol | 1.5 | 21.53 |
| (d) matricin (CE II) | 0.0001 | |
| (d) alpha-bisabolol (CE II) | 0.00028 | |
| Total (d): | 0.00038 | 0.005 |
| (e1) lotus extract (lyophilisat) | 0.05 | |
| (e4) catechin | 0.09 | |
| (e1) further ingredients (catechin) | 0.01 | |
| (e2) sodium bicarbonate | 0.004 | 0.06 |
| (e1) PRO | 0.0024 | |
| (e1) further ingredients (CE II) | 0.01962 | |
| Total (e): | 0.17602 | 2.53 |
| Total composition: | 6.9664 g | |

pH 7.0

### Example 7: Chem (d)

| Ingredient | Amount in g | Amount in % |
|---|---|---|
| (a) water | 3.02 | |
| (a) water (PRO) | 0.19 | |
| (a) ethanol (PRO) | 0.08 | |
| Total (a): | 3.29 | 66.83 |
| (b) hyaluronic acid (HA I) | 0.07 | 1.42 |
| (b) D-Galacto-D-Mannan | 0.05 | 1.02 |
| Total (b): | 0.12 | 2.44 |
| (c) xylitol | 1.5 | 30.47 |
| (d) alpha-Bisabolol (SigmaAldrich) | 0.0003 | |
| (d) matricin (BioCrick) | 0.0001 | |
| Total (d): | 0.0004 | 0.01 |
| (e2) sodium bicarbonate | 0.01 | 0.2 |
| (e1) PRO | 0.0024 | |
| Total (e): | 0.0124 | 0.25 |
| Total composition: | 4.9228 | |

pH 7.0

### Example 7a: Chem d+Catechin+Xylitol

| Ingredient | Amount in g | Amount in % |
|---|---|---|
| (a) water | 3.02 | |
| (a) water (PRO) | 0.19 | |
| (a) ethanol (PRO) | 0.08 | |
| Total (a): | 3.29 | 65.45 |
| (b) hyaluronic acid (HA I) | 0.07 | 1.39 |
| (b) D-Galacto-D-Mannan | 0.05 | |
| Total (b): | 0.12 | 2.39 |
| (c) xylitol | 1.5 | 29.84 |
| (d) alpha-Bisabolol (SigmaAldrich) | 0.0003 | |
| (d) matricin (BioCrick) | 0.0001 | |
| Total (d): | 0.0004 | 0.01 |
| (e2) sodium bicarbonate | 0.014 | 0.28 |
| (e1) PRO | 0.0024 | |
| (e4) catechin | 0.09 | |
| (e1) further ingredients (catechin) | 0.01 | |
| Total (e): | 0.1164 | 2.32 |
| Total composition: | 5.0268 | |

pH 7.1

### Example 7b: Chem d+Catechin+Sorbitol

| Ingredient | Amount in g | Amount in % |
|---|---|---|
| (a) water | 3.02 | |
| (a) water (PRO) | 0.19 | |
| (a) ethanol (PRO) | 0.08 | |
| Total (a): | 3.29 | 65.45 |
| (b) hyaluronic acid (HA I) | 0.07 | 1.39 |
| (b) D-Galacto-D-Mannan | 0.05 | |
| Total (b): | 0.12 | 2.39 |
| (c) sorbitol | 1.5 | 29.84 |
| (d) alpha-Bisabolol (SigmaAldrich) | 0.0003 | |
| (d) matricin (BioCrick) | 0.0001 | |
| Total (d): | 0.0004 | 0.01 |
| (e2) sodium bicarbonate | 0.014 | 0.28 |
| (e1) PRO | 0.0024 | |
| (e4) catechin | 0.09 | |
| (e1) further ingredients (catechin) | 0.01 | |
| Total (e): | 0.1164 | 2.32 |
| Total composition: | 5.0268 | |

pH 7.0

### Example 7c: Chem d+Catechin+Erythritol

| Ingredient | Amount in g | Amount in % |
|---|---|---|
| (a) water | 3.02 | |
| (a) water (PRO) | 0.19 | |
| (a) ethanol (PRO) | 0.08 | |
| Total (a): | 3.29 | 65.45 |
| (b) hyaluronic acid (HA I) | 0.07 | 1.39 |
| (b) D-Galacto-D-Mannan | 0.05 | |
| Total (b): | 0.12 | 2.39 |
| (c) erythritol | 1.5 | 29.84 |
| (d) alpha-Bisabolol (SigmaAldrich) | 0.0003 | |
| (d) matricin (BioCrick) | 0.0001 | |
| Total (d): | 0.0004 | 0.01 |
| (e2) sodium bicarbonate | 0.014 | 0.28 |
| (e1) PRO | 0.0024 | |
| (e4) catechin | 0.09 | |
| (e1) further ingredients (catechin) | 0.01 | |
| Total (e): | 0.1164 | 2.32 |
| Total composition: | 5.0268 | |

pH 7.0

### Example 8: Bisabolol 0.01

| Ingredient | Amount in g | Amount in % |
|---|---|---|
| (a) water (3.26 g) + EtOH (0.01 g) | 3.27 | 67.14 |
| (b) hyaluronic acid (HA II and HA III 1:1) | 0.07 | 1.44 |
| (c) xylitol | 1.5 | 30.8 |
| (d) alpha-bisabolol | 0.0003 | 0.01 |
| (e) sodium bicarbonate | 0.03 | 0.62 |
| Total composition: | 4.8703 | |

pH 7.1

### Example 8a: Bisabolol 0.06

| Ingredient | Amount in g | | Amount in % |
|---|---|---|---|
| (a) water (3.28 g) + EtOH (0.01 g) | 3.29 | g | 66.97 |
| (b) hyaluronic acid (HA II and HA III 1:1) | 0.07 | g | 1.44 |
| (c) xylitol | 1.5 | g | 30.91 |
| (d) alpha-bisabolol | 0.003 | g | 0.06 |
| (e2) sodium bicarbonate | 0.03 | g | 0.62 |
| Total composition: | 4.893 | g | |

pH 7.1

### Example 8b: Bisabolol 0.01 HA 0.4

| Ingredient | Amount in g | | Amount in % |
|---|---|---|---|
| (a) water (3.24 g) + EtOH (0.01 g) | 3.25 | g | 67.77 |
| (b) hyaluronic acid (HA II and HA III 1:1) | 0.02 | g | 0.42 |
| (c) xylitol | 1.5 | g | 31.28 |
| (d) alpha-bisabolol | 0.0003 | g | 0.01 |
| (e2) sodium bicarbonate | 0.025 | g | 0.52 |
| Total composition: | 4.7953 | g | |

pH 7.0

### Example 8c: 8c Bisabolol 0.01 HA (not According to Invention but Similar to US 2007/0003502 Examples of Table 4

| Ingredient | Amount in g | | Amount in % |
|---|---|---|---|
| (a) water (3.24 g) + EtOH (0.01 g) | 3.25 | g | 67.99 |
| (b) hyaluronic acid (HA Il and HA III 1:1) | 0.005 | g | 0.1 |
| (c) xylitol | 1.5 | g | 31.38 |
| (d) alpha-bisabolol | 0.0003 | g | 0.01 |
| (e2) sodium bicarbonate | 0.025 | g | 0.52 |
| Total composition: | 4.7803 | g | |

pH 6.9

### Example 8d: (not According to Invention) No (d)

| Ingredient | Amount in g | | Amount in % |
|---|---|---|---|
| (a) water (3.26 g) + EtOH (0.01 g) | 3.27 | g | 67.14 |
| (b) hyaluronic acid (HA II and HA III 1:1) | 0.07 | g | 1.44 |
| (c) xylitol | 1.5 | g | 30.8 |
| (d) — | — | | — |
| (e2) sodium bicarbonate | 0.03 | g | 0.62 |
| Total composition: | 4.8703 | g | |

pH 7.1

### Example 9: Similar to Ex 1 of US2020/0390676 (not According to Invention)

| | Amount | Amount |
|---|---|---|
| Ingredient | in g | in % |
| (a) water | 7 | 70 |
| (a) glycerol (glycerin) | 2 | 20 |
| Total (a): | 9 | 90 |
| (b) hyaluronic acid (HA II) | 0.054 | 0.54 |
| (b) carbomer | 0.15 | 1.5 |
| Total (b): | 0.204 | 2.04 |
| (d) eugenol | 0.1 | 1 |
| (c) xylitol | 0.2 | 2 |
| (e) | | |
| (e2) TRIS (CG) | 0.003 | 0.03 |
| (e3) EDTA (CG) | 0.0003 | 0.003 |
| (e5) Parabene (methylparabene) | 0.003 | 0.03 |
| (e15) sodium saccharin (sweetener) | 0.01 | 0.1 |
| (e2) NaOH (50%) | 0.09 | 0.9 |
| (e21) PEG300 (emulsifier) | 0.05 | 0.5 |
| (e21) PVPK90 (emulsifier) | 0.15 | 1.5 |
| (e4) allantoin | 0.01 | 0.1 |
| (e12) dexpanthenol | 0.05 | 0.5 |
| (e9) tocopheryl acetate | 0.05 | 0.5 |
| (e8) sodium ascorbyl phosphate | 0.09 | 0.9 |
| (e21) sodium diphosphate | 0.05 | 0.5 |
| Preservatives (?) | 0 | 0 |
| Total (e) | 0.5563 | 5.53 |
| Flavorant is not defined in the examples. For | | |
| comparison's sake, 1% eugenol (see (d)) was | | |
| used as flavorant (see page 5, first line) | | |
| Total composition: | 10.603 | |

pH 7.5

### Example 9a: Similar to Ex 8 of KR1020000031162 (not According to Invention)

| Ingredient | Amount in g | Amount in % |
|---|---|---|
| (a) water | 5.525 | 55.25 |
| (a) glycerol | 3.0 | 30 |
| (a) propylene glycol | 0.8 | 8 |
| (a) ethanol | 0.4 | 4 |
| Total (a): | 9.725 | 97.25 |
| (b) hyaluronic acid (HA II) | 0.2 | 2 |
| (b) sodium carboxymethyl cellulose | 0.03 | 0.3 |
| Total (b): | 0.23 | 2.3 |
| (c) xylitol | — | — |
| (d) alpha-bisabolol | 0.03 | 0.3 |
| (e15) sodium saccharin | 0.01 | 0.1 |
| (e21) sodium laurylsulfate (SDS) | 0.005 | 0.05 |
| Total (e) | 0.015 | 0.15 |
| Total composition: | 10 g | |

pH 7.5

### Example 9b: Similar to Ex 10 of KR1020000031162 (not According to Invention)

| Ingredient | Amount in g | Amount in % |
|---|---|---|
| (a) water | 7.92 | 82.5 |
| (a) glycerol | 0.2 | 2.2 |
| (a) ethanol | 0.5 | 5 |
| Total (a): | 8.62 | 86.2 |
| (b) hyaluronic acid (HA II) | 0.1 | 1 |
| (c) sorbitol (70%) | 0.7 (?) | 7 (?) |
| (d) alpha-bisabolol | 0.01 | 0.1 |
| (e15) sodium saccharin | 0.02 | 0.2 |
| (e21) PEG 1500 | 0.005 | 0.05 |
| (e1) Cast iron oil (walnut oil) | 0.2 | 2 |
| (e21) sucrose-monolaurate | 0.02 | 0.2 |
| (e21) sodium laurylsulfate (SDS) | 0.005 | 0.05 |
| (e7) menthol | 0.02 | 0.2 |
| 30% further ingredients sorbitol (?) | 0.3 (?) | 3 (?) |
| Total (e) | 0.57 | 2.7 |
| Total composition: | 10 g | |

pH 7.4

### Example 10: Eugenol Comparison to 9

| Ingredient | Amount in g | Amount in % |
|---|---|---|
| (a) water | 7 | 69.6 |
| (a) glycerol | 1.2 | 11.9 |
| Total (a): | 8.2 | 81.5 |
| (b) hyaluronic acid (HA II) | 0.054 | 0.5 |
| (b) carbomer (CG) | 0.15 | 1.5 |
| Total (b): | 0.204 | 2.0 |
| (c) xylitol | 1 | 10 |
| (d) eugenol | 0.1 | 1 |
| (e2) TRIS (CG) | 0.003 | 0.03 |
| (e3) EDTA (CG) | 0.0003 | 0.003 |
| (e5) methylparabene | 0.003 | 0.03 |
| (e15) sodium saccharin | 0.01 | 0.1 |
| (e2) NaOH (50%) | 0.09 | 0.9 |
| (e21) PEG300 | 0.05 | 0.5 |
| (e21) PVPK90 | 015 | 1.5 |
| (e4) allantoin | 0.01 | 0.1 |
| (e12) Dexpanthenol | 0.05 | 0.5 |
| (e8) tocopheryl acetate | 0.05 | 0.5 |
| (e8) sodium ascorbyl phosphate | 0.09 | 0.9 |
| (e21) Sodium diphosphate | 0.05 | 0.5 |
| Flavorant* | | |
| Total (e) | 0.5563 | |
| Total composition: | 10.0603 g | |

*is not defined in the examples of KR1020000031162. For comparison's sake, eugenol (see (d)) was used as flavorant (see page 5, first line of KR1020000031162)
pH 7.2

### Example 10a: Alpha-Bisabolol

| | | |
|---|---|---|
| (a) water | 7 | 70 |
| (a) glycerol | 1.2 | 12 |
| Total (a): | 8.2 | 82 |
| (b) hyaluronic acid (HA II) | 0.054 | 0.5 |
| (b) carbomer | 0.15 | 1.5 |
| Total (b): | 0.204 | 2.0 |
| (c) xylitol | 1 | 10 |
| (d) alpha-bisabolol | 0.1 | 1 |
| (e) TRIS (CG) | 0.003 | 0.03 |
| (e) EDTA (CG) | 0.0003 | 0.003 |
| (e5) methylparabene | 0.003 | 0.03 |
| (e15) sodium saccharin | 0.01 | 0.1 |
| (e2) NaOH (50%) | 0.09 | 0.9 |
| (e21) PEG300 | 0.05 | 0.5 |
| (e21) PVPK90 | 0.15 | 1.5 |
| (e4) allantoin | 0.01 | 0.1 |
| (e12) dexpanthenol | 0.05 | 0.5 |
| (e8) tocopheryl acetate | 0.05 | 0.5 |
| (e8) sodium ascorbyl phosphate | 0.09 | 0.9 |
| (e21) sodium diphosphate | 0.05 | 0.5 |
| Total (e) | 0.5563 | |
| Total composition: | 10.0603 g | |

pH 7.3

### Example 10b: Curcumin

| | | |
|---|---|---|
| (a) water | 7 | 70 |
| (a) glycerol | 1.2 | 12 |
| Total (a): | 8.2 | 82 |
| (b) hyaluronic acid (HA II) | 0.054 | 0.5 |
| (b) carbomer | 0.15 | 1.5 |
| Total (b): | 0.204 | 2.0 |
| (c) xylitol | 1 | 10 |
| curcumin | 0.1 | 1 |
| (e2) TRIS (CG) | 0.003 | |
| (e) EDTA (CG) | 0.0003 | |
| (e5) methylparabene | 0.003 | |
| (e15) sodium saccharin | 0.01 | |
| (e2) NaOH (50%) | 0.09 | |
| (e21) PEG300 | 0.05 | |
| (e21) PVPK90 | 0.15 | |
| (e4) allantoin | 0.01 | |
| (e12) dexpanthenol | 0.05 | |
| (e8) tocopheryl acetate | 0.05 | |
| (e8) sodium ascorbyl phosphate | 0.09 | |
| (e21) sodium diphosphate | 0.05 | |
| Total (e) | 0.5563 | |
| Total composition: | 10.0603 g | |

pH 7.3

### Example 10c: Beta-Caryophyllene

| | | |
|---|---|---|
| (a) water | 7 | 70 |
| (a) glycerol | 1.2 | 12 |
| Total (a): | 8.2 | 82 |
| (b) hyaluronic acid (HA II) | 0.054 | 0.5 |
| (b) carbomer | 0.15 | 1.5 |
| Total (b): | 0.204 | 2.0 |
| (c) xylitol | 1 | 10 |
| Beta-caryophyllene | 0.1 | 1 |
| (e2) TRIS (CG) | 0.003 | |
| (e) EDTA (CG) | 0.0003 | |
| (e5) methylparabene | 0.003 | |
| (e15) sodium saccharin | 0.01 | |
| (e2) NaOH (50%) | 0.09 | |
| (e21) PEG300 | 0.05 | |
| (e21) PVPK90 | 0.15 | |
| (e4) allantoin | 0.01 | |
| (e12) dexpanthenol | 0.05 | |
| (e8) tocopheryl acetate | 0.05 | |
| (e8) sodium ascorbyl phosphate | 0.09 | |
| (e21) sodium diphosphate | 0.05 | |
| Total (e) | 0.5563 | |
| Total composition: | 10.603 g | |

pH 7.2

### Example 11: Chamomile CE II+Mentha

| | Amount | Amount |
|---|---|---|
| Ingredient | in g | in % |
| (a) water | 3.50 | |
| (a) water (PRO) | 0.19 | |
| (a) ethanol (PRO) | 0.08 | |
| (a) Pentylene glycol (also moisturizer) | 0.18 | |
| (a) glycerol (also moisturizer) | 0.09 | |
| Total (a): | 4.04 | 67.94 |
| (b) hyaluronic acid (HA III) | 0.07 | 1.18 |
| (b) D-Galakto-D-Mannan | 0.05 | 0.84 |
| Total (b): | 0.12 | 2.02 |
| (c) Xylitol | 1.5 | 25.23 |
| (d) matricin (CE II) | 0.0002 | |
| (d) alpha-bisabolol (CE II) | 0.00056 | |
| Total (d): | 0.00076 | 0.01 |
| (e1) lotus extract (lyophilisate) | 0.05 | |
| (e4) catechin | 0.09 | |
| (e1) further ingredients (catechin) | 0.01 | |
| (e1) further ingredients (CE II) | 0.03924 | |
| (e2) sodium bicarbonate | 0.01 | |
| (e1) PRO | 0.0024 | |
| (e21) glyceryl caprylate | 0.08 | |
| (e1) Mentha arvensis leaf oil (menthol content 63%) | 0.002 | |
| (e4) limonene | 0.001 | |
| (e7) linalool (flavoring) | 0.001 | |
| Total (e): | 0.28564 | 4.8 |
| Total composition: | 5.9464 | |

pH 7.1

### Example 11a: (CHX)

| | Amount | Amount |
|---|---|---|
| Ingredient | in g | in % |
| (a) water | 3.54 | |
| (a) water (PRO) | 0.19 | |
| (a) ethanol (PRO) | 0.08 | |
| (a) Pentylene glycol (also moisturizer) | 0.18 | |
| (a) glycerol (also moisturizer) | 0.09 | |
| Total (a): | 4.08 | 67.94 |
| (b) hyaluronic acid (HA III) | 0.07 | |
| (b) D-Galakto-D-Mannan | 0.05 | |
| Total (b): | 0.12 | 2.02 |
| (c) Xylitol | 1.5 | 25.23 |
| (d) chlorhexidine | 0.00076 | 0.01 |
| (e1) lotus extract (lyophilisate) | 0.05 | |
| (e4) catechin | 0.09 | |
| (e1) further ingredients (catechin) | 0.01 | |
| (e2) sodium bicarbonate | 0.01 | |
| (e1) PRO | 0.0024 | |
| (e21) glyceryl caprylate | 0.08 | |
| (e1) Mentha arvensis leaf oil (menthol content 63%) | 0.002 | |
| (e4) limonene | 0.001 | |
| (e7) linalool (flavoring) | 0.001 | |
| Total (e): | 0.2464 | 4.8 |
| Total composition: | 5.9471 | |

pH 7.0

### Example 11b: (Cetylpyridinium Chloride)

| | Amount | Amount |
|---|---|---|
| Ingredient | in g | in % |
| (a) water | 3.54 | |
| (a) water (PRO) | 0.19 | |
| (a) ethanol (PRO) | 0.08 | |
| (a) Pentylene glycol (also moisturizer) | 0.18 | |
| (a) glycerol (also moisturizer) | 0.09 | |
| Total (a): | 4.08 | 67.94 |
| (b) hyaluronic acid (HA III) | 0.07 | |
| (b) D-Galakto-D-Mannan | 0.05 | |
| Total (b): | 0.12 | 2.02 |
| (c) Xylitol | 1.5 | 25.23 |
| (d) hexadecylpyridinium chloride monohydrate | 0.00076 | 0.01 |
| (e1) lotus extract (lyophilisate) | 0.05 | |
| (e4) catechin | 0.09 | 0.02 |
| (e1) further ingredients (catechin) | 0.01 | |
| (e2) sodium bicarbonate | 0.01 | |
| (e1) PRO | 0.0024 | 0.17 |
| (e21) glyceryl caprylate | 0.08 | |
| (e1) Mentha arvensis leaf oil (menthol content 63%) | 0.002 | |
| (e4) limonene | 0.001 | 0.03 |
| (e7) linalool (flavoring) | 0.001 | |
| Total (e): | 0.2464 | 4.8 |
| Total composition: | 5.9471 | |

pH 7.0

### Example 12: CE II Menthol PBS

| | Amount | Amount |
|---|---|---|
| Ingredient | in g | in % |
| (a) water (incl. water from 1xPBS) | 3.69 | |
| (a) ethanol | 0.08 | |
| (a) pentylene glycol (moisturizer) | 0.18 | |
| (a) glycerol (moisturizer) | 0.09 | |
| Total (a): | 4.04 | 66.48 |
| (b) hyaluronic acid (HA III) | 0.08 | |
| (b) D-Galakto-D-Mannan | 0.08 | |
| Total (b): | 0.16 | 2.63 |
| (c) Xylitol | 1.5 | 24.68 |
| (d) matricin (CE II) | 0.0002 | |
| (d) alpha-bisabolol (CE II) | 0.0006 | |
| Total (d): | 0.0008 | 0.01 |
| (e1) lotus extract (lyophilisate) | 0.05 | |
| (e4) EGCG | 0.14 | |
| (e1) further ingredients (CE II) | 0.042 | |
| (e2)/(e22) 1xPBS (0.25 ml) NaCl Na₂HPO₄ | 0.01 | |
| and KH₂PO₄ | | |
| (e1) PRO | 0.0026 | |
| (e21) glyceryl caprylate | 0.08 | |
| (e18) menthol | 0.05 | |
| Total (e): | 0.002 | |
| | 0.3766 | 6.2 |
| Total composition: | 6.0774 | |

pH 7.1

### Example 13: Catechin+Cellulose+Vitamins+Colorant+Fluoride

| | Amount | Amount |
|---|---|---|
| Ingredient | in g | in % |
| (a) water | 3.50 | |
| (a) pentylene glycol (also humectant) | 0.18 | |
| (a) glycerol (also humectant) | 0.09 | |
| Total (a): | 3.77 | 63.95 |
| (b) hyaluronic acid (HA II) | 0.08 | |
| (b) hydroxypropyl cellulose (CAS 9004-64-2) | 0.08 | |
| Total (b): | 0.16 | 2.71 |
| (c) Xylitol | 1.5 | 25.45 |
| (d) matricin (CE II) | 0.0002 | |
| (d) alpha-bisabolol (CE II) | 0.0006 | |
| Total (d): | 0.0008 | 0.01 |
| (e1) lotus extract (lyophilisate) | 0.05 | |
| (e4) EGCG/EC (6:1) | 0.14 | |
| (e1) further ingredients (CE II) | 0.042 | |
| (e21) glyceryl caprylate | 0.08 | |
| (e8) vitamin A (retinol) | 0.025 | |
| (e8) vitamin C (sodium ascorbyl phosphate) | 0.025 | |
| (e8) vitamin E (tocopheryl acetate) | 0.025 | |
| (e8) vitamin B (B9 folic acid) | 0.025 | |
| (e16) beta-carotene | 0.001 | |
| (e14) stanous fluoride | 0.001 | |
| (e18) N-Ethyl-p-menthane-3-carboxamide | 0.05 | |
| Total (e): | 0.464 | 7.87 |
| Total composition: | 5.8948 | |

pH 6.8

### Example 13a: Cellulose+Vitamins+Colorant+Fluoride

| | Amount | Amount |
|---|---|---|
| Ingredient | in g | in % |
| (a) water | 3.50 | |
| (a) pentylene glycol (also humectant) | 0.18 | |
| (a) glycerol (also humectant) | 0.09 | |
| Total (a): | 3.77 | 63.95 |
| (b) hyaluronic acid (HA II) | 0.08 | |
| (b) sodium carboxymethyl cellulose | 0.08 | |
| (CAS 9004-32-4) | | |
| Total (b): | 0.16 | 2.71 |
| (c) Xylitol | 1.5 | 25.45 |
| (d) matricin (CE II) | 0.0002 | |
| (d) alpha-bisabolol (CE II) | 0.0006 | |
| Total (d): | 0.0008 | 0.01 |
| (e1) lotus extract (lyophilisate) | 0.05 | |
| (e4) EGCG/EC (6:1) | 0.14 | |
| (e1) further ingredients (CE II) | 0.042 | |
| (e21) glyceryl caprylate | 0.08 | |
| (e8) vitamin A (retinol) | 0.025 | |
| (e8) vitamin C (sodium ascorbyl phosphate) | 0.025 | |
| (e8) vitamin E(tocopheryl acetate) | 0.025 | |
| (e8) vitamin B (B9 folic acid) | 0.025 | |
| (e16) beta-carotene | 0.001 | |
| (e14) stanous fluoride | 0.001 | |
| (e18) N-Ethyl-p-menthane-3-carboxamide | 0.05 | |
| Total (e): | 0.464 | 7.87 |
| Total composition: | 5.8948 | |

pH 6.9

### Example 14: Improving the Mouthfeel of Aligner Splint Supports

At the beginning, the users were informed about the area of application. The users were instructed how oral hygiene should be carried out during the study period (brushing their teeth twice a day for 2 min in the morning and in the evening). This oral hygiene was started one week before the start of the experiment in order to enable comparable conditions. All participants had comparable prerequisites before the start of oral hygiene. During the test, one application of 0.5 ml each was placed on the left, right and center of the inside of the aligner splints in the morning, at noon and in the evening after meals and the aligner splints were (re)introduced into the oral cavity.

Half of the users were female and half male, all users were between 30 (thirty) and 60 (sixty) years old, the samples were randomly assigned, but care was taken to ensure that per sample 5 (five) male users and 5 (five) female users received said sample. A questionnaire was included in the first evaluation. The interplay of mouth taste, bad breath and dry mouth was queried. These factors are queried using a Quality of Life questionnaire in the form of a journal. The users were asked to rate their taste in mouth, bad breath and dry mouth on a scale from 1 to 5. In order to avoid misunderstandings, it was explicitly pointed out in advance that for mouth taste 1 was defined as pleasant (most positive) and 5 as unpleasant (most negative), for bad breath 1 was defined as no bad breath perceived (most positive) and 5 as strong bad breath perceived (most negative) (bad breath should be determined by exhaling into your hand and immediately breathing in through your nose), and for dry mouth 1 was defined as no feeling of dryness (most positive) and 5 as extremely dry mouth (most negative).

All aligner splint users had an unremarkable oral appearance prior to aligner therapy. All test results were collected once a week for six weeks on the first day of each week and at the end of the sixth week. The aligner splints were worn overnight for the 9-hour query. The one hour query took place one hour after lunch and after putting on the aligner again.

Each sample was given to 10 (ten) different aligner splint users. The experiment was carried out as a double-blind experiment. Neither the users nor the attending dentist knew which sample number was assigned to which sample. Samples 1 to 3 corresponded to commercial products, sample 4 was a placebo (water) and samples 5 to 11 correspond to examples 1 to 7.

The mean value was calculated from the seven answer questionnaires per user. For non-natural numbers and if the digit at the first decimal place was a 0, 1, 2, 3 or 4, then it was rounded down. If the digit at the first remaining decimal place was a 5, 6, 7, 8 or 9, the number was rounded up. Each value is therefore based on the average of 70 individual values (seven questionnaires per user with ten users per sample). In twelve cases it happened that one day was not entered in the corresponding questionnaire. In these cases, the evaluation was calculated with 69 instead of 70 individual values (see Table 1). There was no overlap in forgotten entries, i.e. there were 69 or 70 individual values for all queries. All tests with the users were performed under a non-disclosure agreement.

TABLE 1
Results of the questionairs

| | Mouth | | Mouth | | Bad | | Mouth | |
|---|---|---|---|---|---|---|---|---|
| | taste | | taste | | breath | | dryness | |
| Group | (1 h) | | (8 h) | | (8 h) | | (8 h) | |
| Oral Gel (Kamistad, | 1 | (2) | 1 | | 1 | (0) | 1 | (—) |
| STADA: lidocaine | 2 | (3) | 2 | (1) | 2 | (1) | 2 | (—) |
| hydrochloride and | 3 | (3) | 3 | (2) | 3 | (1) | 3 | (1) |
| chamomile flower | 4 | (2) | 4 | (5) | 4 | (5) | 4 | (3) |
| extract | 5 | (0) | 5 | (2) | 5 | (3) | 5 | (6) |
| Mouth water (Kamistad, | 1 | (1) | 1 | (—) | 1 | (—) | 1 | (—) |
| STADA, chlorhexidin | 2 | (2) | 2 | (—) | 2 | (—) | 2 | (1) |
| (0.1%), chamomile | 3 | (2) | 3 | (1) | 3 | (—) | 3 | (—) |
| flower extract, | 4 | (4) | 4 | (4) | 4 | (6) | 4 | (2) |
| hyaluronic acid) | 5 | (1) | 5 | (5) | 5 | (4) | 5 | (7) |
| Mouth creme (MI Paste | 1 | (3) | 1 | (—) | 1 | (—) | 1 | (1) |
| Plus Mint, GC Germany | 2 | (2) | 2 | (1) | 2 | (1) | 2 | (1) |
| GmbH) | 3 | (3) | 3 | (4) | 3 | (2) | 3 | (2) |
| | 4 | (2) | 4 | (4) | 4 | (5) | 4 | (4) |
| | 5 | (—) | 5 | (1) | 5 | (2) | 5 | (2) |
| Control (Placebo) | 1 | (—) | 1 | (—) | 1 | (—) | 1 | (—) |
| | 2 | (3) | 2 | (—) | 2 | (—) | 2 | (—) |
| | 3 | (4) | 3 | (1) | 3 | (1) | 3 | (—) |
| | 4 | (3) | 4 | (3) | 4 | (2) | 4 | (1) |
| | 5 | (—) | 5 | (6) | 5 | (7) | 5 | (9) |
| Example 1 | 1 | (3) | 1 | (—) | 1 | (2) | 1 | (1) |
| | 2 | (3) | 2 | (4) | 2 | (4) | 2 | (4) |
| | 3 | (4) | 3 | (5) | 3 | (3) | 3 | (4) |
| | 4 | (—) | 4 | (1) | 4 | (1) | 4 | (1) |
| | 5 | (—) | 5 | (—) | 5 | (—) | 5 | (—) |
| Example 2 | 1 | (1) | 1 | (1) | 1 | (1) | 1 | (3) |
| | 2 | (4) | 2 | (4) | 2 | (6) | 2 | (4) |
| | 3 | (4) | 3 | (5) | 3 | (3) | 3 | (3) |
| | 4 | (1) | 4 | (—) | 4 | (—) | 4 | (—) |
| | 5 | (—) | 5 | (—) | 5 | (—) | 5 | (—) |
| Example 3 (not | 1 | (1) | 1 | (—) | 1 | (—) | 1 | (—) |
| according to invention) | 2 | (3) | 2 | (1) | 2 | (—) | 2 | (1) |
| | 3 | (5) | 3 | (3) | 3 | (2) | 3 | (3) |
| | 4 | (1) | 4 | (5) | 4 | (5) | 4 | (5) |
| | 5 | (—) | 5 | (—) | 5 | (4) | 5 | (1) |
| Example 4 | 1 | (3) | 1 | (—) | 1 | (1) | 1 | (1) |
| | 2 | (4) | 2 | (2) | 2 | (4) | 2 | (3) |
| | 3 | (2) | 3 | (6) | 3 | (3) | 3 | (5) |
| | 4 | (1) | 4 | (2) | 4 | (2) | 4 | (1) |
| | 5 | (—) | 5 | (1) | 5 | (—) | 5 | (—) |
| Example 5 | 1 | (2) | 1 | (—) | 1 | (—) | 1 | (1) |
| | 2 | (5) | 2 | (2) | 2 | (5) | 2 | (3) |
| | 3 | (2) | 3 | (5) | 3 | (3) | 3 | (4) |
| | 4 | (1) | 4 | (3) | 4 | (2) | 4 | (2) |
| | 5 | (—) | 5 | (1) | 5 | (—) | 5 | (—) |
| Example 6 (not | 1 | (2) | 1 | (—) | 1 | (—) | 1 | (—) |
| according to invention) | 2 | (3) | 2 | (—) | 2 | (—) | 2 | (—) |
| | 3 | (4) | 3 | (5) | 3 | (2) | 3 | (3) |
| | 4 | (1) | 4 | (4) | 4 | (5) | 4 | (5) |
| | 5 | (—) | 5 | (1) | 5 | (3) | 5 | (2) |
| Example 7 | 1 | (—) | 1 | (—) | 1 | (1) | 1 | (3) |
| | 2 | (4) | 2 | (2) | 2 | (5) | 2 | (3) |
| | 3 | (5) | 3 | (4) | 3 | (4) | 3 | (4) |
| | 4 | (1) | 4 | (3) | 4 | (—) | 4 | (—) |
| | 5 | (—) | 5 | (1) | 5 | (—) | 5 | (—) |

### Example 15: Reduction of the Bleeding on Probing Index (BOP)

BOP is a diagnostic procedure for assessing inflammation of the gingiva or periodontium. The dentist used a blunt periodontal probe (WHO probe) to probe the bottom of the gingival sulcus at six measuring points per tooth under low pressure (the examined teeth were of course part of the aligner splint therapy). Bleeding spots were documented, and the percentage of bleeding was calculated after a complete examination. The lower the number of bleedings, the more stable the gum is. A value of 25% applies as a threshold value. All users were held to brush their teeth two times a day after breakfast and dinner during the test period. During the test, one application of 0.5 ml each was placed on the left, right and the center of the inside of the aligner splints in the morning, at noon and in the evening after meals and the aligner splints were (re)introduced into the oral cavity. The control I (placebo) was water. All applications were carried out with a 1 ml syringe. If not stated otherwise, the results are based on groups of ten individuals (see DE10 2022 003 105.1, filing date Aug. 26, 2023). For Example 1 and 7, five further individuals were treated in addition to the first ten individuals (results for Example 1 of the first ten individuals were 2/10 at day one, 3/10 after six weeks, 2/10 after twelve weeks; results for Example 7 of the first ten individuals were 1/10 at day one, 1/10 after six weeks and 2/10 after twelve weeks (see DE10 2022 003 105.1)).

Example 13 and Control II were performed with 5 individuals per group. The individuals in this group were no aligner splint users but individuals with bleeding gum problems over at least two months before the test period. The individuals provided 1.5 ml of Example 13 via a toothbrush in the morning and evening after the regular teeth brushing. The Control placebo II was provided as 1.5 ml of an 0.001% menthol in 1.5% hydroxypropyl cellulose hydrogel (1.5 ml per application in the morning and evening after the teeth brushing).

The data in table 2 give the number of users whose BOP index was above 25%. All tests were performed as double-blind tests, i.e., a third person labeled the opaque sample bottles and neither the individual nor the dentist knew which sample number referred to mouth water, mouth creme, control or any of the examples (all tests with the individuals were performed under non-disclosure agreements).

TABLE 2
Results of the BOP tests

| | BOP Index | BOP Index | BOP Index |
|---|---|---|---|
| | higher 25% | higher 25% | Higher 25% |
| group | day 1 | after 6 weeks | after 12 weeks |
| Oral Gel (Kamistad) | 1 | 4 | 4 |
| Mouth water (Kamistad) | 2 | 5 | 7 |
| Mouth creme (MI Paste | 1 | 5 | 7 |
| plus, GC) | | | |
| Control (Placebo I) | 2 | 7 | 6 |
| Example 1¹ | 4 | 4 | 3 |
| Example 1a¹ | 4 | 2 | 1 |
| Example 2 | 1 | 1 | 1 |
| Example 3 (not | 2 | 5 | 6 |
| according to invention) | | | |
| Example 4 | 2 | 3 | 1 |
| Example 5 | 0 | 2 | 2 |
| Example 6 (not | 1 | 4 | 6 |
| according to invention) | | | |
| Example 7¹ | 3 | 2 | 2 |
| Example 7a¹ | 3 | 2 | 0 |

| | BOP Index | BOP Index | BOP Index |
|---|---|---|---|
| | higher 25% | higher 25% | Higher 25% |
| | day 1 | after 2 weeks | after 4 weeks |
| Example 13² | 5/5 | 3/5 | 2/5 |
| Control (Placebo II)² | 5/5 | 4/5 | 5/5 |

¹basis were fifteen individuals
²basis were five individuals without aligner splints but with a general high BOP.

Especially the placebo I group and the mouth water group complained about a problematic application. In addition, the placebo, the mouth wash, and the mouth creme as well as the groups testing Example 3 and Example 6 (both not according to the invention) showed a remarkable deterioration of the gum status during the test phase. The placebo, mouth wash, mouth creme and Example 6 users described a fast dissolving of the firstly strong taste of the application indicating the applications do not stick long enough to the gum and teeth surface. The reduced amount of xylitol in Example 3 may not inhibit the plaque formation strong enough. These detrimental effects were not that severe in the group of oral gel users (Kamistad) but still present indicating the gel shows a kind of longer lasting effect.

In contrast, examples 1, 1a, 2, 4, 5, 7, 7a showed very good results, even after 12 weeks. Moreover, when comparing examples 1 with 1a and 7 with 7a, respectively, the presence of an antioxidant (sample 1a and 7a (in both cases catechins from tea) results in a better protection of the gum or even a regeneration of gum.

### Example 16: Gum Recession Due to Wearing Aligners

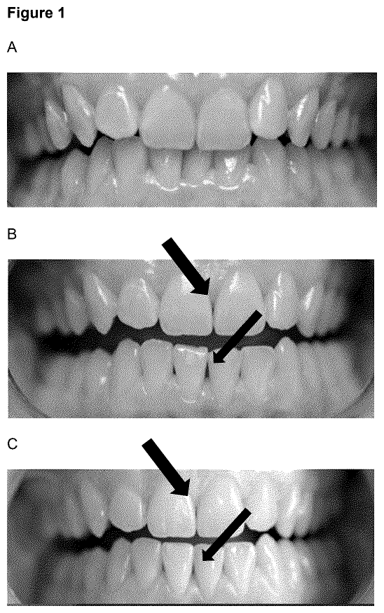

The dentist regularly examines the health of the gum, thereby also observing the height of the gum in the interdental spaces. The gum of aligner splint users retracts due to stress while wearing the aligner splint. FIG. 1 shows an example of a set of teeth with healthy gum before the start of an aligner therapy (1A), the same set of teeth twelve months after the start of aligner splint therapy with gum recession and inflammatory (arrow) (1B) and the same set of teeth seven days after application of Example 2 according to the invention (and seven days after the measurement for image 1B). FIG. 1 can also be provided as colored picture. Due to the long-time frames and difficult comparability, the focus was shifted to the other experiments demonstrating the superior effect of the compositions of the invention over the prior art.

### Example 17: Hypersensitivity

Sensitivity was tested with a thermal stimulus. This involved blowing air from a dental air blower over the tooth. The room temperature was 21° C., that of the teeth is 33° on average. The colder air extracts heat from the tooth surface due to the temperature difference. The air flow stimulus was applied to the tooth at a distance of 1 cm for a duration of 1 s at a temperature of 21° C. and a pressure of 4.14 bar.

The evaluation was carried out using the Schiff Cold Air Sensitivity Scale (SCA) and noted by the dentist:
0: subject does not respond to airflow stimulus;
1: Subject responds to airflow stimulus but does not request removal of stimulus;
2: subject responds to airflow stimulus and requests removal of stimulus or moves away from stimulus;
3: Subject responds to the airflow stimulus, finds it painful, and requests removal of the stimulus.

First results show that in five examined individuals (1-5) with hypersensitivity, the latter starts to decrease after 7 days with three treatments (1.5 ml of a composition according to Example 29. Further individuals with hypersensitivity were treated with compositions according to examples 6 (not according to invention) 7, 7a, 11 and 12, respectively, as well as with placebo (0.001% menthol in 1.5% hydroxypropyl cellulose hydrogel (Gel) (C1 and 02)) (see Table 3). Per application, 1.5 ml of the respective compositions were applied to the teeth and gum in the morning, at noon and in the evening by gentle rub in with a toothbrush for one minute. All tests were performed as double-blind tests, i.e., a third person labeled the opaque sample bottles and neither the individual nor the dentist knew which sample number referred to which sample (all tests with the individuals were performed under non-disclosure agreements):

TABLE 3
Results of the SCA tests

| | | SCA prior | SCA after | SCA after | SCA after |
|---|---|---|---|---|---|
| subject | Sample | to therapy | 7 days | 14 days | 21 days |
| 1 | Ex 2 | 3 | 2 | 1 | 1 |
| 2 | Ex 2 | 3 | 3 | 2 | 1 |
| 3 | Ex 2 | 3 | 2 | 0 | 0 |
| 4 | Ex 2 | 3 | 2 | 2 | 1 |
| 5 | Ex 2 | 3 | 3 | 2 | 0 |
| 6 | Ex 2 | 3 | 2 | 1 | 1 |
| C1 | Gel | 3 | 3 | 3 | 2 |
| C2 | Gel | 3 | 2 | 3 | 3 |
| 9 | Ex 6 | 3 | 3 | 2 | 2 |
| 10 | Ex 6 | 3 | 2 | 3 | 2 |
| 11 | Ex 6 | 3 | 2 | 2 | 2 |
| 12 | Ex 6 | 3 | 3 | 2 | 3 |
| 13 | Ex 7 | 3 | 2 | 2 | 0 |
| 14 | Ex 7 | 3 | 2 | 1 | 1 |
| 15 | Ex 7 | 3 | 3 | 2 | 1 |
| 16 | Ex 7 | 3 | 2 | 2 | 2 |
| 17 | Ex 7 | 3 | 3 | 2 | 0 |
| 18 | Ex 7 | 3 | 3 | 1 | 0 |
| 21 | Ex 7a | 3 | 2 | 0 | 1 |
| 22 | Ex 7a | 3 | 1 | 1 | 1 |
| 23 | Ex 7a | 3 | 2 | 1 | 0 |
| 24 | Ex 7a | 3 | 1 | 1 | 1 |
| 25 | Ex 7a | 3 | 1 | 0 | 0 |
| 26 | Ex 7a | 3 | 3 | 1 | 0 |
| 29 | Ex 11 | 3 | 2 | 1 | 0 |
| 30 | Ex 11 | 3 | 1 | 1 | 1 |
| 31 | Ex 11 | 3 | 2 | 1 | 0 |
| 32 | Ex 11 | 3 | 2 | 0 | 0 |
| 33 | Ex 12 | 3 | 1 | 0 | 1 |
| 34 | Ex 12 | 3 | 2 | 1 | 0 |
| 35 | Ex 12 | 3 | 1 | 1 | 0 |
| 36 | Ex 12 | 3 | 2 | 1 | 1 |

The control group C1 and 02 as well as individuals with a composition according to example 6 showed no significant change in hypersensitivity. In contrast, compositions according to the invention showed a significant reduction of hypersensitivity already after 7 days, with very good results after 21 days of daily treatment. The presence of antioxidants (see examples 7a, 11 and 12 (with antioxidant) vs. examples 2 and 7) seem to accelerate this beneficial effect.

### Example 18: Determination of Viscosity

The kinematic viscosity was determined using a rotary rheometer from Bohlin Instruments (Gemini—Advanced Rheometer). A cone plate with a standard smooth surface (stainless steel) of 4°/40 mm diameter (order no 3628/J01/41 from Bohlin instruments, Pfortsheim, Germany) served as the measurement geometry. All measurements were performed in triplicate. Measurements of examples 2, 4 and 5 with a Kinexus Prime pro+ Rheometer with a standard smooth surface (stainless steel) of 4°/40 mm (order no KNX(2036 from NETZSCH-Geratebau GmbH, Selm, Germany) confirmed the results with examples 2, 4 and 5.

The measurement for example 2 is exemplarily listed in table 4 (made with Gemini—Advanced Rheometer). Table 5 summarizes the further measurements.

TABLE 4
results of rheometric viscosity measurement od Example 2

| | | shear | shear | | target- |
|---|---|---|---|---|---|
| Time | temperature | stress | rate | viscosity | shear rate |
| [s] | [° C.] | [Pa] | [1/s] | [Pas] | [1/s] |
| 51.89 | 21.9 | 30.01 | 0.184 | 163.1 | 0.1823 |
| 78.94 | 21.9 | 42.91 | 0.3649 | 117.6 | 0.3674 |
| 93.66 | 22 | 60.28 | 0.7365 | 81.84 | 0.7405 |
| 102.3 | 21.9 | 81.23 | 1.468 | 55.32 | 1.492 |
| 108.2 | 21.9 | 102.5 | 2.753 | 37.26 | 2.721 |
| 112.8 | 21.9 | 122.3 | 4.531 | 27 | 4.488 |
| 116.6 | 21.9 | 138.9 | 7.316 | 18.99 | 7.404 |
| 119.9 | 21.9 | 157 | 12.11 | 12.96 | 12.21 |
| 123 | 21.9 | 167.6 | 16.44 | 10.19 | 16.49 |
| 126 | 21.9 | 172.3 | 20.07 | 8.587 | 20.15 |
| 128.9 | 21.9 | 178 | 24.54 | 7.255 | 24.61 |
| 131.8 | 21.9 | 186.6 | 33.17 | 5.625 | 33.23 |
| 134.5 | 21.9 | 201 | 54.74 | 3.672 | 54.82 |
| 137.2 | 21.9 | 210.9 | 81.5 | 2.588 | 81.82 |
| 139.8 | 21.9 | 214.9 | 99.53 | 2.159 | 99.96 |
| 142.5 | 21.9 | 220.2 | 121.5 | 1.813 | 122.1 |
| 145.1 | 21.9 | 225.9 | 148.4 | 1.523 | 149.2 |
| 147.7 | 21.9 | 240.8 | 221.3 | 1.088 | 222.7 |
| 150.3 | 21.9 | 256.1 | 330.2 | 0.7754 | 332.3 |
| 152.9 | 21.9 | 260.8 | 403.7 | 0.646 | 406 |
| 155.5 | 21.9 | 270.3 | 492.5 | 0.5488 | 495.9 |
| 158 | 21.9 | 271.1 | 545.4 | 0.4972 | 548.2 |
| 160.7 | 21.9 | 273.1 | 602.8 | 0.453 | 605.9 |
| 163.2 | 21.9 | 278.1 | 665.9 | 0.4177 | 669.7 |
| 165.8 | 21.9 | 282.2 | 736.3 | 0.3832 | 740.2 |
| 168.4 | 21.9 | 285.4 | 813.5 | 0.3508 | 818.1 |
| 171 | 21.9 | 284.8 | 899 | 0.3168 | 904.3 |
| 173.6 | 21.9 | 295.4 | 993.8 | 0.2973 | 999.4 |
| 176.1 | 21.9 | 313.4 | 1099 | 0.2853 | 1105 |
| 178.7 | 21.9 | 265.9 | 1214 | 0.219 | 1221 |
| 181.3 | 21.9 | 209 | 1101 | 0.1898 | 1105 |
| 183.9 | 21.9 | 218 | 995.8 | 0.2189 | 999.4 |
| 186.5 | 21.9 | 225 | 901.5 | 0.2496 | 904.3 |
| 189 | 21.9 | 195.5 | 815.7 | 0.2396 | 818.1 |
| 191.6 | 21.9 | 199.6 | 737.6 | 0.2706 | 740.2 |
| 194.2 | 21.9 | 193 | 667.6 | 0.2891 | 669.7 |
| 196.8 | 21.8 | 188.3 | 603.7 | 0.3118 | 605.9 |
| 199.4 | 21.9 | 184.5 | 546.1 | 0.3378 | 548.2 |
| 202 | 21.9 | 182.3 | 494.4 | 0.3687 | 495.9 |
| 204.5 | 21.9 | 177.7 | 405.2 | 0.4385 | 406 |
| 207.2 | 21.9 | 176.4 | 331.2 | 0.5325 | 332.3 |
| 209.8 | 21.8 | 172 | 222.4 | 0.7734 | 222.7 |
| 212.4 | 21.9 | 176.6 | 149 | 1.185 | 149.2 |
| 215 | 21.9 | 184.6 | 121.6 | 1.518 | 122.1 |
| 217.7 | 21.9 | 184.2 | 99.53 | 1.851 | 99.96 |
| 220.4 | 21.8 | 181.6 | 81.46 | 2.229 | 81.82 |
| 223.1 | 21.8 | 172.1 | 54.49 | 3.158 | 54.82 |
| 225.9 | 21.8 | 160.3 | 32.94 | 4.867 | 33.23 |
| 228.9 | 21.8 | 155 | 24.44 | 6.34 | 24.61 |
| 231.9 | 21.8 | 151.6 | 20.04 | 7.565 | 20.15 |
| 235 | 21.8 | 147.4 | 16.41 | 8.981 | 16.49 |
| 238.3 | 21.8 | 139.9 | 12.19 | 11.48 | 12.21 |
| 242.1 | 21.8 | 126.4 | 7.42 | 17.03 | 7.404 |
| 246.6 | 21.8 | 111.3 | 4.433 | 25.1 | 4.488 |
| 252.5 | 21.8 | 95 | 2.67 | 35.58 | 2.721 |
| 261.1 | 21.8 | 77.52 | 1.499 | 51.71 | 1.492 |
| 275.9 | 21.8 | 57.19 | 0.738 | 77.49 | 0.7405 |
| 303 | 21.8 | 40.21 | 0.3662 | 109.8 | 0.3674 |
| 354.9 | 21.8 | 26.96 | 0.183 | 147.4 | 0.1823 |

TABLE 5
Rheometric viscosity data

| Example | Viscosity at 1 [1/s] in [Pas] | Yield point [Pa] |
|---|---|---|
| 1 | 23 ± 6 | Between 1 and 4 |
| 2 | 60 ± 4 | 20 ± 2 |
| 3 | 58 ± 3 | 18 ± 2 |
| 4 | 48 ± 3 | 15 ± 2 |
| 5 | 45 ± 4 | 16 ± 3 |
| 6 | Newtonian fluid | Newtonian fluid |
| 7 | 58 ± 3 | 24 ± 4 |
| 8b | 23 ± 3 | 12 ± 2 |
| 8c | Newtonian fluid | Newtonian fluid |
| 9 | 69 ± 3 | 23 ± 4 |
| 9a | 60 ± 3 | 19 ± 3 |
| 10 | 75 ± 4 | 26 ± 4 |
| 11 | 65 ± 6 | 25 ± 3 |

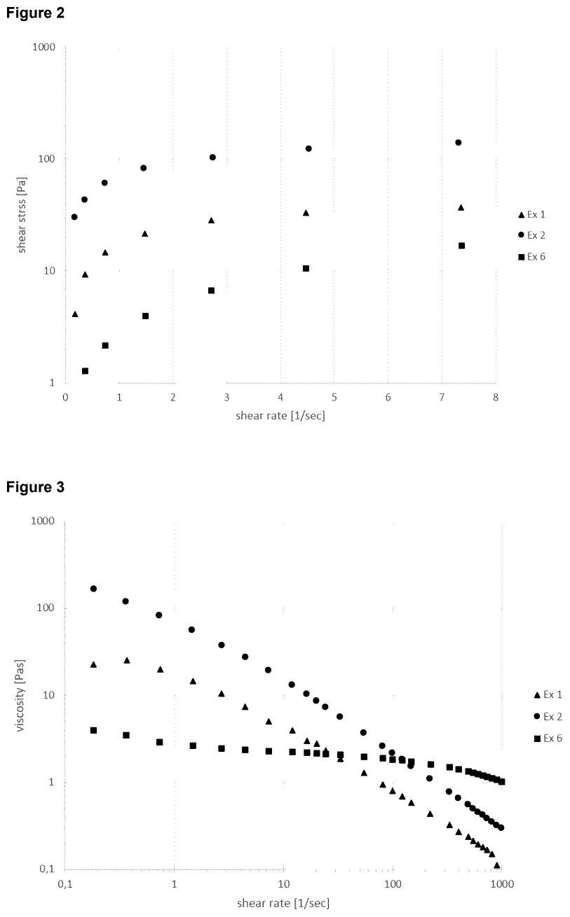

The viscosity value at a shear rate of 1 [1/s] and the extrapolated flow point at a shear rate of 0 [1/s] were used as characterization values. Exemplarily, FIG. 2 shows the shear stress vs. the shear rate and FIG. 3 shows the viscosity as viscosity vs. shear rate for examples 1, 2 and 6 (not according to the invention). In Example 2, the gel structure was built correctly, with shear thinning leading to good applicability. The viscosity at a shear rate of 1 [1/s] in the composition of example 3 (not according to the invention), but with a polymer composition similar to example 2, shows a similarly good behavior. Also examples 4, 5, 7, 9, 9a, 10 and 11 showed a good viscosity behavior. The yield point of the composition from Example 1 was between 1 and 4 Pa. The average viscosity at a shear rate of 1 [1/s] was 23±6 [Pas]. Due to the low pH of the samples, it is possible that the gelation of the carbomer was incomplete. Example 8c showed a similar behavior. The amount of 0.42% (w/w) HA already reached a sufficient gel formation to avoid a Newtonian fluid behavior of the samples. In contrast, the samples from examples 6 and 8c showed more the behavior of a Newtonian fluid—the samples did not show a continuous gel formation.

### Example 19 Scanning Electron Microscopy (SEM) Analysis

### Sample Preparation

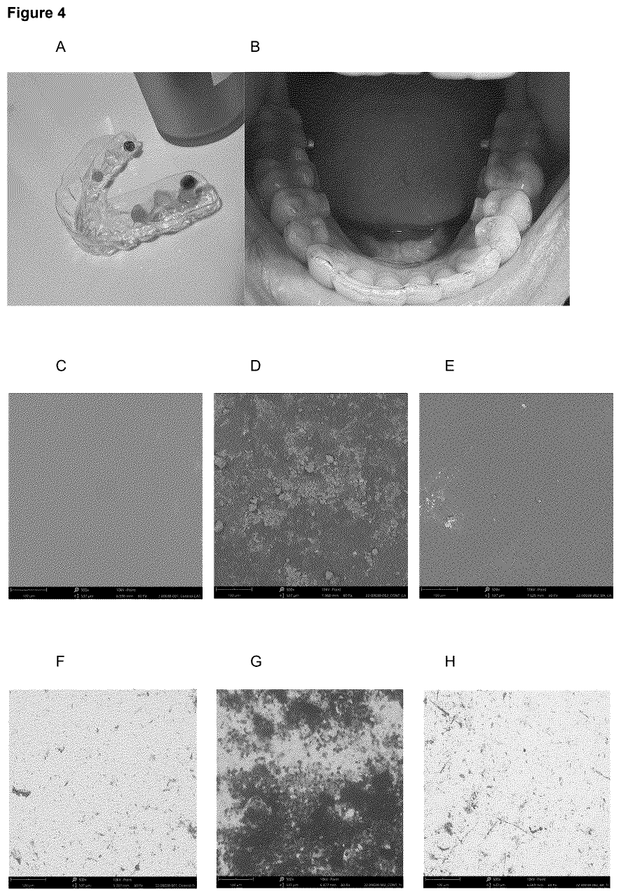

Specimens of (1) (clear aligner (CA) material (polyethylene terephthalate-glycol co-polyester) from Scheu Dental GmbH, Iserlohn, Germany), and (2) titanium (Ti) and (3) polymethylmethacrylate (PMMA) were formed as small, round plates (diameter 2.5 mm) with plane and polished surfaces. Aligner splints were prepared with two times three sections for specimens, the sections were attached to the inner aligner surface so that a specimen is cooped up between the teeth/gum and the aligner splint when the aligner splint is introduced into the oral cavity (FIGS. 4A and 4B).

Prior to the sample preparation, all specimens and aligner splints were plasma sterilized (using a Plasma Mini from Plasmapp Daegu, Seoul, Korea according to the instruction of the producer) to ensure a comparable baseline situation. The biofilm formation on the surface of the specimens (which was closest to the gum/teeth while the aligner splint was placed in the oral cavity) was determined. The first application of a sample was performed at the beginning (t=0 h) and the second application was applied after 11 h (t=11 h) of the wearing time. The aligner splints were only removed for meals (intraoral rinse has been performed after meal, no rinsing of the aligner due to possible interference with the attached specimens). The measurements were performed at 0 h and after 22 h wearing an aligner splint in triplets at random places of the surface.

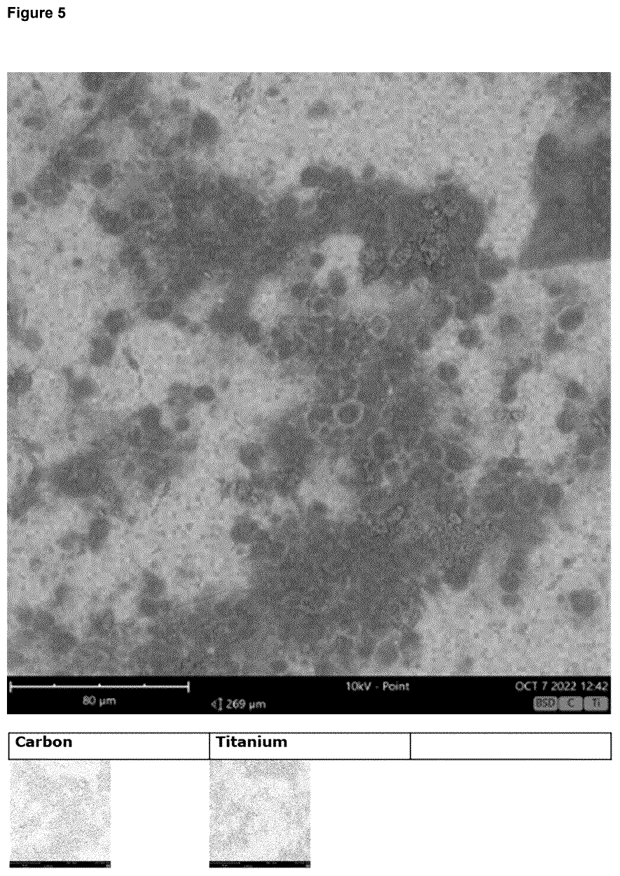

FIG. 4C to 4E show the biofilm formation on the surface of CA at 0 h (4C); at 22 h without treatment (4D); at 22 h with two treatments with Example 2 (4E). FIG. 4F to 4H shows the biofilm formation on the surface of Ti at 0 h (4F); at 22 h without treatment (4G); and at 22 h with two treatments with Example 2 (4H). Black regions on the surface at 0 h and 22 h were identified as still present deepenings on the polished titanium surface. The differentiation between biofilm formation (see (4G) and deepenings was shown by EDS mapping. FIG. 5 shows the EDS mapping of a cut out of map, here exemplarily the differentiation between Ti and biofilm of (4G) (resolution 64×64 pixels, FOV: 269 μm, Mode: 10 kV—Point, Detector: BSD Full. EDS mapping of (4F) showed no biofilm formation, EDS mapping of (4H) showed only minor biofilm formation (less than 5% which also correlates with the finding in Example 20). Although the nature of the used PMMA material and the resulting uneven surface was not optimal for these measurements, the PMMA specimen support the finding with CA and Ti.

### Example 20 Epifluorescence Microscopy

For the epifluorescence microscopy, Ti specimen were used due to the resulting black background of the specimen. Although also specimen with CA, PMMA and composite material of polyfunctional acrylates and methacrylates (10:1) which is frequently used by dentists (Merz Dental GmbH, Lütjenburg, Germany) showed the same tendency, these materials gave a green background due to their intrinsic fluorescence so the best contrast for evaluating fluorescence of stained biofilm formation was given when using Ti.

### “In Vivo”

For in vivo sample preparation, Ti specimen were prepared and incubated as described under Example 19 with the exception that only one specimen was used per side of a splint (the other two of the three sections per side were left empty). The meals as well as times of the meals during the incubation period of all samples. On each side of a splint, 0.5 ml of a respective sample was added to the inside of the splint, close to the section in which the specimen was located. In contrast to Example 19, the biofilm formation was determined after 24 h (instead after 22 h). After 24 h, the specimen were carefully removed from the splint and freezed at −80° C. until measurement.

### “In Vitro”

The same Ti specimen were prepared as described in Example 19. Saliva of 5 persons was combined. In a sealed 1.5 ml micro reaction vessel (Eppendorf, Hamburg, Germany) a Ti specimen was added with 0.5 ml combined saliva and 0.2 g of a sample. The reaction vessels were vortexed for 10 sec and then incubated for 24 h at 37° C. using a Melag 80 incubator (Melag, Medizintechnik oHG, Berlin, Germany). In contrast to Example 19, the biofilm formation was determined after 24 h (instead after 22 h). After 24 h, the specimen were carefully removed from the splint and freezed at −80° C. until measurement. For measurement, specimen were stained for 20 min in 1 ml of a 0.02% solution of acridine orange (CAS 26-94-6, Merck, Darmstadt, Germany) in sterile deionized water. The Specimens were washed twice in sterile distilled water, and air dried at room temperature. All procedures were conducted under protection from daylight. The stained specimens were examined with a Zeiss Axioplan microscope (Zeiss, Oberkochen, Germany) with a 50 W mercury high-pressure bulb, and 20×objectives (Zeiss, Plan-Neofluar) and a Zeiss filter set No. 09 (excitation: 450-490 nm; dichroic beamsplitter≥510 nm; emission≥520 nm, Zeiss, Oberkochen, Germany). The digitalized image analysis was performed using a low-light video camera and an image analyzer (both from Intas Science Imaging Instrument GmbH, Gottingen, Germany). Digitized image data were transferred to a computer and pixel size of stained microbial cells of biofilms were determined using ImageJ 1.54d (Wayne Rasband and contributors, National Institute of Health, USA) using the following settings:

In vivo - results

distance in pixels 1018,
known distance 100 μm (determined with a linear marking tool)
pixel aspect ratio =1
unit of length = μm
global
Picture type 8bit
“Strg+Shift+T” to adjust thresholds (same thresholds for similar
biofilm formations)
Analyze particles size 0-infinity, circularity 0-1

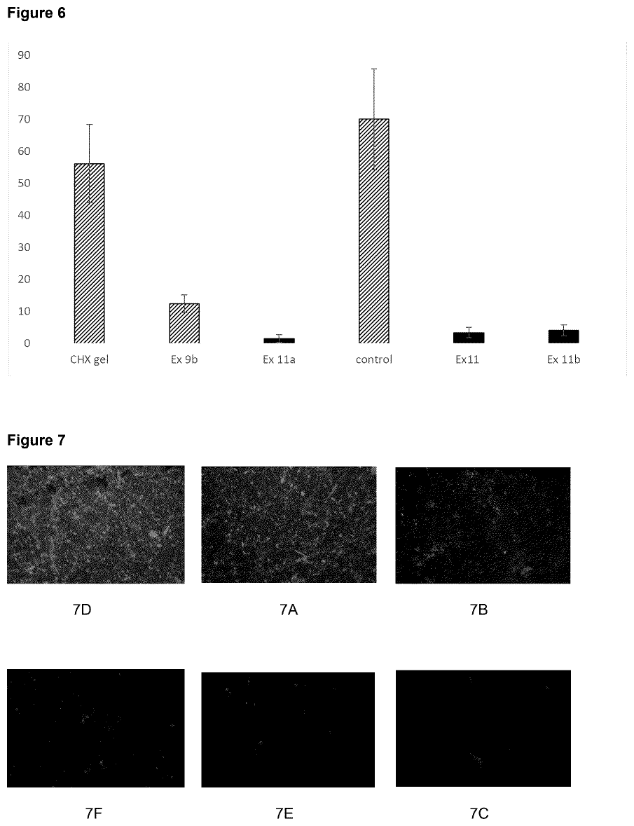

FIG. 6 shows the result of the fluorescence measurements of 10 randomly fluorescence pictures. The coverage of a Ti-surface with biofilm (y-scale of FIG. 6) was determined as described above. The control showed a wide-spread coverage of (70.1±15.83%; and an intensive fluorescence (see, e.g., FIG. 7D). The commercially available chlorhexidine gel also showed a wide-spread coverage (56.22±12.23%) but with a less intensity in fluorescence (see, e.g., FIG. 7A). Example 9b (see also FIG. 7B) which is similar to example 10 of KR1020000031162 (not according to invention) showed a high fluorescence (indicating biofilm formation) compared to Examples 11a, 11 and 11b (see FIGS. 7F, 7E, and 7C, respectively, basically no fluorescence/no biofilm formation) of which each showed a very good performance (only 1.51±1.25%; 3.36±1.58%; and 4.07±1.66% coverage, respectively). Although the amount of chlorhexidine in Example 11a is notably less (0.01% (w/w)) compared to the amount of the active ingredient in the commercially available CHX gel (0,565% (w/w)), the inhibitory effect of the sample is higher than the effect of the CHX gel. Moreover, it was surprisingly found that the effect the composition with alpha-bisabolol (Example 11) seems to have a slightly better or similarly good inhibitory effect compared with the composition with hexadecylpyridinium chloride (Example 11b). Moreover, Example 11 shows the superior effect of a formulation in accordance with the present invention compared to compositions known from the art (e.g., Example 9b). For comparison's sake, the amount of triclosan in example 10 (0.1% (w/w) in KR1020000031162) was substituted with the same amount of alpha-bisabolol in Example 9b of the present application. Although the amount of alpha-bisabolol and matricin in Example 11 are tenfold lower (0.01% (w/w)), the efficacy in inhibiting biofilm formation is notably reduced in Example 9b (coverage with biofilm 12.39±2.77% in the presence of Example 9b vs. coverage with biofilm 3.36±1.58% in the presence of Example 11 according to the invention).

Notably, the results of the in vivo and in vitro test cannot be compared with each other due to the different experimental set-ups: continuous (although reduced by the splint) salivation and the presence of gum as well as teeth surface allowing different growth conditions and allowing to bring in new bacteria or food for the latter and removing sample by (reduced) salivation on the one hand vs. constant sample and media volume in a sterile environment (reaction vessels) on the other hand.

### In Vitro—Results

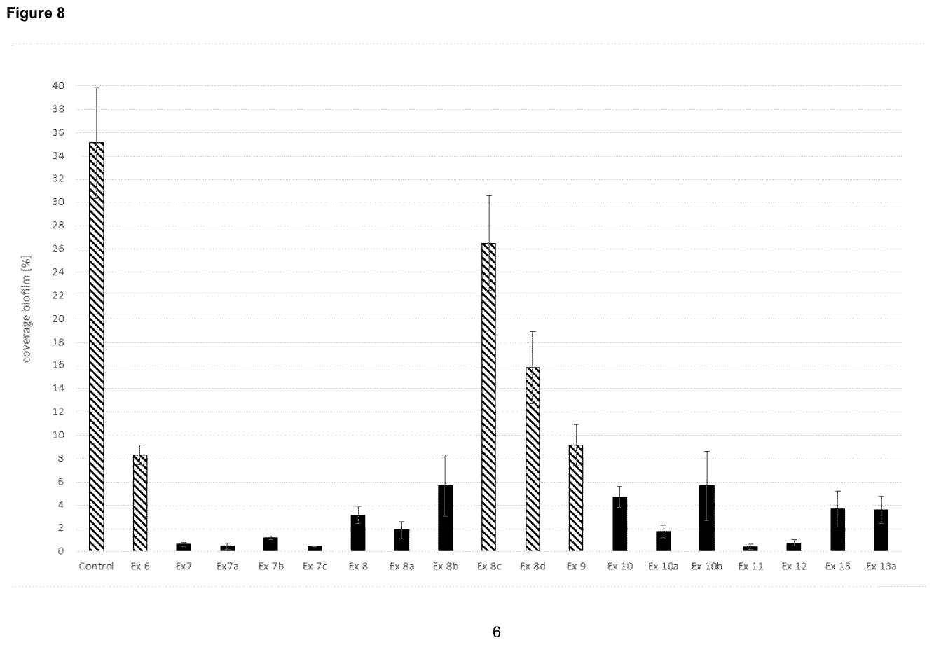

The in vitro control (water) showed after 24 h a coverage of the surface of the Ti specimen of 35.11±4.75%. Example 8c (not according to the invention) demonstrates the strongly reduced biological anti-inflammatory effect as well as the strongly reduced mucoadhesive effect due to a reduced amount of HA (0.1% (w/w)) in said formulation (resulting in a coverage of the surface with biofilm in a range of 26.51±4.09%) compared to the composition of Example 8 in accordance with the invention (3.14±0.73%), wherein the amount of HA was 1.44% (w/w) (the amount of alpha-bisabolol was identical in both formulations). In the experiment with Example 8a, wherein the amount of alpha-bisabolol was a factor 6 higher than in Example 8, the biofilm coverage of the surface was only 1.86±0.72%. Also Example 8b (amount of HA 0.42% (w/w), amount of alpha-bisabolol 0.01% (w/w) in accordance with the invention) showed much better results compared to Example 8c and good results compared to examples 8 and 8a (amount of HA 1.44% (w/w), each). Example 6 (not according to the invention, amount of HA 0.3% (w/w)) did not show as good results (biofilm coverage 8.33±0.87%) as examples 8 to 8b. In contrast, examples 7 to 7c, each comprising two water-insoluble polymers, showed very good results in regard of inhibition of biofilm formation (0.64±0.2%, 0.47±0.26%, 1.21±0.18, and 0.47±0.05). Example 9 (not according to the invention but similar to example 1 of US2020/0390676) on the other hand, showed a biofilm coverage of 9.15±1.81%, i.e. a low inhibition of biofilm formation. Actually, US2020/0390676 does not teach the use of a further inflammatory compound in combination with a specific amount of xylitol, erythritol or sorbitol but eugenol was only mentioned as a flavorant. Without being bound to the explanation, at least the low amount of xylitol (not according to the invention) results in a limited inhibitory activity of the formulation compared to the formulations of the invention. Examples 10 to 13a (in accordance with the invention, again, showed good to excellent inhibitory effects (4.69±0.88%, 1.75±0.54%, 5.67±2.97%, 0.44±0.22%, 0.76±0.27%, 3.67±1.55%, 3.61±1.14%, see also FIG. 8).

1. An oral care composition comprising:
(a) at least one protic solvent, wherein at least one protic solvent is water, and wherein the amount of the sum of all protic solvents is between 45% (w/w) and 85% (w/w), and the amount of water is at least 45% (w/w);
(b) at least one water-insoluble polymer, wherein at least one water-insoluble polymer comprises hyaluronic acid (HA) or a salt thereof, and wherein the amount of the sum of all water-insoluble polymers is between 0.9% (w/w) and 5% (w/w); and wherein the amount of hyaluronic acid is at least at least 0.8% (w/w);
(c) one or more sugar alcohol (polyol) selected from the group consisting of sorbitol, xylitol, erythritol, wherein the amount of the sum of the polyol(s) selected from the group consisting of sorbitol, xylitol, erythritol, and a combination of two or three of the said three polyols is between 15% (w/w) and 35% (w/w);
(d) at least one other anti-inflammatory component, selected from a group consisting of alpha-bisabolol, chamazulene, matricin, curcumin, beta-caryophyllene, eugenol, camphor, cineole, cuminaldehyde, thujone, chlorhexidine, povidone iodine, cetylpyridinium chloride or bromide, and ursolic acid, wherein the amount of the sum of all others anti-inflammatory components (d) is between 0.001% (w/w) and 2% (w/w);
wherein the sum of (a), (b), (c), and (d) is between 90% (w/w) and 100% (w/w).

2. The composition according to claim 1, wherein the composition comprises one or more antioxidants selected from the group consisting of L-ascorbic acid or derivatives thereof, dibutyl hydroxy toluene, butyl hydroxy anisole, superoxide dismutase, carotenoids, astaxanthin, rutin or derivatives thereof, hesperidin, quercetin, catechin; gallic acid or derivatives thereof, allantoin (N-(2,5-Dioxoimidazolidin-4-yl)urea) and derivatives thereof, glutathione or derivatives thereof, glutathione, β-carotenes or derivatives thereof, ubiquinol, polyphenols, sweet hydrangea leaf, turmeric, rose fruit, echinacea, Scutellaria root, Hypericum erectum, Chinese gall nut, Geranium thunbergii, rice, rice bran, comfrey, Xanthoxylum piperitum, labiate, peony root, soybean, “Natto” (soybeans fermented in their own bacteria), tea leaf, clove, loquat, peony, horse chestnut, saxifrage, rooibos, rosemary, spirulina, chlorella and dunaliella, bilirubin, cholesterol, tryptophan, histidine, thiotaurine and hypotaurine, wherein the amount of the sum of the said one or more antioxidant(s) is between 0.001% (w/w) and 5% (w/w), and wherein the sum of (a), (b), (c), (d) and the one or more antioxidant is between 90.0010% (w/w) and 100% (w/w).

3. The composition of claim 1, consisting of group (a), (b), (c), and (d) and one or more further component(s) (e), wherein the amount of the sum of all further components (e) is 10% (w/w) or less, optionally, wherein the further components (e) are selected from the group consisting of (e1) (extracts and oils), (e2) buffers, (e3) chelating agents, (e4) antioxidants, (e5) preservatives, (e6) humectants, (e7) flavorings, (e8) vitamins, (e9) retinoids, (e11) amino acid, (e12) oral cavity fortifying polyols, (e13) hydroxy acids, (e14) fluoride, (e15) sweeteners, (e16) colorants, (e17) peptides, (e18) cooling agents, (e19) warming agents, (e20) tingling agents, (e21) emulsifier, (e22) further additives; and wherein the sum of (a), (b), (c), (d), and (e) is 100%.

4. (canceled)

5. The composition according to claim 1, wherein (b) comprises HA and at least one further water-insoluble polymer selected from the group consisting of a carbomer, a mannan, and a cellulose.

6. The composition according to claim 1, wherein the HA or sodium salt thereof has a molar mass between 5 kDa and 2000 kDa.

7. The composition according to claim 1, wherein (c) consists of one or more polyol selected from the group consisting of xylitol and erythritol.

8. The composition according to claim 1, wherein (d) consists of one or more compound(s) selected from the group consisting of alpha-bisabolol, chamazulene, matricin, eugenol, curcumin, beta-caryophyllene, chlorhexidine, povidone iodine, cetylpyridinium chloride, and cetylpyridinium bromide.

9. The composition according to claim 1, wherein the viscosity of the composition at a shear rate of 1 [1/s] and a temperature of 22° C., is between 10 Pas and 800 Pas.

10. The composition according to claim 1, wherein the pH of the total composition is between pH 6 and pH 8, optionally, between 6 0.8 and 7 0.8.

11. The composition according to claim 1, wherein the composition does not comprise triclosan (5-chloro-2-(2,4-dichlorophenoxy)phenol).

12. The composition according to claim 1, wherein the amount of each component selected from the group consisting of calcium pyrophosphate, calcium hydroxyapatite, diatomaceous earth, wollastonite, perlite, PMMA, silicon beads is, if present, is 0.00001% (w/w) or less; the amount of dicalcium phosphate, if present, is 0.008% (w/w) or less; the amount of each component selected from the group consisting of tricalcium phosphate, calcium metaphosphate, and calcium polyphosphate is 0.0016% (w/w) or less; the amount of calcium carbonate is 0.001% (w/w) or less; the amount of strontium carbonate in a composition is 0.0008% (w/w) or less; the amount of sodium bicarbonate is 7.4% (w/w) or less, optionally 1% (w/w) or less; the amount of fused silica is 0.0008% (w/w) or less; the amount of each component selected from the group consisting of fumed silica, precipitated silica, hydrated silica, and silicon beads is 0.0009% (w/w) or less; the amount of barium sulfate is 0.0017% (w/w) or less.

13. The composition according to claim 1, that does not comprise calcium pyrophosphate, calcium hydroxyapatite, diatomaceous earth, wollastonite, perlite, PMMA, silicon beads dicalcium phosphate tricalcium phosphate, calcium metaphosphate, calcium polyphosphate, calcium carbonate strontium carbonate, fused silica, fumed silica, precipitated silica, hydrated silica, silicon beads, and/or barium sulfate.

14. (canceled)

15. (canceled)

16. The oral care composition according to claim 1, wherein (d) is selected from the group consisting of alpha-bisabolol, chamazulene, matricin, eugenol, curcumin, beta-caryophyllene, camphor, cineole and thujone, and ursolic acid.

17. The oral care composition according to claim 1, wherein (d) is selected from the group consisting of alpha-bisabolol, chamazulene, matricin, or a combination of two or three thereof.

18. The composition according to claim 1, wherein the composition comprises one or more catechins wherein the amount of the sum of the said one or more catechins is between 0.0010% (w/w) and 5% (w/w), and wherein the sum of (a), (b), (c), (d) and the one or more catechins between 90.001% (w/w) and 100% (w/w).

19. A method to improve gum health in the oral cavity of a subject comprising using a composition of claim 1.

20. The method according to claim 19, wherein the subject is wearing an aligner splint or a mouth guard.

21. A method to care for gum when wearing a splint comprising applying a composition according to claim 1 to teeth and/or gum and/or on said splint.

22. A method for preparing a composition according to claim 1, comprising dispersing HA in ethanol or in an ethylene glycol comprising medium and adding the aqueous phase, wherein further components can be added either to the aqueous phase prior to mixing with a HA dispersion and/or can be added after the HA and aqueous phase were combined, under stirring; or adding HA to an ethanol or ethylene glycol comprising product before adding the aqueous phase, wherein further components can be added either to the aqueous phase prior to mixing with a HA dispersion and/or can be added after suspended HA and the aqueous phase were combined, under stirring; and/or one or more components are added to the HA dispersion and others to the aqueous phase prior to mixing with HA and/or can be added after the HA and the aqueous phase are combined.
